SEQUENCE LISTING (11 sequences)

SEQ ID NO: 1 - protein, 5 aa, synthetic construct
GGGGS

SEQ ID NO: 2 - protein, 9 aa, synthetic construct
GGGGGGGGG

SEQ ID NO: 3 - protein, 9 aa, synthetic construct
EEEEEEEEE

SEQ ID NO: 4 - protein, 9 aa, synthetic construct
SSSSSSSSS

SEQ ID NO: 5 - protein, 9 aa, synthetic construct
GGGGGCPPC

SEQ ID NO: 6 - protein, 15 aa, synthetic construct
GGGGSGGGGSGGGGS

SEQ ID NO: 7 - protein, 13 aa, synthetic construct
SCVPLMRCGGCCN

SEQ ID NO: 8 - protein, 14 aa, synthetic construct
PSCVPLMRCGGCCN

SEQ ID NO: 9 - protein, 9 aa, synthetic construct
GDLIWRNQK

SEQ ID NO: 10 - protein, 23 aa, synthetic construct
GGGGGGGGGPSCVPLMRCGGCCN

SEQ ID NO: 11 - protein, 4 aa, synthetic construct
GTGA

(19) United States
(12) Patent Application Publication
(10) Pub. No.: US 2026/0049331 A1
(43) Pub. Date: Feb. 19, 2026

(54) Sense-and-Response of Proteins, Peptides, and Small Molecules Using Ligand-Induced Dimerization Activating RNA Editing (LIDAR)

(71) Applicant: The Board of Trustees of the Leland Stanford Junior University, Stanford, CA (US)

(72) Inventors: Xiaojing Gao, Stanford, CA (US); Xiaowei Zhang, Stanford, CA (US); Kristjan E. Kaseniit, Stanford, CA (US); Luis Santiago Mille Fragoso, Stanford, CA (US); Connor C. Call, Stanford, CA (US)

(21) Appl. No.: 19/100,486

(22) PCT Filed: Aug. 16, 2023
(86) PCT No.: PCT/US2023/072322

§ 371 Date: Jan. 31, 2025

Related U.S. Application Data
(60) Provisional application No. 63/399,400, filed on Aug. 19, 2022.

(51) Int. Cl. C12N 15/85; C07K 14/00; C12N 9/78; C12N 15/11

(52) U.S. Cl. CPC C12N 15/85; C07K 14/00; C12N 9/78; C12N 15/11; C12Y 305/04004; C07K 2319/85; C12N 2840/002

(57) ABSTRACT

The present disclosure provides a method for ligand induced ADAR (adenosine deaminase acting on RNA) mediated expression of an RNA coding sequence, the method comprising contacting a ligand with a cell associated LIDAR meditated expression system comprising: 1) an output RNA, 2) a stem loop binding protein and 3) an RNA editing protein; to express the RNA coding sequence. The present disclosure also provides compositions and kits for practicing the methods disclosed herein.

### CROSS-REFERENCE TO RELATED APPLICATIONS

Pursuant to 35 U.S.C. § 119 (c), this application claims priority to the filing date of U.S. Provisional Patent Application Ser. No. 63/399,400 filed Aug. 19, 2022, the disclosure of which application is herein incorporated by reference.

### GOVERNMENT RIGHTS

This invention was made with Government support under contract EB027723 awarded by the National Institutes of Health. The Government has certain rights in the invention.

### REFERENCE TO AN ELECTRONIC SEQUENCE LISTING

The contents of the electronic sequence listing (STAN-2009WO_Seq_List.xml; Size: 10,688 bytes; and Date of Creation: Aug. 16, 2023) is herein incorporated by reference in its entirety.

### INTRODUCTION

Recent developments of modular synthetic receptors have enabled cells to detect diverse extracellular ligands and generate customized outputs (1). They have empowered basic research and biomedical applications, such as visualizing neurotransmitter release (2) and increasing the targeting specificity of chimeric antigen receptor (CAR) T cells (3). Meanwhile, the success of mRNA vaccines demonstrates a transformative modality of therapeutic delivery, because its transient and non-mutagenic nature offers superior safety over traditional DNA vectors (4). For example, mRNA-mediated CAR expression in vivo holds the promise to substantially decrease the cost of such therapies. Given that all existing modular synthetic receptors operate through transcriptional regulation, there remains a critical opportunity at the interface of these two thriving fields-RNA-compatible synthetic receptors based on novel post-transcriptional actuation mechanisms.

Provided herein are modular post-transcriptional synthetic receptors and methods for using said synthetic receptors for the production of desired output polypeptides.

### SUMMARY

The present disclosure provides a method for ligand induced ADAR (adenosine deaminase acting on RNA) mediated expression of an RNA coding sequence, the method comprising contacting a ligand with a cell associated LIDAR meditated expression system comprising: 1) an output RNA, 2) a stem loop binding protein and 3) an RNA editing protein; to express the RNA coding sequence.

Methods for treating a disease or condition are provided.

Compositions and kits for practicing the subject methods are also provided.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Three different classes of LIDAR sensors. (A-C) Schematics of cLIDAR, eLIDAR and tLIDAR systems. (D-F) Preliminary data: histograms of output EGFP fluorescence for each type of LIDAR sensors: (D) rapalog-sensing cLIDAR, (E) rapalog-sensing eLIDAR, (F) Arginine vasopressin (AVP)-sensing tLIDAR. Black dots indicate mean of each replicate, green lines indicate mean of each replicate's mean. (G-K) exemplary configurations of LIDAR mediated expression systems, (G) tLIDAR where the dimerization domain of the stem loop binding protein is a GPCR and the dimerization domain of the RNA editing protein is a protein that binds to the dimerization domain of the stem loop binding protein following ligand binding, (H) tLIDAR where the dimerization domain of the RNA editing protein is a GPCR and the dimerization domain of the stem loop protein is an antibody fragment that binds to the dimerization domain of the RNA editing protein following binding to the ligand. (I) tLIDAR where the dimerization domain of the stem loop binding protein is a GPCR and the dimerization domain of the RNA editing protein is an antibody fragment that binds to the dimerization domain of the stem loop binding protein following binding to the ligand, (J) tLIDAR where the dimerization domain of the RNA editing protein is a receptor tyrosine kinase (RTK; i.e. insulin growth factor 1 receptor) and the dimerization domain of the stem loop protein is a protein (i.e. Shc) that binds to the dimerization domain of the RNA editing protein following binding to the ligand and tLIDAR where the dimerization domain of the stem loop binding protein is a RTK (i.e. insulin growth factor 1 receptor) and the dimerization domain of the RNA editing protein is a protein (i.e. Shc) that binds to the dimerization domain of the stem loop binding protein following binding to the ligand, (K) tLIDAR where the dimerization domain of the RNA editing protein is a light sensitive protein (i.e. rhodopsin) and the dimerization domain of the stem loop protein is a protein (i.e. visual arrestin) that binds to the dimerization domain of the RNA editing protein following light stimulation and tLIDAR where the dimerization domain of the stem loop binding protein is a light sensitive protein (i.e. rhodopsin) and the dimerization domain of the RNA editing protein is a protein (i.e. visual arrestin) that binds to the dimerization domain of the stem loop binding protein following light stimulation.

FIG. 2. Two different eLIDAR receptors. (A) Receptor in FIG. 1A responds to externally administered AVP. Biological triplicates fit with a Hill equation. (B, C) Histograms of eLIDAR receptors' responses to rapalog and IL-2 delivered at saturating levels. IL-2 is expressed from a plasmid co-transfected with LIDAR-encoding plasmids. Green lines indicate mean.

FIG. 3. eLIDAR baseline reduction. (A) Origin of baseline in the secretory pathway. (B, C) Two strategies to inhibit baseline in the secretory pathway using catalytically inactive ADAR2dd.

FIG. 4. Secreted outputs. (A) Direct secretion. (B) Mediated by proteolytic removal of ER retention motif.

FIG. 5 Alternative LIDAR designs. (A) tLIDAR based on recruitment of She by activated IL-2 receptor. (B) Repurposed protease-activated GPCR responsive to ligand-reconstituted HCV protease. The agonist peptide is sequestered till cleavage.

FIG. 6 Tuning LIDAR. Compared to the basic motif (middle), positive feedback increases ultrasensitivity and competitive inhibition raises threshold.

FIG. 7 depicts an exemplary oligonucleotide sequence encoding the output RNA, the stem loop binding protein and the RNA editing protein.

FIG. 8 Design of a modular post-transcriptional synthetic receptor architecture. (A) The general LIDAR (Ligand-Induced Dimerization Activating RNA editing) architecture consists of three main components. The first component incorporates a dimerization module fused to an RNA Binding Protein (RBP, i.e., MCP). The second component contains the other dimerization module fused to an ADAR2dd which can edit dsRNAs when in close proximity. The third component is a reporter RNA harboring an in-frame stop codon within a double-stranded stem loop that functions as an ADAR substrate, and RNA stem loops that recruit specific RBPs (i.e., MS2 that recruits MCP). The stem-loop is placed in between the marker and output flanking with F2A self-cleavage peptide, preventing translation of the output. The presence of ligand brings the two dimerization modules together, such that ADAR2dd comes in proximity to the reporter RNA, triggering editing of the UAG stop codon into a UIG tryptophan codon, leading to payload translation. (B) The LIDAR architecture is used to implement a cytosolic ligand sensor. (C) an extracellular ligand receptor. (D) a GPCR-based receptor, and (E) a RTK-based receptor. (F-I) Data for each of the implementations. Fluorescent intensities and activation fold-change for induces versus uninduced for (F) cytosolic LIDAR, (G) extracellular LIDAR, (H) GPCR LIDAR and (1) RTK LIDAR.

FIG. 9 Modularity of inputs and outputs for the LIDAR architecture. (A) Diagram showing the implementation of an IL-2 receptor using extracellular LIDAR. (B) Data showing the activation of the extracellular LIDAR and the production of the output triggered by the presence of IL-2. (C) Schematic of the general structure for GPCR LIDAR. (D) Different GPCRs are compatible with the LIDAR architecture showing high activation levels. (E) Diagram showing the capacity of LIDAR to drive cell apoptosis by producing a functional iCasp9. (F) Quantification of the remaining live cells after LIDAR-induced cell apoptosis. Additional data and information on this assay can be found in FIG. S3 and the Methods section. (G) Schematic demonstrating the versatility of LIDAR and its capacity to interface with existing molecular systems and circuits such as RELEASE (17) and protease-based circuitry (14). (H) Data showing fine control of protein stability by a LIDAR-produced protease. (I) LIDAR can efficiently interface with RELEASE and produce a significant response. All cells are transfected with a membrane-bound human IgG retained in the ER by a C-terminal-RXR motif and a TEVP cut site in the middle. Full LIDAR-RELEASE: co-transfection with a full CCR6-based LIDAR system; TEVP receptor: co-transfection with a LIDAR reporter with TEVP output; TEVP positive control: co-transfection with a LIDAR positive control reporter (UAG stop codon replaced with UGG codon).

FIG. 10 LIDAR can drive cell-cell interactions and complex cellular behaviors. (A) LIDAR can detect ligands secreted by other cells, which shows (B) significant difference in activation levels between uninduced and induced conditions. Supernatant from sender cells was transferred to the receiver cells after 24 h of culture (see Method). Flow cytometry was run 24 h after addition of supernatant. (C) LIDAR is also capable of detecting ligands bound to the membrane of other cells. (D) Sender and receiver cells were co-cultured together and flow cytometry was run 24 h after seeding. Data is from mCherry positive (receiver) cells, gating out BFP positive (receiver) cells. (E) Spatial patterns were created using generated stable cell lines (cLSM01-C5). Sender cells producing secreted CCL20 (cLSM03) were co-cultured with receiver cells (cLSM01-C5) in a circular pattern to demonstrate LIDARs sensing capabilities and sensitivity and its potential to produce complex spatial patterns. Diagram (left) shows the experimental setup and pictures (right) portrait the seeded sender (blue) and receiver (red) cells. An overlay of all channels showing sender, receiver and activated receiver cells in a single image (far right) is also portrait. (F) GFP channel showing activated receiver cells. For soluble ligands we see a gradient from the center of the circular pattern outwards. (G) Sender cells producing membrane-bound CCL20 (XZcp07) were co-cultured with receiver cells (cLSM01-c5) in a similar fashion as with sender cells producing secreted ligands. Individual panels are the same as for the secreted ligand condition. (H) GFP channel showing activated receiver cells. For membrane-bound ligands we see a single and more uniform line as receiver cells only get activated in the direct interface between sender and receiver cells. Overall GFP intensity is lower compared to the soluble ligand condition due to weaker activation as demonstrated in FIG. 3A. D. (I) Concentric intensity profiles for both secreted and membrane-bound conditions. Average intensity values are used. A graphical demonstration of how these average values are calculated can be seen in Supplementary FIG. 3. Triplicates were done for each condition. All pictures were taken using an EVOS M700 imaging system. Refer to the Methods section for more information on the seeding and co-culture process, image acquisition and processing and quantification of the intensity profiles. (J) Schematic demonstrating how multiple different GPCR-LIDARs can be implemented in a single cell (oLIDAR) to orthogonally sense different inputs and produce separate outputs. (K) Data showing the implementation of both AVPR-LIDAR and CX3CR1-LIDAR in the same cell. The y-axes for BFP and GFP are different to portrait both baselines at similar apparent levels to account for intrinsic differences of the intensities of these two fluorophores.

FIG. 11 LIDAR is RNA-compatible and can be encoded as a single transcript. (A) Schematic showing single transcript encoding of LIDAR. (B) Data showing the successful transient transfection and functioning of a single transcript LIDAR based on AVPR2. (C) Experimental setup of an RNA-delivered CCR6-LIDAR. Each LIDAR component was delivered as individual mRNA. RNA transfection was done with 500 ng of each LIDAR component, mRNA of LIDAR reporter RNA is synthesized without modified bases, while mRNA of CCR6-ADAR2dd and ARRB2-MCP are synthesized with 100% N1-methyl-pseudouridine. (D-E) Efficient signal activation with RNA-based LIDAR system gating on marker highly expressed cells.

FIG. 12 Optimization of the LIDAR components. (A) Schematic of the LIDAR reporter RNA and its predicted folding structure. A GluR-B derived stem loop is used as the ADAR substrate (6) and 3× MS2 loops are included in the design to ensure strong MCP recruitment. (B) Data showing the differences between having 1, 2 or 3 RNA binding protein substrates (i.e., MS2) loops in the output levels for cytosolic LIDAR and (C) extracellular LIDAR. (D-E) Supporting data for the rationale behind using a double-mutant ADAR2dd for LIDAR. The double mutant ADAR2dd was tested in (D) cytosolic LIDAR and (E) GPCR LIDAR based on AVPR2. The results for panels (B-E) are representative and can be extrapolated to other LIDAR designs. (F) Data showing the performance of an inverted GPCR LIDAR design. Depending on the GPCR, fold activation can reach 17×, which is lower compared to the chosen design. (G) GPCR LIDAR receptors perform differently depending on the composition of their C-terminal tails. GPCR LIDAR receptors with low background benefit from an extra V2 tail at their C-terminus.

FIG. 13 Robustness and limitations of the LIDAR system. (A) Direct comparison between a TF-based synthetic receptor and LIDAR based on the same GPCR AVPR2. (B) Dose responsiveness and (C) time responsiveness curves for an AVPR2-based GPCR-LIDAR. (D) Close up to the first 4 h after AVPR LIDAR induction depicts a 2.5-fold increase after 1 h of ligand induction. (E) Dose responsiveness curve for CCR6 LIDAR stably integrated and (F) transiently transfected. (G) LIDAR is not efficient in directly output payloads that go through the secretory pathway. Left: schematics of A GPCR LIDAR directly output a membrane-bound protein. Right: benchmarking results using a membrane bound human IgG1 as direct LIDAR output (with Igk leader signal peptide). Cells are stained with anti hIgG1 AF488 to quantify membrane expression, and fluorescent intensities for each individual cell are normalized with marker mCherry intensities. No significant increase in membrane expression of IgG1 was observed when transfected with a full LIDAR system.

FIG. 14 Co-culture for pattern formation and induced apoptosis experiments. (A) Fabrication of silicone inserts and seeding of sender and receiver cells for pattern formation experiments. A detailed explanation of the steps can be found in the Methods section. Cells are seeded in separate compartments using a PDMS insert with walls of approximately 500 μm. The day after seeding, the PDMS insert is removed to allow cells to grow and to come into contact with each other. After a couple of days, a pattern can be seen. (B) Additional patterns generated by the pattern formation experiment. Two more patterns are shown for both conditions, secreted and membrane-bound ligands. (C) Graphic representation of how the average intensity values were obtained to generate the intensity profiles for the patterns generated in the co-culture experiments. Each concentric red line represents a data point in the plots shown in FIG. 3. For each figure. 150 points-concentric red lines-were taken. Each of these points is the average value of 10,000 intensities. (D) Pictures of each of the conditions for the LIDAR induced cell apoptosis experiments. Death cells (top) can be seen only for the condition where ligand (CCL20) and iCasp9 dimerization molecule (AP1903) was administered to the cells (13). Live cells are seen for the rest of the conditions. Brightfield images with death cells marked in red (left), as well as original individual channel images are shown. Cells in green are expressing iCasp9.

FIG. 15 LIDAR is compatible with mRNA encoding and delivery. (A) mCherry MFI of cells transfected with LIDAR reporter mRNA without modified bases or with 25% N1-methyl-pseudouridine. RNA stability is enhanced by introducing N1-methyl-pseudouridine as seen by an increase and accumulation of the marker mCherry. (B) EGFP fluorescent intensity of cells transfected with LIDAR sensor gating on highly transfected population based on mCherry. Leaky baseline was observed with reporter RNAs containing modified bases, leading to lower fold activation.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It must be noted that as used herein and in the appended claims, the singular forms “a”, “an”, and “the” include plural referents unless the context clearly dictates otherwise. For example, the term “an output RNA” refers to one or more output RNAs, i.e., a single output RNA and multiple output RNAs. It is further noted that the claims can be drafted to exclude any optional element. As such, this statement is intended to serve as antecedent basis for use of such exclusive terminology as “solely.” “only” and the like in connection with the recitation of claim elements, or use of a “negative” limitation.

The terms “polynucleotide” and “nucleic acid.” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer containing purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

By “hybridizable” or “complementary” or “substantially complementary” it is meant that a nucleic acid (e.g. RNA, DNA) has a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). Inosine (I) bases pair with cytosine/cytidine. In addition, for hybridization between two RNA molecules (e.g., dsRNA), and for hybridization of a DNA molecule with an RNA molecule (e.g., when a DNA target nucleic acid base pairs with a sensor RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a sensor RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a sensor RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a protein, a cell, or an organism, refers to a nucleic acid, protein, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “exogenous” as used herein as applied to a nucleic acid or a protein refers to a nucleic acid or protein that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell. As used herein, the term “endogenous polypeptide” refers to a polypeptide that is normally found in and/or produced by a given bacterium, organism, or cell in nature.

“Recombinant,” as used herein, means that a particular nucleic acid or protein is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA containing the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms.

Thus, e.g., the term “recombinant” nucleic acid or “recombinant” protein refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

By “construct” or “vector” is meant a recombinant nucleic acid, generally recombinant DNA, which has been generated for the purpose of the expression and/or propagation of a nucleotide sequence(s) of interest, or is to be used in the construction of other recombinant nucleotide sequences.

The term “transformation” or “transfection” refers to a permanent or transient genetic change induced in a cell following introduction of a nucleic acid (i.e., DNA and/or RNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel et al, Short Protocols in Molecular Biology, 3rd ed., Wiley & Sons, 1995.

The terms “regulatory region” and “regulatory elements”, used interchangeably herein, refer to transcriptional and translational control sequences, such as promoters, enhancers, polyadenylation signals, terminators, start and stop codons, upstream open reading frames (uORFs), protein degradation signals, and the like, that provide for and/or regulate expression of a coding sequence and/or production of an encoded polypeptide in a host cell. As used herein, a “promoter sequence” or “promoter” is a DNA regulatory region capable of binding/recruiting RNA polymerase (e.g., via a transcription initiation complex) and initiating transcription of a downstream (3′ direction) sequence (e.g., a protein coding (“coding”) or non protein-coding (“non-coding”) sequence. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein), it may be a spatially restricted promoter (e.g., tissue specific promoter, cell type specific promoter, etc.), and/or it may be a temporally restricted promoter (e.g., the promoter is in the “ON” state or “OFF” state during specific stages of embryonic development or during specific stages of a biological process, e.g., hair follicle cycle in mice).

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non protein coding sequence, e.g., a sequence encoding a Shh protein; and the like) if the promoter affects its transcription and/or expression.

The term “adenosine deaminase acting on RNA” or “ADAR” refers to an enzyme that catalyze the hydrolytic C6 deamination of adenosine (A) to produce inosine (I) in RNA substrates that are double stranded. ADARs preferentially edit double stranded RNAs at sites of mismatches where mismatches containing adenosines and cytosines are editing more efficiently than other mismatches. Editing by ADARs results in nucleotide substitution in RNA, because the purine I generated as the result of the deamination reaction is recognized as G instead of A, both by ribosomes during translational decoding of mRNA and by RNA-dependent polymerases during RNA replication. The term “ADAR” encompasses any known type of ADAR such as ADAR1 (ADAR) or ADAR2 (ADARB2).

As used herein “ADAR1” refers to an adenosine deaminase acting on RNA that catalyze the hydrolytic C6 deamination of adenosine (A) to produce inosine (I) in RNA substrates that are double stranded. ADAR1 has 2 main isoforms, p150 and p110. The term “ADAR1” encompasses ADAR1 from various species. Amino acid sequences of ADAR1 from various species are publicly available. See, e.g., GenBank Accession Nos. NP_001102 (Homo sapiens ADAR1 p150), NP_001180424.1 (Homo sapiens ADAR1 p110), NP_001139768 (Mus musculus ADAR1 p150). NP_001033676 (Mus musculus ADAR1 p110). The term “ADAR1” as used herein also encompasses fragments, fusion proteins, and variants (e.g., variants having one or more amino acid substitutions, addition, deletions, and/or insertions) that retain ADAR1 enzymatic activity.

As used herein “ADAR2” refers to an adenosine deaminase acting on RNA that catalyze the hydrolytic C6 deamination of adenosine (A) to produce inosine (I) in RNA substrates that are double stranded. ADAR2 is exclusively localized to the nucleus. The term “ADAR2” encompasses ADAR2 from various species. Amino acid sequences of ADAR2 from various species are publicly available. See, e.g., GenBank Accession Nos. NP_056648.1 (Homo sapiens ADAR2), NP_001020008.1 (Mus musculus ADAR2), ACO52474.1 (Doryteuthis opalescens ADAR2). The term “ADAR2” as used herein also encompasses fragments, fusion proteins, and variants (e.g., variants having one or more amino acid substitutions, addition, deletions, and/or insertions) that retain ADAR2 enzymatic activity.

The term “modified ADAR” typically, although not necessarily, refers to a truncated ADAR, an ADAR containing amino acid substitutions or a combination thereof. Modified ADAR polypeptides often primarily have the deaminase domain of an ADAR1 or ADAR2 polypeptide. Of particular interest are deaminase domains having amino acid substitutions or truncations that enhance the deaminase/editing activity of the deaminase domain. Modified ADARs with enhanced deaminase/editing activity are known in the art, for example, as disclosed by Katrekar et al. (Elife. 2022 Jan. 19; 11:e75555), Matthews et al. (Nat Struct Mol Biol. 2016 May; 23(5):426-33), Katrekar et al. (Nat Methods. 2019 March; 16(3):239-242).

As used herein “ADAR responsive site” refers to any portion of a nucleotide sequence that is capable of being edited by an ADAR polypeptide. ADAR polypeptides edit double strand segments of RNA nucleotide sequences. The double stranded RNA nucleotide sequences may be in the form of a linear or circular double stranded RNA nucleotide sequence or may be in the form of a hairpin or hairpin-like structure in the double stranded RNA nucleotide sequence.

The term “cell associated” as used herein refers to a nucleotide or polypeptide sequence that is within close proximity, bound, embed within, or contained within a cell. The nucleotide or polypeptide sequence may be extracellular and in close proximity to the surface of the cell. The nucleotide or polypeptide sequence may be bound to a protein, lipid, or small molecule present on the cell surface. The nucleotide or polypeptide sequence may be embedded within the plasma membrane, outer membrane, capsule or cell wall of the cell, e.g., a polypeptide containing a transmembrane domain. The nucleotide or polypeptide sequence may be intracellular and contained within the cell, e.g., in the cytoplasm or within a specific cell compartment.

The term “dimerization domain” as used herein refers to a polypeptide sequence or a segment thereof that facilitates the binding of two distinct polypeptides, e.g., a first and second dimerization domain. Dimerization domains may directly or indirectly interact through a dimerization mediator, e.g., a ligand. A dimerization domain may also be a polypeptide or segment thereof that becomes post-translationally modified following the binding of a ligand which then allows binding to another polypeptide having a dimerization domain. In some embodiments, the dimerization domain is a G-coupled protein receptor (GPCR) or a fragment thereof. In some embodiments, the dimerization domain is a receptor tyrosine kinase (RTK) or a fragment thereof.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The term “biological sample” encompasses a clinical sample or a non-clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's sample cell, e.g., a sample containing polynucleotides and/or polypeptides that is obtained from a patient's sample cell (e.g., a cell lysate or other cell extract containing polynucleotides and/or polypeptides); and a sample containing sample cells from a patient. A biological sample containing a sample cell from a patient can also include normal, non-diseased cells. A biological sample may be from a plant or an animal. The biological sample may also be from any species. In certain embodiments of the method, the biological sample includes a cell. In some instances of the method, the cell is in vitro. In some instances of the method, the cell is in vivo.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fᵥ molecules (sFv) (see, e.g., Huston et al. (1988) Proc Natl Acad Sci USA 85:5879-5883); nanobodies (see, e.g., Hamers-Casterman et al. (1993) Nature 363:446; Desmyter et al. (2015) Curr. Opin. Struct. Biol. 32:1); dimeric and trimeric antibody fragment constructs; minibodies (see. e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

A “single-chain antibody,” “single chain variable fragment,” or “scFv” has an antibody heavy chain variable domain (VH) and a light-chain variable domain (VL) joined together by a flexible peptide linker. The peptide linker is typically 10-25 amino acids in length. Single-chain antibodies retain the antigen-binding properties of natural full-length antibodies, but are smaller than natural intact antibodies or Fab fragments because of the lack of an Fc domain.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” include old world camelids (Camelus bactrianus and Camelus dromedarius) and new world camelids (for example, Llama paccos. Llama glama, Llama guanicoe and Llama vicugna). A single variable domain heavy chain antibody is referred to herein as a nanobody or a V_{HH} antibody. Nanobodies are smaller than human antibodies, where nanobodies are generally 12-15 kDa, human antibodies are generally 150-160 kDa. Fab fragments are ˜50 kDa and single-chain variable fragments are ˜25 kDa. Nanobodies provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e., cheaper production costs relative to animal immunization), and the like. Specific nanobodies have been successfully generated using yeast surface display as shown in McMahon et al. (2018) Nature Structural Molecular Biology 25 (3): 289-296 which is specifically incorporated herein by reference.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

In further describing the subject invention, methods for ligand induced ADAR mediated expression of an RNA coding sequence described first in greater detail. Next, compositions for practice the methods disclosed herein. Kits are also described.

Methods for Ligand Induced Adar (Adenosine Deaminase Acting on RNA) Mediated Expression of an RNA Coding Sequence

The present disclosure provides a method for ligand induced ADAR (adenosine deaminase acting on RNA) mediated expression of an RNA coding sequence, the method including contacting a ligand with a cell associated ligand induced ADAR (LIDAR) meditated expression system including: 1) an output RNA. 2) a stem loop binding protein and 3) an RNA editing protein; to express the RNA coding sequence.

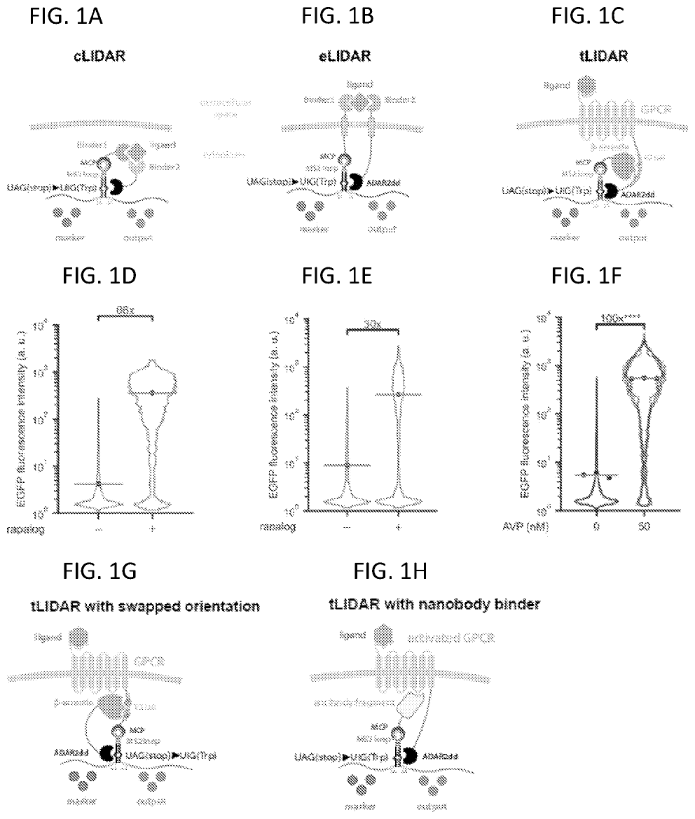

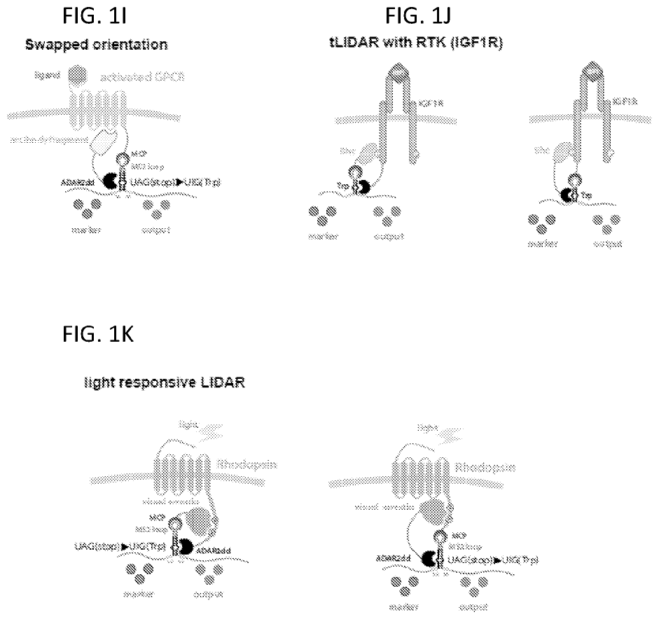

The ligands of the present disclosure may be any ligand that facilitates the dimerization of the dimerization domain of the stem loop binding protein and the dimerization domain of the RNA editing protein. The ligand may induce direct dimerization, i.e., both the dimerization domain of the stem loop binding protein and the RNA editing protein bind directly to one another such as disclosed in FIG. 1H, or indirectly, i.e., both the dimerization domain of the stem loop binding protein and the RNA editing protein bind to the ligand such as disclosed in FIG. 1B. In some embodiments, the ligand is an exogenous ligand to the cell of the cell associated LIDAR mediated expression system. In some embodiments, the ligand is an endogenous ligand to the cell of the cell associated LIDAR mediated expression system. In some embodiments, the ligand is any ligand disclosed in Table 1 or Table 2. In some embodiments, the ligand is a synthetic ligand. In some embodiments, the synthetic ligand only binds to the dimerization domain of the stem loop binding protein and/or the dimerization domain of the RNA editing protein.

In some embodiments, the ligand is light. In embodiments where the ligand is light, the dimerization domain of the stem loop binding protein or the dimerization domain of the RNA editing protein is a light sensitive protein. In embodiments where the dimerization domain is a light sensitive protein, the light sensitive protein may be any protein that light sensitive protein that is sensitive to a specific wavelength of light. Non-limiting examples of light sensitive proteins include, without limitation, eNpHr, eNpHr2.0, eNpHr3.0, eNpHr3.1, GtR3, “C1V1”, channel rhodopsin 1 (ChR1), VChR1, channel rhodopsin 2 (ChR2) etc. Additional information regarding other light-activated cation channels, anion pumps, and proton pumps can be found in U.S. Patent Application Publication Nos: 2009/0093403; and International Patent Application No: PCT/US2011/028893, each specifically incorporated by reference herein. When the light sensitive protein is eNpHr, eNpHr2.0, eNpHr3.0, or eNpHr3.1 the ligand is yellow light (i.e., a wavelength of light from about 565 nm to about 590 nm) When the light sensitive protein is GtR3, ChR2, or CIVI the ligand is blue light (i.e., a wavelength of light from about 445 nm to about 520 nm). When the light sensitive protein is VChR1, or C1V1 the ligand is red light (i.e., a wavelength of light from about 625 nm to about 740 nm).

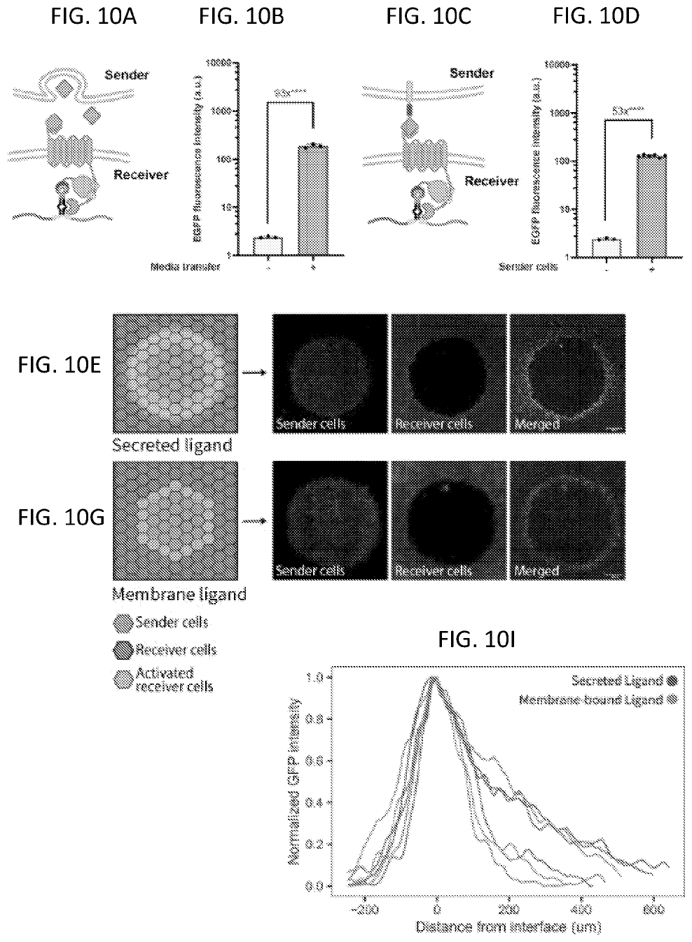

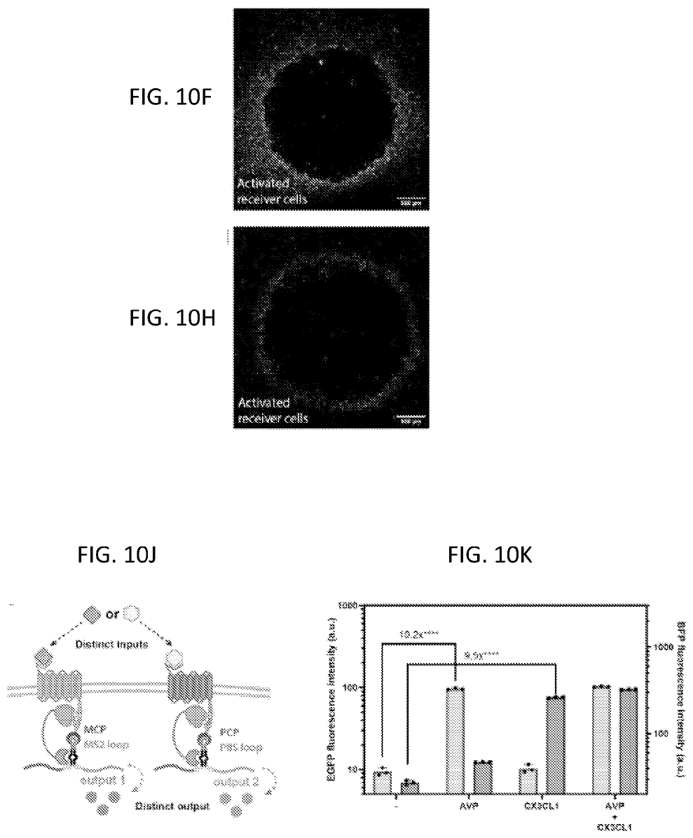

In some embodiments, the cell of the cell associated LIDAR mediated expression system contains more than one LIDAR mediated expression system. In some embodiments, the cell of the cell associated LIDAR mediated expression system contains two or more LIDAR mediated expression systems. In some embodiments, the two or more LIDAR mediated expression systems are according to FIG. 10J. In some embodiments, when there are two or more LIDAR mediated expression systems, the stem loop binding protein of the first LIDAR mediated expression system is different from the stem loop binding protein of the second LIDAR mediated expression system. For instance, the stem loop binding of the first LIDAR mediated expression system may be an MS2 binding protein and the stem loop binding of the second LIDAR mediated expression system may be a PP7 binding protein.

The methods disclosed herein utilize an output RNA, a stem loop binding protein and an RNA editing protein. The output RNA has an ADAR responsive site and an output polypeptide wherein the ADAR responsive site has a stem loop sequence and a stop codon. The stem loop binding protein has a stem loop binding domain and a dimerization domain. The RNA editing protein has an RNA editing domain and a dimerization domain. The stem loop binding protein binds to the stem loop within the ADAR responsive site. In the presence of the ligand, the dimerization domain of stem loop binding protein dimerizes with the dimerization domain of the RNA editing protein thereby bringing the RNA editing domain of the RNA editing protein into close proximity to the ADAR responsive site. The RNA editing domain then edits the stop codon to a non-stop codon thereby permitting the expression of the RNA coding sequence of the output polypeptide.

In some embodiments, the output RNA includes the following: (i) a first nucleotide sequence including an ADAR responsive site; (ii) a second nucleotide sequence encoding a cleavage domain, and (iii) a third nucleotide sequence encoding an output polypeptide.

In some embodiments, the output RNA includes the following: (i) a first nucleotide sequence including an ADAR responsive site; (ii) a second nucleotide sequence encoding a cleavage domain, (iii) a third nucleotide sequence encoding an output polypeptide, and (iv) a fourth nucleotide sequence encoding a marker protein wherein the fourth nucleotide sequence precedes the first nucleotide sequence.

In some embodiments, the output RNA includes the following: (i) a first nucleotide sequence including an ADAR responsive site; (ii) a second nucleotide sequence encoding a cleavage domain, (iii) a third nucleotide sequence encoding an output polypeptide, (iv) a fourth nucleotide sequence encoding a marker protein wherein the fourth nucleotide sequence precedes the first nucleotide sequence, and (v) a fifth nucleotide sequence encoding a second cleavage domain wherein the fifth nucleotide sequence precedes the first nucleotide sequence and is after the fourth nucleotide sequence.

In some embodiments, the output RNA has the first to third, fourth or fifth nucleotide in (i)-(v) order unless otherwise stated (i.e., wherein the fifth nucleotide sequence precedes the first nucleotide sequence and is after the fourth nucleotide sequence). In certain embodiments, the output RNA has the first nucleotide sequence to the third, fourth or fifth nucleotide sequences in order (i.e., the first nucleotide sequence is before the second nucleotide sequence which is before the third nucleotide sequence which is before the fourth nucleotide sequence which is before the fifth nucleotide sequence). In some embodiments, the output RNA has the first nucleotide sequence to the third, fourth or fifth nucleotide sequence that are not in order (i.e., the first nucleotide sequence is not before the fifth nucleotide sequence).

The output RNA of the present disclosure includes a first nucleotide sequence including an ADAR responsive site which is followed by a nucleotide sequence encoding an output polypeptide. In some embodiments, the ADAR responsive site includes a stem loop sequence including one or more stop codons. In some embodiments, the stem loop further includes at least 1 base that is mismatched with a sequence within the stem loop opposite the stop codon. In the presence of an ADAR protein or variant thereof, the ADAR protein then edits the adenosine base within the stop codon(s) of the output RNA to an inosine base. This editing removes the stop codon(s) which then allows the output polypeptide to be produced from the output RNA in the cell associated LIDAR mediated expression system.

When the output RNA has a nucleotide sequence containing a stem loop sequence containing a stop codon, any stem loop sequence may be used. In some embodiments, the stem loop is selected from the group of a 5-HT2CR stem loop, a Neill stem loop, an Azin1 stem loop, a BDF2 stem loop, a hGIL1 stem loop, a pre-miRNA-142 stem loop, a PP7 stem loop, and a GluR-B stem loop. Stem loops that are substrates of the RNA editing protein (i.e., an ADAR protein or variant thereof) have been described in, for example, in Thomas et al. (Bioessays. 2017 April; 39(4):10.1002/bies.201600187) and Pokharel et al. (ACS Chem Biol. 2006 Dec. 15; 1(12):761-5) each of which are specifically incorporated by reference herein. In some embodiments, the stem loop sequence has one or more, two or more, three or more, four or more, or five or more MS2 sequence repeatsMS2 repeats have been described in the art, for example, by Rodriques et al. (Nat Biotechnol. 2021 March; 39(3):320-325) which is specifically incorporated by reference. In some embodiments, the stem loop sequence has one or more, two or more, three or more, four or more, or five or more PP7 sequence repeats. PP7 repeats have been described in the art, for example, by Heinrich et al. (RNA. 2017 February; 23(2):134-141) which is specifically incorporated by reference.

The output RNA of the present disclosure contains a first nucleotide sequence containing an ADAR responsive site which is followed by a nucleotide sequence encoding an output polypeptide. In some embodiments, the ADAR responsive site contains a sensor nucleotide sequence that is reverse complementary to a target RNA, wherein the sensor nucleotide sequence contains one or more stop codons and a stem loop sequence. In embodiments where the ADAR responsive site contains a sensor nucleotide sequence, the cell associated LIDAR mediated expression system further contains a target RNA. In some embodiments, the sensor nucleotide sequence further contains at least 1 base that is mismatched with the target RNA sequence and a stem loop sequence. In the presence of the target RNA, the sensor nucleotide sequence of the sensor RNA hybridizes to the target RNA thereby forming a double stranded RNA molecule containing one or more base mismatches within the stop codon(s) or elsewhere. An ADAR protein or variant thereof then edits the adenosine base within the stop codon(s) of the sensor RNA to an inosine base. This editing removes the stop codon(s) which then allows the output polypeptide to be produced from the sensor RNA within the cell associated LIDAR mediated expression system.

The target RNA may be any RNA. For example, the target RNA includes, without limitation. mRNA, long non-coding RNA, transfer RNA, ribosomal RNA, small RNAs such as microRNA, small interfering RNA, small nucleolar RNAs, etc. In some embodiments, the target RNA may differentially expressed in different tissues cell types, or cell states and the detecting of the target RNA may be used to identify tissue types, cell types or cell states. In some embodiments, the target RNA may be a genetic variant of gene. In these instances, the genetic variant may be predictive of a disease or susceptible to a disease such as an oncogenic mutation or a genetic variant associated with increased susceptibility to a pathogen. In some embodiments, the methods of the present disclosure may be used to detect point mutations that are associated with the development of a disease such as cancer, neurogenerative disease, an autoimmune disease, etc. In some embodiments, the methods of the present disclosure are capable of detecting small indels, SNPs, etc. In some embodiments, the methods of the present disclosure are capable of detecting and distinguishing copy number variants within and between biological samples. The target RNA may also be gene fusion which may be predictive of cancer in general or a specific type of cancer.

The ADAR responsive site of embodiments of the present disclosure may include any stop codon containing an adenosine residue. For example, the stop codon of the sensor nucleotide sequence may be UAG, UAA, or UGA.

The sensor nucleotide sequence of the present disclosure may be reverse complementary to any region of the target RNA. In certain embodiments, the sensor nucleotide sequence is reverse complementary to the 3′ UTR of the target RNA. In some embodiments, the sensor nucleotide sequence is reverse complementary to the 5′ UTR of the target RNA. In certain embodiments, the sensor nucleotide sequence is reverse complementary to the coding sequence of the target RNA. In some embodiments, the sensor nucleotide sequence is reverse complementary to two separate non-contiguous regions of the same target RNA. For instance, the sensor nucleotide sequence may be reverse complementary to two separate regions of the 5′ UTR of the target RNA, to two separate regions of the coding sequence of the target RNA, to two separate regions of the 5′ UTR of the target RNA, to a region in the 5′ UTR and a region in the coding sequence of the target RNA, to a region in the coding sequence and a region in the 3′ UTR of the target RNA, or to a region in the 5′ UTR and a region in the 3′ UTR of the target RNA. In some embodiments, the sensor RNA is reverse complimentary to two or more distinct target RNAs.

The sensor nucleotide sequence of the present disclosure may be any length determined necessary for sufficient specificity to the target RNA. For instance the sensor nucleotide sequence could be less than about 50 nucleotides, from about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

When the sensor nucleotide sequence is reverse complementary to two non-contiguous regions within a target RNA, the distance between the two non-contiguous regions of the target may be any length. For instance, the distance between the two non-contiguous regions of the target may be less than about 50 nucleotides, from about 50 to 60, about 60 to 70, about 70 to 80, about 80 to 90, about 90 to 100, about 100 to 150, about 150 to 200, about 200 to 250, about 250 to 300, about 300 to 350, about 350 to 400, about 400 to 450, about 450 to 500 or greater than 500 nucleotides.

When the sensor nucleotide sequence is reverse complimentary to two non-contiguous regions within the target RNA, the nucleotide sequence of the sensor nucleotide that is reverse complementary to the first region of the two non-contiguous regions with the target RNA may be any length. For instance, the nucleotide sequence of the sensor nucleotide that is reverse complementary to the first region of the two non-contiguous regions may be less than about 20 nucleotides, from about 20 to 30, about 30 to 40, about 40 to 50, about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

When the sensor nucleotide sequence is reverse complimentary to two non-contiguous regions with the target RNA, the nucleotide sequence of the sensor nucleotide that is reverse complementary to the second region of the two non-contiguous regions with the target RNA may be any length. For instance, the nucleotide sequence of the sensor nucleotide that is reverse complementary to the second region of the two non-contiguous regions may be less than about 20 nucleotides, from about 20 to 30, about 30 to 40, about 40 to 50, about 50 to 60, about 60 to 70, about 70 to 80, about 80 to 90, about 90 to 100, about 100 to 110, about 110 to 120, about 120 to 130, about 130 to 140, about 140 to 150, about 150 to 160, about 160 to 170, about 170 to 180, about 180 to 190, about 190 to 200, about 200 to 210, about 210 to 220, about 220 to 230, about 230 to 240, about 240 to 250, about 250 to 260, about 260 to 270, about 270 to 280, about 280 to 290, about 290 to 300, about 300 to 310, about 310 to 320, about 320 to 330, about 330 to 340, about 340 to 350, about 350 to 360, about 360 to 370, about 370 to 380, about 380 to 390, about 390 to 400, about 400 to 410, about 410 to 420, about 420 to 430, about 430 to 440, about 440 to 450, about 450 to 460, about 460 to 470, about 470 to 480, about 480 to 490, about 490 to 500 or greater than 500 nucleotides in length.

The sensor nucleotide sequence of the present disclosure may include any stop codon having an adenosine residue. For example, the stop codon of the sensor nucleotide sequence may be UAG. UAA, or UGA.

In certain embodiments, the output RNA has a nucleotide sequence that encodes a cleavage domain. Cleavage domains that find use in the present disclosure include without limitation, a HIV-1 protease cleavage domain, a TEV cleavage domain, a preScission protease cleavage domain, a HCV protease cleavage domain, a RecA cleavage domain, a human kallikrein (KLK) cleavage domain, a human enterokinase cleavage domain, a human thrombin cleavage domain, a human matrix metalloprotease (MMP) cleavage domain, a human urokinase-type plasminogen activator receptor (uPAR) cleavage domain, a human plasmin cleavage domain, a human cathepsin cleavage domain, a self-cleaving domain, etc. When a self-cleaving domain is used then the self-cleaving domain may be a 2A self-cleaving domain. 2A self-cleaving domains that find use in the present disclosure include T2A, P2A, E2A and F2A which are described in Szymczak-Workman et al. (Cold Spring Harb Protoc. 2012 Feb. 1; 2012(2):199-204). In some embodiments, the output RNA has a cleavage domain encoded by the second nucleotide sequence and a cleavage domain encoded by the fifth nucleotide sequence. When the output RNA has a first and a second cleavage domain, the cleavage domains may be of the same type or they may be of a different type. For instance, the first cleavage domain may be a P2A self-cleaving domain and the second cleavage domain may also be a P2A self-cleaving domain or the first cleavage domain may be a P2A self-cleaving domain and the second cleavage domain may be a T2A self-cleaving domain or any combination thereof.

The output polypeptide of the present disclosure may be any output polypeptide desired. Examples of the output polypeptide of the present disclosure include, without limitation, a fluorescent protein, a genomic modification protein, a transcription factor, a killing factor, a toxin, an antigen, a T cell receptor, an enzyme, a cytokine, a chemokine, a growth factor, a signaling peptide, a chimeric antigen receptor (CAR), a protease, etc. The output polypeptides may be secreted, transmembrane or membrane-tethered. When output polypeptides are to be trafficked to specific locations within the cell associated system then the coding sequence of the output polypeptide is preceded by a nucleotide sequence encoding the appropriate signal peptide such as those described in Owji et al. (Eur J Cell Biol. 2018 August; 97(6):422-441).

When the output polypeptide is a genomic modification protein, the genomic modification proteins may include, without limitation, CRE recombinase or variants thereof, meganucleases or variants thereof. Zinc-finger nucleases or variants thereof, CRISPR/Cas-9 nuclease or variants thereof. TAL effector nucleases or variants thereof, etc.

When the output polypeptide is a transcription factor, the transcription factor may include, without limitation, jun, fos, max, mad, serum response factor (SRF), AP-1, AP2, myb, MyoD, myogenin. ETS-box containing proteins. TFE3, E2F, ATF1, ATF2, ATF3, ATF4, ZF5, NFAT, CREB, 5 HNF4, C/EBP, SP1, CCAAT-box binding proteins, interferon regulation factor (IRF-1), Wilms tumor protein, ETS-binding protein, STAT, dead Cas9 fused to an activation or repression domain, a Gal4 DNA binding domain fused to an activation or a repression domain, GATA-box binding proteins, e.g., GAT A-3, and the forkhead family of winged helix proteins.

When the output polypeptide is a cytokine, the cytokine may include, without limitation IL-1-like. IL-1α·IL-1β, IL-1RA, IL-18, CD132, IL-2, IL-4, IL-7, IL-9, IL-13, CD1243, 132, IL-15, CD131, IL-3, IL-5, GM-CSF, IL-6-like, IL-6, IL-11, G-CSF, IL-12, LIF, OSM, IL-10-like, IL-10, IL-20, IL-14, IL-16, IL-17. IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF, MSP, etc.

When the output polypeptide is a chemokine, the chemokine may include, without limitation XCL1, XCL2, CCL1, CCL2, CCL3, CCL4, CCL5, CCL7, CCL8, CCL11, CCL13, CCL14, CCL15, CCL16. CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, etc.

When the output polypeptide is a killing factor, the killing factor may include, without limitation, tumor necrosis factor alpha (TNFa), Fas ligang (FasL), a human Bcl-2-associated X protein (hBax), a caspase such as caspase 1, caspase 2, caspase 3, caspase 4, caspase 5, caspase 6, caspase 7, caspase 8, caspase 9, caspase 10, caspase 11, caspase 12, caspase 13 or a variant thereof, etc.

When the output polypeptide is a growth factor, the growth factor of may include, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angioproteinetins, angiostatin, granulocyte colony stimulating factor (GCSF), erythroproteinetin (EPO), connective tissue growth factor (CTGF), basic fibroblast growth factor (bFGF), acidic fibroblast growth factor (aFGF), epidermal growth factor (EGF), transforming growth factor .alpha. (TGFa), platelet-derived growth factor (PDGF), insulin growth factors I and II (IGF-1 and IGF-11), any one of the transforming growth factor 13-superfamily, including TGFI3, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In certain embodiments, when the output polypeptide is a CAR, the extracellular binding domain of the CAR has a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). Suitable CAR extracellular binding domains include those described in Labanieh et al. (2018 Nature Biomedical Engineering 2:377-391) which is specifically incorporated by reference herein. In some embodiments, the extracellular binding domain of the CAR is a single-chain version (e.g., an scFv version) of an antibody approved by the United States Food and Drug Administration and/or the European Medicines Agency (EMA) for use as a therapeutic antibody, e.g., for inducing antibody-dependent cellular cytotoxicity (ADCC) of certain disease-associated cells in a patient, etc. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab. Enfortumab. Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab. Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

When the output polypeptide is a protease, the protease is generally a protease that is not endogenous to the cell or organism to which the cell is in. The protease includes, without limitation, a Tobacco Etch Virus (TEV) protease, a plum pox virus protease (PPVp), a soybean mosaic virus protease (SbMVp), a sunflower mild mosaic virus protease (SuMMVp), a tobacco vein mottling virus protease (TVMVp), a West Nile virus protease (WNVp), a HIV-1 protease, a HCV protease, etc.

When the output polypeptide is a protease, the protease may cleave a site in a secondary protein that activates or deactivates the secondary protein. In some embodiments, the protease deactivates the secondary protein. In some embodiments, the protease activates the secondary protein. When the protease activates the secondary protein, the protease may cleave a site in a domain that promotes the degradation of the secondary protein thereby stopping the degradation of the secondary protein. The protease may also cleave a site in a domain that retains the secondary protein in a specific location thereby allowing the secondary protein to move to another location. In some embodiments, the secondary protein treats a discase or a condition. When the protease deactivates the secondary protein, the protease may cleave a site in a domain that contributes to the activity of the secondary protein thereby stopping, slowing or altering the activity of the secondary protein. The protease may also cleave a site in a domain that results in the dregradation of the secondary protein.

The output polypeptide may further have a tag to be used to detect the polypeptide following its production. For instance, the tag may include, without limitation, a fluorescent protein, e.g., green fluorescent protein (GFP), YFP, RFP, CFP, mCherry, tdTomato, and the like; a histidine tag, e.g., a 6xHis tag; a hemagglutinin (HA) tag; a FLAG tag; a Myc tag; and the like.

A marker protein of the present disclosure may be any marker protein that is useful for the detection of the presence of an output RNA. For instance, the marker protein may be a fluorescent protein or a luminescent protein. Non-limiting examples of useful fluorescent proteins include but are not limited to GFP, EBFP, Azurite, Cerulean, mCFP, Turquoise, ECFP, mKeima-Red, TagCFP, AmCyan, mTFP, TurboGFP, TagGFP, EGFP, TagYFP, EYFP, Topaz, Venus, mCitrine, TurboYFP, mOrange, TurboRFP, tdTomato, TagRFP, dsRed2, mRFP, mCherry, mPlum mRaspberry, mScarlet, etc. Examples of luminescent proteins, include without limitation, Cypridinia luciferase, Gaussia luciferase, Renilla luciferase, Phontinus luciferase, Luciola luciferase, Pyrophorus luciferase, Phrixothrix luciferase, etc. In some embodiments, the marker protein may be the first half of the output polypeptide. In these embodiments, the sequence encoding the marker protein produces the first half of the output which may be non-functional without the second half of the output polypeptide in the absence of the target RNA. In the presence of the target RNA, the second half of the output polypeptide is produced. When the second half of the output polypeptide is produced in the presence of the first half of the output polypeptide, the two halves are then able to form a functional output polypeptide. In these embodiments, the first half of the output polypeptide is the N-terminus of the output polypeptide and the second half of the output polypeptide is the C-terminus of the output polypeptide.

Additional designs of output RNAs are known in the art and have been described in, for example, international patent application PCT/US23/63245 which is specifically incorporated by reference herein.

The methods disclosed herein employ the use of a stem loop binding protein. The stem loop binding proteins of the present disclosure contain a stem loop binding domain and a dimerization domain joined by a linker. In some embodiments, the stem loop binding domain binds to a specific sequence within a stem loop sequence of the output RNA. In some embodiments, the stem loop binding domain binds to the MS2 sequence within the stem loop sequence. In some embodiments, the stem loop binding domain contains one or more, two or more, three or more, four or more, or five or more MS2 binding proteins. In some embodiments, the stem loop binding domain binds to the PP7 sequence within the stem loop sequence. In some embodiments, the stem loop binding domain contains one or more, two or more, three or more, four or more, or five or more PP7 binding proteins. In some embodiments, the stem loop binding domain binds to a TAR DNA element. TAR DNA elements are well known in the art, for example, as described by Rauch et al. (Cell. 2019 Jun. 27; 178(1):122-134.e12) which is specifically incorporated by reference herein. When the stem loop binding domain binds to TAR DNA elements, then the stem loop binding domain contains one or more, two or more, three or more, four or more, or five or more human hairpin-binding protein U1As (TBP6.7). In some embodiments, the stem loop binding domain contains one or more, two or more, three or more, four or more, or five or more stem loop binding proteins (SLBP). In some embodiments, the stem loop binding domain contains one or more, two or more, three or more, four or more, or five or more PP7 bacteriophage coat proteins.

The stem loop binding protein of the present disclosure also contains a dimerization domain. The dimerization domain may be any dimerization domain that is capable of dimerizing with the dimerization domain of the RNA editing protein. The dimerization domain of the stem loop binding protein may bind indirectly or directly to the dimerization domain of the RNA editing protein. When the dimerization domain of the stem loop binding protein binds indirectly to the dimerization domain of the RNA editing protein then the dimerization of both of the dimerization domains may be mediated by the ligand wherein the dimerization domain of the stem loop binding protein and the dimerization domain of the RNA editing protein both directly bind to the ligand. When dimerization is mediated by the ligand, the dimerization domain may include, without limitation, a natural binding domain, an engineered binding domain, FRB, FKBP, IL-2Rb-ec, IL-2Rg-ec, IL-10Ra-ec, IL-10Rb-ec, a receptor tyrosine kinase (RTK) or a fragment thereof, an antibody or a fragment thereof that specifically binds to the ligand, etc. When the dimerization domain is an RTK, then the RTK may be selected from any of the RTKs disclosed in Table 1.

TABLE 1
RTK families, RTKs and their cognate ligands.

| RTK Family | Individual RTK | Ligands |
|---|---|---|
| ErbB | EGFR/HER1, ERBB2/HER2, | EREG, EGF, TGFa, HB-EGF, BTC, |
| | ERBB3/HER3, ERBB4/HER4 | EPGN, AREG, NGR1, NRG2, |
| | | NGR3, NRG4, Neuroglycan C, |
| | | Tomoregulin |
| Ins | INSR, IGF1R, INSRR | Insulin, InslGF1, InslGF2, HASF, H⁺ |
| PDGF | PDGFRα, PDGFRβ, CSF1R, KIT, | PDGF A, PDGF B, PDGF C, PDGF |
| | FLT3L | D, SCF, Flt3L, CSF-1, IL-34 |
| VEGF | VEGFR1, VEGFR2, VEGFR3 | PIGF, VEGF B, VEGF, C, VEGF D |
| FGF | FGFR1, FGFR2, FGFR3, FGFR3 | FGF1-14, FGF16-18, FGF20, FGF22, |
| | | Heparan Sulfate, FGF19, FGF21, |
| | | FGF23 |
| PTK7 | PTK7/CCK4 | Co-receptor for Wnt signaling |
| Trk | TRKA, TRKB, TRKC | NGF, BDNf, NT3, NT4, DEHA |
| Ror | ROR1, ROR2 | Wnt5a |
| MuSK | MuSK | Agrin |
| Met | MET/RON | HGF, MSP |
| Axl | AXL, MER, TYRO3 | Gas6, Protein S |
| Tie | TIE1, TIE2 | Ang1, Ang2, Ang3, Ang4 |
| Eph | EPHA1, EPHA2, EPHA3, EPHA4, | Ephrin A1, Ephrin A2, Ephrin A3, |
| | EPHA5, EPHA6, EPHB1, EPHB2, | Ephrin A4, Ephrin A5, Ephrin B1, |
| | EPHB3, EPHB4, EPHB5, EPHB6, | Ephrin B2, Ephrin B3, Ephrin B4, |
| | | Ephrin B5, Membrane-cleaved |
| | | Ephrin |
| Ret | RET | GDNF, Neuturin, Artemin, Persephin, |
| | | GDF15 |
| Ryk | RYK | Wnt, Wnt3a |
| DDR | DDR1, DDR2 | Collagen type I, type II, type III, type |
| | | IV, type X |
| Ros | ROS | unknown |
| ALK | ALK, LTK | ALKAL1, ALKAL2 |
| STYK | STYK1 | unknown |

When the dimerization domain of the stem loop binding protein directly interacts with the dimerization domain of the RNA editing protein, the dimerization domain may include, without limitation, a G-protein coupled receptor (GPCR), a polypeptide that binds to a GPCR only after it binds to the ligand, a chemokine receptor, an opioid receptor, an insulin-like growth factor receptor, b-arrestin, Shc, visual arrestin, a receptor tyrosine kinase (RTK) or a fragment thereof, and an antibody or a fragment thereof that specifically binds to the dimerization domain of the RNA editing protein, etc. When the dimerization domain is a GPCR, the GPCR may be selected from Table 2. In some embodiments, the dimerization domain of the stem loop binding protein has a V2 tail. In some embodiments, the dimerization domain of the stem loop binding protein is post-translationally modified after binding the ligand. In some embodiments, the dimerization domain of the RNA editing protein only binds to the dimerization domain of the stem loop binding protein when the dimerization domain of the stem loop binding protein is bound to the ligand.

TABLE 2
GPCRs and their cognate ligands.

| GPCR | Ligand |
|---|---|
| 5HT1A | 5-Hydroxytryptamine |
| 5HT1B | 5-Hydroxytryptamine, Tryptamine, 5-Ht- |
| | Moduline |
| 5HT1D | 5-Hydroxytryptamine, 5-Ht-Moduline |
| 5HT1E | 5-Hydroxytryptamine, Tryptamine |
| 5HT1F | 5-Hydroxytryptamine |
| 5HT2A | 5-Hydroxytryptamine, Tryptamine |
| 5HT2B | 5-Hydroxytryptamine |
| 5HT2C | 5-Hydroxytryptamine |
| 5HT4R | 5-Hydroxytryptamine |
| 5HT5A | 5-Hydroxytryptamine |
| 5HT6R | 5-Hydroxytryptamine |
| 5HT7R | 5-Hydroxytryptamine |
| AA1R | Adenosine |
| AA2AR | Adenosine |
| AA2BR | Adenosine |
| AA3R | Adenosine |
| ACKR1 | Ccl17, Ccl14, Ccl2, Cxcl8, Cxcl11, Ccl11, |
| | Ccl5, Ccl7, Cxcl5, Cxcl6 |
| ACKR2 | Ccl11, Ccl2, Ccl5, Ccl3, Ccl14, Ccl22, Ccl7, |
| | Ccl8, Ccl17, Ccl13, Ccl4 |
| ACKR3 | Cxcl12, Cxcl11 |
| ACKR4 | Ccl25, Ccl19, Ccl21 |
| ACM1 | Acetylcholine |
| ACM2 | Acetylcholine |
| ACM3 | Acetylcholine |
| ACM4 | Acetylcholine |
| ACM5 | Acetylcholine |
| ACTHR | Acth |
| ADA1A | (−)-Noradrenaline, (−)-Adrenaline |
| ADA1B | (−)-Adrenaline, (−)-Noradrenaline |
| ADA1D | (−)-Adrenaline, (−)-Noradrenaline |
| ADA2A | (−)-Adrenaline, (−)-Noradrenaline |
| ADA2B | (−)-Adrenaline, (−)-Noradrenaline |
| ADA2C | (−)-Adrenaline, (−)-Noradrenaline |
| ADRB1 | (−)-Adrenaline, (−)-Noradrenaline, |
| | Noradrenaline |
| ADRB2 | Zn2+, (−)-Noradrenaline, Noradrenaline, (−)- |
| | Adrenaline |
| ADRB3 | (−)-Adrenaline, (+)-Adrenaline, (−)- |
| | Noradrenaline, (−)-Adrenaline |
| AGRB1 | Phosphatidylserine |
| AGRL1 | Lasso D |
| AGTR1 | Angiotensin A, Angiotensin II, Angiotensin IV, |
| | Angiotensin III |
| AGTR2 | Angiotensin-(1-7), Angiotensin III, Angiotensin |
| | II |
| APJ | Pyr1]Apelin-13, Apelin Receptor Early |
| | Endogenous Ligand, Apelin-13, |
| | Elabela/Toddler-11, Elabela/Toddler-32, |
| | Elabela/Toddler-21, Apelin-36, Apelin-17, |
| | Apelin Receptor Early Endogenous Ligand, |
| | Apelin-36 |
| BKRB1 | Kallidin, [Des-Arg9]Bradykinin, [Des- |
| | Arg10]Kallidin, Lys-[Hyp3]-Bradykinin, T- |
| | Kinin, Bradykinin, [Hyp3]Bradykinin |
| BKRB2 | Kallidin, Bradykinin, T-Kinin, [Des- |
| | Arg9]Bradykinin, [Hyp3]Bradykinin, [Des- |
| | Arg10]Kallidin, Lys-[Hyp3]-Bradykinin |
| C3AR | C5a, C3a, C5a, C3a, C3a, C5a |
| C5AR1 | C3a, C5a, C3a, C5a Des-Arg, C5a, C5a, |
| | Ribosomal Protein S19, C3a |
| C5AR2 | C5a, C5a Des-Arg |
| CALCR | Calcitonin, Amylin, Adrenomedullin, |
| | Calcitonin, Adrenomedullin 2/Intermedin, |
| | Amylin, Calcitonin (Salmon) |
| CASR | Ca2+, Mg2+, L-Tryptophan, Spermine |
| CCKAR | Cck-33, Gastrin-17, Gastrin-17, Desulfated |
| | Cholecystokinin-8, Cck-4, Cck-58, Cck-33, |
| | Cck-8, Cck-39, Gastrin-17, Cck-33 |
| CCR1 | Ccl23, Ccl7, Ccl8, Ccl4, Ccl4, Ccl3, Ccl4, |
| | Ccl3, Ccl5, Ccl13, Ccl3, Ccl7, Ccl16, Ccl15, |
| | Ccl5, Ccl7, Ccl8, Ccl14 |
| CCR10 | Ccl27, Ccl27 |
| CCR2 | Ccl16, Ccl26, Ccl2, Ccl11, Ccl11, Ccl2, Ccl7, |
| | Ccl7, Ccl24, Ccl7, Ccl2, Ccl11, Ccl13, Ccl8, |
| | Ccl8 |
| CCR3 | Ccl11, Ccl7, Ccl24, Cxcl10, Ccl26, Cxcl11, |
| | Cxcl10, Ccl11, Ccl7, Ccl13, Ccl28, Ccl24, |
| | Ccl15, Cxcl9, Cxcl10, Ccl5, Cxcl9, Ccl5, |
| | Cxcl11, Ccl8, Ccl28, Ccl11, Ccl2, Ccl8, Ccl2, |
| | Ccl7, Ccl2 |
| CCR4 | Ccl17, Ccl22, Ccl22 |
| CCR5 | Ccl4, Ccl3, Ccl11, Ccl5, Ccl7, Ccl11, Ccl3, |
| | Ccl4, Ccl16, Ccl2, Ccl8, Ccl5, Ccl2, Ccl14, |
| | Ccl8, Ccl11, Ccl4, Ccl7, Ccl7, Ccl13, Ccl2, |
| | Ccl3 |
| CCR6 | Ccl20, Ccl20, Beta-Defensin 4a, Ccl20 |
| CCR7 | Ccl21b, Ccl19, Ccl21 |
| CCR8 | Ccl1, Ccl1, Ccl8 |
| CCR9 | Ccl25 |
| CCRL2 | Ccl19, Chemerin C-Terminal Peptide |
| CLTR1 | Ltd4, Lte4, Ltc4 |
| CLTR2 | Ltd4, Ltc4, Lte4 |
| CML1 | 18r-Hepe, Chemerin, Epa, Resolvin E1, |
| | Chemerin C-Terminal Peptide |
| CML2 | Chemerin |
| CNR1 | Anandamide, 2-Arachidonoylglycerol |
| CNR2 | Anandamide, 2-Arachidonoylglycerol |
| CRFR1 | Urocortin 1, Corticotrophin-Releasing |
| | Hormone, Urocortin 1, Urocortin 2 |
| CRFR2 | Urocortin 1, Corticotrophin-Releasing |
| | Hormone, Urocortin 2, Urocortin 3 |
| CX3C1 | Cx3Cl1 |
| CXCR1 | Cxcl8, Cytokine Domain Of Tyrosyl Trna |
| | Synthetase, Cxcl6 |
| CXCR2 | Cxcl1, Cxcl2, Cxcl8, Cxcl3, Cxcl7, Cxcl5, |
| | Cxcl6 |
| CXCR3 | Cxcl11, Cxcl10, Cxcl10, Ccl5, Ccl7, Ccl13, |
| | Ccl11, Cxcl9, Cxcl9, Cxcl12alpha, Cxcl11, |
| | Cxcl10, Ccl19, Ccl20 |
| CXCR4 | Cxcl12phi, Cxcl12delta, Cxcl12alpha, |
| | Cxcl12beta, Cxcl12, Cxcl12gama, |
| | Cxcl12epsilon |
| CXCR5 | Cxcl13 |
| CXCR6 | Cxcl16 |
| DRD1 | Noradrenaline, Dopamine, (−)-Noradrenaline, 5- |
| | Hydroxytryptamine |
| DRD2 | Dopamine |
| DRD3 | Dopamine |
| DRD4 | Dopamine |
| DRD5 | 5-5-Hydroxytryptamine, (−)-Noradrenaline, |
| | Noradrenaline, Dopamine |
| EDNRA | Endothelin-1, Endothelin-2, Endothelin-3 |
| EDNRB | Endothelin-1, Endothelin-2, Endothelin-3 |
| FFAR1 | Docosahexaenoic Acid, Oleic Acid, Myristic |
| | Acid, alpha-Linolenic Acid |
| FFAR2 | Butyric Acid, Acetic Acid, Trans-2- |
| | Methylcrotonic Acid, Propanoic Acid, 1- |
| | Methylcyclopropanecarboxylic Acid |
| FFAR3 | Propanoic Acid, 1- |
| | Methylcyclopropanecarboxylic Acid, Butyric |
| | Acid |
| FFAR4 | Linoleic Acid, aplha-Linolenic Acid, Oleic |
| | Acid, Myristic Acid |
| FPR1 | Cathepsin G, Fmet-Leu-Phe, Annexin I, |
| | Spinorphin |
| FPR2 | Resolvin D1, Prp106-126, Annexin I, Serum |
| | Amyloid A, Lxa4, Ll-37, Ltd4, Aspirin- |
| | Triggered Resolvin D1, Fmet-Leu-Phe, 15- |
| | Deoxy-Lxa4, Humanin, Aspirin Triggered |
| | Lipoxin A4, Ltc4, Atla2 |
| FPR3 | F2l, Humanin, Annexin I-(2-26), F2l |
| FSHR | Fsh |
| FZD1 | Wnt-7b, Wnt-1, Wnt-5a, Wnt-2, Wnt-3a |
| FZD2 | Wnt-5a |
| FZD6 | Wnt-3a, Wnt-4, Wnt-5a |
| G37L1 | Prosaptide |
| GABR1 | Gaba, Galanin, Galanin-Like Peptide |
| GALR2 | Galanin, Spexin-1, Galanin-Like Peptide |
| GALR3 | Galanin, Galanin-Like Peptide, Spexin-1 |
| GASR | Gastrin-17, Cck-8, Gastrin-34, Cck-4, Cck-33, |
| | Desulfated Gastrin-71, Gastrin-71, Desulfated |
| | Gastrin-17, Cck-39, Cck-33, Gastrin-14, |
| | Desulfated Gastrin-14, Gastrin-17, Desulfated |
| | Gastrin-34, Desulfated Cholecystokinin-8, Cck- |
| | 58, Cck-33 |
| GHRHR | Ghrh |
| GHSR | Ghrelin, [Des-Gln14]Ghrelin |
| GIPR | Gastric Inhibitory Polypeptide |
| GLP1R | Glucagon-Like Peptide 1-(7-36) Amide, |
| | Glucagon-Like Peptide 1-(7-37), Glucagon |
| GLP2R | Glucagon-Like Peptide 2, Glucagon-Like |
| | Peptide 2-(3-33) |
| GLR | Glucagon |
| GNRHR | Gnrh Ii, Gnrh I |
| GP107 | Neuronostatin |
| GP119 | N-Oleoylethanolamide, Anandamide, Sea, N- |
| | Palmitoylethanolamine |
| GP132 | 9-Hydroxyoctadecadienoic Acid |
| GP139 | L-Phenylalanine, L-Tryptophan |
| GP143 | Levodopa |
| GP174 | Lysophosphatidylserine |
| GP183 | 7beta, 25-Dihydroxycholesterol, 7beta, 27- |
| | Dihydroxycholesterol, 7alpha- |
| | Hydroxycholesterol, 27-Hydroxycholesterol, |
| | 7beta-Hydroxycholesterol, 7alpha, 25- |
| | Dihydroxycholesterol, 25-Hydroxycholesterol, |
| | 7alpha, 27-Dihydroxycholesterol |
| GPBAR | Deoxycholic Acid, Chenodeoxycholic Acid, |
| | Cholic Acid, Lithocholic Acid |
| GPC6A | L-Serine, L-Ornithine, L-Alanine, L-Lysine, L- |
| | Arginine, L-Citrulline, Glycine, L-Glutamine |
| GPER1 | 17beta-Estradiol |
| GPR12 | Udp-Glucose, Ltc4, Ltd4, Udp-Galactose, Atp, |
| | Uridine Diphosphate, Lte4 |
| GPR18 | N-Arachidonoylglycine |
| GPR3 | Sphingosine 1-Phosphate |
| GPR31 | 12s-Hete |
| GPR32 | Resolvin D1, Lxa4 |
| GPR34 | Lysophosphatidylserine |
| GPR35 | Kynurenic Acid, 2-Oleoyl-Lpa |
| GPR37 | Prosaptide |
| GPR39 | Obestatin, Zn2+, Obestatin |
| GPR55 | 2-Arachidonoylglycerolphosphoinositol, 2- |
| | Arachidonoylglycerol, N- |
| | Palmitoylethanolamine, |
| | Lysophosphatidylinositol, Anandamide |
| GPR6 | Sphingosine 1-Phosphate |
| GPR63 | Dioleoylphosphatidic Acid, Sphingosine 1- |
| | Phosphate, Dihydrosphingosine 1-Phosphate |
| GPR75 | Ccl5 |
| GPR87 | Lpa |
| GRM1 | L-Glutamic Acid |
| GRM2 | L-Glutamic Acid |
| GRM3 | Naag, L-Glutamic Acid |
| GRM4 | L-Glutamic Acid, L-Serine-O-Phosphate |
| GRM5 | L-Glutamic Acid |
| GRM6 | L-Glutamic Acid, L-Serine-O-Phosphate |
| GRM7 | L-Serine-O-Phosphate, L-Glutamic Acid |
| GRM8 | L-Serine-O-Phosphate, L-Glutamic Acid |
| GRPR | Gastrin-Releasing Peptide, Neuromedin |
| | B, Grp-(18-27), Neuromedin C, Grp-(18- |
| | 27), Gastrin Releasing Peptide(14-27) |
| | (Human) |
| HCAR1 | L-Lactic Acid |
| HCAR2 | Butyric Acid, beta-D-Hydroxybutyric |
| | Acid |
| HCAR3 | 3-Hydroxyoctanoic Acid |
| HRH1 | Histamine |
| HRH2 | Histamine |
| HRH3 | Histamine |
| HRH4 | Histamine, Ccl16 |
| KISSR | Kisspeptin-54, Kisspeptin-52, Kisspeptin- |
| | 13, Kisspeptin-10, Kisspeptin-52, |
| | Kisspeptin-14, Kisspeptin-10 |
| LGR4 | R-Spondin-1, R-Spondin-2, R-Spondin-3 |
| LGR5 | R-Spondin-1, R-Spondin-2, R-Spondin-3 |
| LGR6 | R-Spondin-1, R-Spondin-2, R-Spondin-3 |
| LPAR2 | Lpa, Farnesyl Diphosphate, Farnesyl |
| | Monophosphate |
| LPAR3 | Lpa, Farnesyl Monophosphate, Farnesyl |
| | Diphosphate |
| LPAR4 | Farnesyl Diphosphate, Lpa |
| LPAR5 | Lpa, Farnesyl Diphosphate, Farnesyl |
| | Monophosphate, N-Arachidonoylglycine |
| LPAR6 | Lpa |
| LSHR | Lh, Hcg, Lh, Lh |
| LT4R1 | 20-Hydroxy-Ltb4, Ltb4, 12r-Hete |
| LT4R2 | 12-Hydroxyheptadecatrienoic Acid, Ltb4, |
| | 15s-Hete, 12s-Hete, 12-Epi Ltb4, 12r- |
| | Hete, 20-Hydroxy-Ltb4, 12s-Hpete |
| MC3R | gamma-Msh, Acth, alpha-Msh, beta-Msh, |
| | Agouti-Related Protein, Acth, |
| MC4R | alpha-Msh, Agouti-Related Protein, Acth, |
| | beta-Msh, gamma-Msh |
| MC5R | gamma-Msh, Acth, beta-Msh, alpha-Msh, |
| | Acth, Agouti-Related Protein |
| MCHR1 | Melanin-Concentrating Hormone |
| MCHR2 | Melanin-Concentrating Hormone |
| MRGRD | beta-Alanine |
| MRGX1 | Bovine Adrenal Medulla Peptide 8-22 |
| MRGX2 | Pamp-20 |
| MSHR | Acth, beta-Msh, alpha-Msh, gamma- |
| | MSH |
| MTLR | Motilin |
| MTR1A | Melatonin |
| MTR1B | Melatonin |
| NK1R | Substance P, Neurokinin B, Neuropeptide |
| | K, Neuropeptide-gamma, Hemokinin 1, |
| | Neurokinin A |
| NK2R | Substance P, Neurokinin A, Neurokinin |
| | B, Hemokinin 1 |
| NK3R | Substance P, Neurokinin B, Neurokinin |
| | A, Hemokinin 1 |
| NMBR | Neuromedin B, Grp-(18-27), Gastrin |
| | Releasing Peptide(14-27) (Human), |
| | Gastrin-Releasing Peptide |
| NMUR1 | Neuromedin U-25, Neuromedin S-36, |
| | Neuromedin U-23, Neuromedin U-23, |
| | Neuromedin S-36, Neuromedin S-33 |
| NMUR2 | Neuromedin S-33, Neuromedin U-23, |
| | Neuromedin U-25, Neuromedin S-36, |
| | Neuromedin S-36 |
| NPBW1 | Neuropeptide W-30, Neuropeptide W-30, |
| | Des-Br-Neuropeptide B-23, Neuropeptide |
| | B-29, Neuropeptide B-29, Neuropeptide |
| | B-23, Des-Br-Neuropeptide B-29, |
| | Neuropeptide W-23, Neuropeptide B-23, |
| | Neuropeptide B-29, Neuropeptide W-23, |
| | Neuropeptide W-30, Neuropeptide B-23 |
| NPBW2 | Neuropeptide B-29, Neuropeptide B-29, |
| | Neuropeptide W-23, Neuropeptide B-23, |
| | Neuropeptide B-23, Neuropeptide B-29, |
| | Neuropeptide W-23, Neuropeptide W-30, |
| | Neuropeptide W-30, Neuropeptide W-30, |
| | Neuropeptide B-23 |
| NPFF1 | Neuropeptide Sf, Rfrp-1, Neuropeptide |
| | Af, Neuropeptide Af, Prrp-31, Qrfp43 |
| | (43rfa), Neuropeptide Sf, Neuropeptide |
| | Ff, Rfrp-3, Neuropeptide Af, |
| | Neuropeptide Sf |
| NPFF2 | Prrp-31, Neuropeptide Af, Qrfp43 |
| | (43rfa), Neuropeptide Ff, Neuropeptide |
| | Af, Neuropeptide Sf, Neuropeptide Af, |
| | Rfrp-1, Rfrp-3 |
| NPSR1 | Neuropeptide S |
| NPY1R | Neuropeptide Y, Peptide Yy |
| NPY2R | Pancreatic Polypeptide, Peptide Yy, |
| | Neuropeptide Y-(3-36), Pyy-(3-36), |
| | Neuropeptide Y |
| NPY4R | Neuropeptide Y, Peptide Yy, Pancreatic |
| | Polypeptide |
| NPY5R | Pancreatic Polypeptide, Pyy-(3-36), |
| | Peptide Yy, Neuropeptide Y |
| NTR1 | Large Neurotensin, Neuromedin N, Large |
| | Neuromedin N |
| NTR2 | Neuromedin N, Neurotensin |
| OPRD | Dynorphin A-(1-13), beta-Endorphin, |
| | [Leu]Enkephalin, Dynorphin B, |
| | Endomorphin-1, Dynorphin A, |
| | Dynorphin A-(1-8) |
| OPRK | alpha-Neoendorphin, Dynorphin A-(1-8), |
| | Dynorphin A-(1-13), beta-Endorphin, |
| | Dynorphin B, beta-Neoendorphin, |
| | Dynorphin A, Big Dynorphin |
| OPRM | beta-Endorphin, Endomorphin-1, |
| | Dynorphin B, Dynorphin A-(1-13), |
| | [Leu]Enkephalin, Dynorphin A-(1-8), |
| | Dynorphin A |
| OPRX | Nociceptin/Orphanin Fq |
| OX1R | Orexin-A, Orexin-A |
| OX2R | Orexin-A, Orexin-A |
| OXER1 | 5-Oxo-C20:3, 5-Oxo-15-Hete, 5-Oxo-Ete, |
| | 5s-Hete, 5-Oxo-20-Hete, 5s-Hpete, 5- |
| | Oxo-Ode, 5-Oxo-12-Hete |
| OXGR1 | alpha-Ketoglutaric Acid |
| OXYR | Oxytocin |
| OXYR | Vasopressin |
| P2RY1 | Adp, atp |
| P2RY2 | Atp, Uridine Triphosphate |
| P2RY4 | Atp, Uridine Triphosphate |
| P2RY6 | Uridine Triphosphate, Adp, Uridine |
| | Diphosphate |
| P2Y10 | Sphingosine 1-Phosphate, Lpa |
| P2Y11 | Atp, Adp, Uridine Triphosphate |
| P2Y12 | Atp |
| P2Y13 | Atp, Adp |
| P2Y14 | Uridine Diphosphate, Udp N-Acetyl- |
| | Glucosamine, Udp-Glucose, Udp- |
| | Glucuronic Acid, Udp-Galactose |
| PACR | Pacap-27, Pacap-38, Vip, Phv, Phi, Phm, |
| | Phv |
| PAR1 | Thrombin |
| PAR3 | Thrombin |
| PAR4 | Thrombin, Cathepsin G |
| PD2R | Pgd2, Pgj2, Pgf2alpha, Pgi2, Pge2, |
| | Thromboxane A2, Pge1 |
| PD2R2 | Pgi2, Pgd3, Pge2, Pgd2, Pgj2, |
| | Thromboxane A2, Pgf2alpha |
| PE2R1 | Pgf2alpha, Pge1, Thromboxane A2, |
| | Pgd2, Pge2, Pgi2 |
| PE2R2 | Pgf2alpha, Pgd2, Pge1, Pge2, |
| | Thromboxane A2, Pgi2 |
| PE2R3 | Pge2, Pgf2alpha, Pgd2, Thromboxane |
| | A2, Pge1, Pgi2 |
| PE2R4 | Pge2, Pgd2, Pgi2, Pge1, Pgf2alpha, |
| | Thromboxane A2 |
| PF2R | Pge2, Pgi2, Pgd2, Pgf2alpha, |
| | Thromboxane A2 |
| PI2R | Pge1, Pge2, Thromboxane A2, Pgd2, |
| | Pgf2alpha, Pgi2 |
| PKR1 | Prokineticin-2, Prokineticin-1, |
| | Prokineticin-2alpha |
| PKR2 | Prokineticin-2, Prokineticin-1, |
| | Prokineticin-2alpha |
| PRLHR | Pthrp, Prrp-20, Prrp-31, Prrp-20, |
| | Neuropeptide Y, Prrp-31 |
| PTAFR | Paf, Methylcarbamyl Paf |
| PTH1R | Pth, Pthrp-(1-36), Pthrp, Pth, Tip39, Pth |
| PTH2R | Tip39, Pthrp, Pth, Pthrp-(1-36), Pthrp-(1- |
| | 34) (Human) |
| QRFPR | Qrfp26 (26rfa), Qrfp43 (43rfa), Qrfp43 |
| RL3R1 | Relaxin-3, Relaxin-3 (B Chain), Relaxin, |
| | Insl5 |
| RL3R2 | Relaxin-3, Insl5, Relaxin-3 (B Chain) |
| RXFP1 | Relaxin-3, Relaxin, Relaxin-1 |
| RXFP2 | Relaxin-3, Relaxin, Relaxin-1, Insl3 |
| S1PR1 | Sphingosine 1-Phosphate, |
| | Sphingosylphosphorylcholine, |
| | Dihydrosphingosine 1-Phosphate |
| S1PR2 | Sphingosine 1-Phosphate, |
| | Sphingosylphosphorylcholine, |
| | Dihydrosphingosine 1-Phosphate |
| S1PR3 | Sphingosine 1-Phosphate, |
| | Dihydrosphingosine 1-Phosphate, |
| | Sphingosylphosphorylcholine |
| S1PR4 | Sphingosine 1-Phosphate, |
| | Dihydrosphingosine 1-Phosphate, |
| | Sphingosylphosphorylcholine |
| S1PR5 | Sphingosine 1-Phosphate, |
| | Dihydrosphingosine 1-Phosphate, |
| | Sphingosylphosphorylcholine |
| SCTR | Secretin |
| SCTR | Vip |
| SSR1 | Cst-17, Srif-14, Srif-28, Cortistatin-14 |
| SSR2 | Srif-28, Srif-14, Cortistatin-14, Cst-17 |
| SSR3 | Srif-28, Srif-14, Cst-17, Cortistatin-14 |
| SSR4 | Cortistatin-14, Srif-14, Srif-28, Cst-17 |
| SSR5 | Srif-14, Cst-17, Cortistatin-14, Srif-28 |
| SUCR1 | Succinic Acid, Maleic Acid |
| TA2R | Pgd2, Pge2, Thromboxane A2, Pgi2, |
| | Pgh2, Pgf2alpha |
| TAAR1 | Tyramine, beta-Phenylethylamine, 3- |
| | Iodothyronamine, Octopamine, |
| | Dopamine |
| TAAR2 | Tryptamine, beta-Phenylethylamine |
| TAAR3 | Isoamylamine |
| TSHR | Tsh |
| UR2R | Urotensin-Ii, Urotensin Ii-Related Peptide |
| V1AR | Vasopressin, Oxytocin |
| V1BR | Vasopressin, Oxytocin |
| V2R | Vasopressin, Oxytocin |
| VIPR1 | Vip, Phv, Secretin, Phm, Phi, Ghrh, Ghrh, |
| | Secretin, Ghrh, Pacap-38, Pacap-27, |
| | Secretin, Phi |
| VIPR2 | Vip, Phi, Pacap-27, Pacap-38, Ghrh, |
| | Ghrh, Secretin, Secretin, Phv, Ghrh, Phi, |
| | Secretin |
| XCR1 | Xcl1, Xcl2 |

The stem loop binding domain and the dimerization domain of the stem loop binding protein are joined by a linker. The linker may be any linker deemed useful. In some embodiments, the peptide linker has 2, 3, 4, 5, 6, 7, 8, 9, 10, 11, 12, 13, 14, 15, 16, 17, 18, 19, 20 or greater than 20 amino acids. In some embodiments, the peptide linker is between 1 to 5, 1 to 10, 1 to 15, 1 to 20, 5 to 10, 5 to 15 or 5 to 20 amino acids in length. In a preferred embodiment, the linker is 6 amino acids or less. Exemplary linkers include linear peptides having at least two amino acid residues such as Gly-Gly, Gly-Ala-Gly, Gly-Pro-Ala, Gly-Gly-Gly-Gly-Ser (SEQ ID NO: 1). Suitable linear peptides include poly glycine, polyserine, polyproline, polyalanine and oligopeptides consisting of alanyl and/or serinyl and/or prolinyl and/or glycyl amino acid residues. In some embodiments, the peptide linker has the amino acid sequence selected from the group consisting of Gly. (SEQ ID NO: 2), Glu9 (SEQ ID NO: 3), Ser, (SEQ ID NO: 4), Glys-Cys-Pro2-Cys (SEQ ID NO: 5), (Gly4-Ser); (SEQ ID NO: 6). Ser-Cys-Val-Pro-Leu-Met-Arg-Cys-Gly-Gly-Cys-Cys-Asn (SEQ ID NO: 7), Pro-Ser-Cys-Val-Pro-Leu-Met-Arg-Cys-Gly-Gly-Cys-Cys-Asn (SEQ ID NO: 8), Gly-Asp-Leu-Ile-Tyr-Arg-Asn-Gln-Lys (SEQ ID NO: 9), and Gly9-Pro-Ser-Cys-Val-Pro-Leu-Met-Arg-Cys-Gly-Gly-Cys-Cys-Asn (SEQ ID NO: 10). In a preferred embodiment, the linker is a Gly-Thr-Gly-Ala (SEQ ID NO: 11) linker.

The methods disclosed herein employ the use of an RNA editing protein. The RNA editing proteins of the present disclosure contain an RNA editing domain and a dimerization domain joined by a linker. In some embodiments, the RNA editing domain edits specific sequences within a stem loop sequence or a sequence near the stem loop sequence of the output RNA. In some embodiments, the RNA editing domain of the RNA editing protein contains an ADAR protein. In some embodiments, the ADAR protein is an ADAR1 or ADAR2 deaminase domain. In some embodiments, the ADAR1 or ADAR2 deaminase domain is a modified deaminase domain with enhanced editing activity. In some embodiments, the modified deaminase domain is a T501A deaminase domain variant. Modified deaminase domains have been described in the art, for example, by Katrekar et al. (Elife. 2022 Jan. 19; 11:e75555), Matthews et al. (Nat Struct Mol Biol. 2016 May; 23(5):426-33), Katrekar et al. (Nat Methods. 2019 March; 16(3):239-242), which are each specifically incorporated by reference herein.

The RNA editing protein of the present disclosure also contains a dimerization domain. The dimerization domain may be any dimerization domain that is capable of dimerizing with the dimerization domain of the stem loop binding protein. The dimerization domain of the RNA editing protein may bind indirectly or directly to the dimerization domain of the stem loop binding protein. When the dimerization domain of the RNA editing protein binds indirectly to the dimerization domain of the stem loop binding protein then the dimerization of both of the dimerization domains may be mediated by the ligand wherein the dimerization domain of the RNA editing protein and the dimerization domain of the stem loop binding protein both directly bind to the ligand. When dimerization is mediated by the ligand, the dimerization domain may include, without limitation, a natural binding domain, an engineered binding domain. FRB. FKBP, IL-2Rb-ec, IL-2Rg-ec, IL-10Ra-ec, IL-10Rb-ec, a receptor tyrosine kinase (RTK) or a fragment thereof, an antibody or a fragment thereof that specifically binds to the ligand, etc. When the dimerization domain is an RTK, then the RTK may be selected from any of the RTKs disclosed in Table 1.

When the dimerization domain of the RNA editing protein directly interacts with the dimerization domain of the stem loop binding protein, the dimerization domain may include, without limitation, a G-protein coupled receptor (GPCR), a polypeptide that binds to a GPCR only after it binds to the ligand, a chemokine receptor, an opioid receptor, an insulin-like growth factor receptor, b-arrestin, She, visual arrestin, a receptor tyrosine kinase (RTK) or a fragment thereof, and an antibody or a fragment thereof that specifically binds to the dimerization domain of the RNA editing protein, etc. When the dimerization domain is a GPCR, the GPCR may be selected from Table 2. In some embodiments, the dimerization domain of RNA editing protein has a V2 tail. In some embodiments, the dimerization domain of the RNA editing protein is post-translationally modified after binding the ligand. In some embodiments, the dimerization domain of the stem loop binding protein only binds to the dimerization domain of the RNA editing protein when the dimerization domain of the RNA editing protein is bound to the ligand.

The RNA editing domain and the dimerization domain of the RNA editing protein are joined by a linker. The linker may be any linker deemed useful. In some embodiments, the peptide linker has 2, 3, 4, 5, 6, 7, 8, 9, 10, 11, 12, 13, 14, 15, 16, 17, 18, 19, 20 or greater than 20 amino acids. In some embodiments, the peptide linker is between 1 to 5, 1 to 10, 1 to 15, 1 to 20, 5 to 10, 5 to 15 or 5 to 20 amino acids in length. In a preferred embodiment, the linker is 6 amino acids or less. Exemplary linkers include linear peptides having at least two amino acid residues such as Gly-Gly, Gly-Ala-Gly, Gly-Pro-Ala, Gly-Gly-Gly-Gly-Ser (SEQ ID NO: 1). Suitable linear peptides include poly glycine, polyserine, polyproline, polyalanine and oligopeptides consisting of alanyl and/or serinyl and/or prolinyl and/or glycyl amino acid residues. In some embodiments, the peptide linker has the amino acid sequence selected from the group consisting of Gly9 (SEQ ID NO: 2), Glue (SEQ ID NO: 3), Ser, (SEQ ID NO: 4), Glys-Cys-Pro-Cys (SEQ ID NO: 5), (Gly4-Ser); (SEQ ID NO: 6). Ser-Cys-Val-Pro-Leu-Met-Arg-Cys-Gly-Gly-Cys-Cys-Asn (SEQ ID NO: 7). Pro-Ser-Cys-Val-Pro-Leu-Met-Arg-Cys-Gly-Gly-Cys-Cys-Asn (SEQ ID NO: 8), Gly-Asp-Leu-Ile-Tyr-Arg-Asn-Gln-Lys (SEQ ID NO: 9), and Gly9-Pro-Ser-Cys-Val-Pro-Leu-Met-Arg-Cys-Gly-Gly-Cys-Cys-Asn (SEQ ID NO: 10). In a preferred embodiment, the linker is a Gly-Thr-Gly-Ala (SEQ ID NO: 11) linker.

The domains of stem loop binding protein and the RNA editing proteins of the present disclosure may be in the same or different locations relative to each other. In some embodiments, the stem loop binding domain is intracellular and the dimerization domain of the stem loop binding domain is intracellular. In some embodiments, the stem loop binding domain is intracellular and the dimerization domain of the stem loop binding domain is extracellular. In some embodiments, the stem loop binding domain is extracellular and the dimerization domain of the stem loop binding domain is extracellular. In some embodiments, the RNA editing domain is intracellular and the dimerization domain of the RNA editing protein is intracellular. In some embodiments, the RNA editing domain is intracellular and the dimerization domain of the RNA editing protein is extracellular. In some embodiments, the RNA editing domain is extracellular and the dimerization domain of the RNA editing protein is extracellular. In some embodiments, both the stem loop binding protein and the RNA editing protein are extracellular. In some embodiments, both the stem loop binding protein and the RNA editing protein are intracellular. In some embodiments, the stem loop binding protein is intracellular, the dimerization domain of the RNA editing protein is extracellular and the RNA editing domain is intracellular. In some embodiments, the RNA editing protein is intracellular, the dimerization domain of the stem loop binding protein is extracellular and the stem loop biding domain is intracellular. In embodiments where either the entire protein or the domain of either the stem loop binding protein or the RNA editing protein are extracellular, they may be outside of the cell or exposed to the outside of the cell (i.e., has a transmembrane domain).

The output RNA of the present disclosure may be in any form deemed useful. For instance, the output RNA may be linear or may be circularized. Methods of circularizing RNAs are well known in the art, for example, as described in Chen et al. (Nat. Biotechnol. 2022 Jul. 18. doi: 10.1038/s41587-022-01393-0) which is specifically incorporated by reference herein.

In some embodiments, the output RNA is contained within a delivery vehicle. In some embodiments, the stem loop binding protein is encoded by nucleic acid contained with the delivery vehicle. In some embodiments, the RNA editing protein is encoded by a nucleic acid contained within the delivery vehicle. In some embodiments, the output RNA, the stem loop binding protein and the RNA editing protein are encoded by the same nucleic acid. The delivery vehicle may be any delivery vehicle deemed useful. Delivery vehicles that find use in the present disclosure included, without limitation, a lipid nanoparticle, a recombinant vector, etc. Lipid nanoparticles have been described in the art such as Hou et al. (Nat Rev Mater. 2021; 6 (12): 1078-1094) which is specifically incorporated by reference herein. When the delivery vehicle is a recombinant vector, the recombinant vector includes, without limitation, a plasmid, a viral vector, a cosmid an artificial chromosome, etc. When the delivery vehicle is a recombinant vector, the output RNA, the nucleic acid encoding the stem loop binding protein, the nucleic acid encoding the RNA editing protein or a combination thereof may have natural or synthetic introns. When the delivery vehicle is a recombinant vector, the output RNA, the nucleic acid encoding the stem loop binding protein, the nucleic acid encoding the RNA editing protein or a combination thereof are operably linked to a promoter. Suitable promoters of the present disclosure include, without limitation, a SFFV promoter, a hEF1a, a CMV promoter or a variant thereof, an inducible promoter, a human synapsin promoter (hSyn), a CMV-tetO promoter, a tissue or cell specific promoter, etc.

Depending on the nature of the cell and/or expression construct, protocols of interest may include electroporation, particle gun technology, calcium phosphate precipitation, direct microinjection, viral infection and the like. The choice of method is generally dependent on the type of cell being transformed in the cell associated system and the circumstances under which the transformation is taking place (i.e., in vitro, ex vivo, or in vivo). A general discussion of these methods can be found in Ausubel, et al, Short Protocols in Molecular Biology. 3rd ed., Wiley & Sons, 1995. In some embodiments, lipofectamine and calcium mediated gene transfer technologies are used. After the subject nucleic acids have been introduced into a cell, the cell is may be incubated, normally at 37° C., sometimes under selection, for a period of about 1-24 hours in order to allow for the expression of the output RNA. In mammalian target cells, a number of viral-based expression systems may be utilized to express the output RNA(s). In cases where an adenovirus is used as an expression vector, the output RNA sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

### Methods for Treating a Disease or Condition

Methods for ligand induced ADAR (adenosine deaminase acting on RNA) mediated expression of an RNA coding sequence may also be used to treat an individual for a disease or a condition. In the methods disclosed herein, the RNA coding sequence for expression in the cell (i.e., the cell of the cell associated LIDAR mediated expression system) may promote the survival of the cell or may promote the death of the cell. For instance, if the disease or condition is associated with a cell that is infected by a pathogen or a cancer cell then it may be desirable for the promotion of the death of such cells. In embodiments in which the death of the cell is desired, output protein encoded by the sensor RNA may be any output protein that promotes the death of the cell. Output proteins that promote the death of the cell include, without limitation, a toxin, tumor necrosis factor alpha (TNFa), Fas ligand (FasL), a caspase such as caspase 1, caspase 2, caspase 3, caspase 4, caspase 5, caspase 6, caspase 7, caspase 8, caspase 9, caspase 10, caspase 11, caspase 12, caspase 13 or a variant thereof, etc.

In addition, if the disease or condition is associated with a cell that is infected by a pathogen or a cancer cell it may also be desirable to activate an immune cell to target the infected cell or cancer cell. Immune cells generally include white blood cells (leukocytes) which are derived from hematopoietic stem cells (HSC) produced in the bone marrow. Immune cells also include, e.g., lymphocytes (T cells, B cells, natural killer (NK) cells) and myeloid-derived cells (neutrophil, eosinophil, basophil, monocyte, macrophage, dendritic cells). T cells include all types of immune cells expressing CD3 including T-helper cells (CD4+ cells), cytotoxic T-cells (CD8+ cells). T-regulatory cells (Treg) and gamma-delta T cells. Cytotoxic cells include CD8+ T cells, natural-killer (NK) cells, and neutrophils, which cells are capable of mediating cytotoxicity responses.

In embodiments in which the activation of immune cell is desired, the target RNA that the sensor RNA is directed to may be a target RNA that is specifically expressed in an immune cell. In embodiments wherein the ADAR responsive site does not contain a sensor nucleotide sequence, the cell or cells near the cell express and/or secrete the ligand. In embodiments in which the activation of immune cell is desired, the output RNA may contain a sequence that encodes an output protein that activates or modulates the activity of the immune cell. Non-limiting examples of output proteins that activate immune cells include a chimeric antigen receptor, such as those described above, or a cytokine such as IL-1-like, IL-1α, IL-1β, IL-1RA, IL-18, CD132, IL-2, IL-4, IL-7, IL-9, IL-13, CD1243, 132, IL-15, CD131, IL-3, IL-5, GM-CSF, IL-6-like, IL-6, IL-11, G-CSF, IL-12, LIF, OSM, IL-10-like, IL-10, IL-20, IL-14, IL-16, IL-17, IFN-α, IFN-β, IFN-γ, CD154, LT-β·TNF-α, TNF-β, 4-1BBL, APRIL, CD70, CD153, CD178, GITRL, LIGHT, OX40L, TALL-1, TRAIL, TWEAK, TRANCE, TGF-β1, TGF-2, TGF-β3, Epo, Tpo, Flt-3L, SCF, M-CSF, MSP, etc.

If the disease or condition is associated with the expression of a non-functional protein, a reduced functioning protein or a protein that has an aberrant activity in a disease state relative to a non-disease state then it may be desirable to have an output RNA that is expressed in the diseased cells where, upon contact with the diseased cell that contains the output RNA, the cell produces the output protein where the output protein is a fully functional form of the protein that is non-functioning, has reduced functionality or has aberrant functions.

If the disease or condition is associated with the degradation of a tissue it may be desirable to promote the growth or regrowth of said tissue. In embodiments in which the disease or condition is associated with tissue degradation it may be desirable to have an output RNA that is expressed in the diseased cells where, upon contact with the diseased cell that contains the output RNA, the cell produces the output protein that promotes the growth or regrowth of the tissue. Non-limiting examples of output proteins that promote the growth or regrowth of the tissue include hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angioproteinetins, angiostatin, granulocyte colony stimulating factor (GCSF), erythroproteinetin (EPO), connective tissue growth factor (CTGF), basic fibroblast growth factor (bFGF), acidic fibroblast growth factor (aFGF), epidermal growth factor (EGF), transforming growth factor .alpha. (TGFa), platelet-derived growth factor (PDGF), insulin growth factors I and II (IGF-1 and IGF-11), any one of the transforming growth factor 13-superfamily, including TGFI3, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Given the diversity of cellular activities that may be modulated through the use of the subject cell-associated LIDAR mediated expression system, the instant methods of treatment may be utilized for a variety of applications. As non-limiting examples, the instant methods may find use in a treatment directed to a variety of diseases including but not limited to e.g., Acanthamoeba infection, Acinetobacter infection, Adenovirus infection, ADHD (Attention Deficit/Hyperactivity Disorder), AIDS (Acquired Immune Deficiency Syndrome), ALS (Amyotrophic Lateral Sclerosis), Alzheimer's Disease, Amebiasis, Intestinal (Entamoeba histolytica infection), Anaplasmosis, Human, Anemia, Angiostrongylus Infection, Animal-Related Diseases, Anisakis Infection (Anisakiasis), Anthrax, Aortic Aneurysm, Aortic Dissection, Arenavirus Infection, Arthritis (e.g., Childhood Arthritis, Fibromyalgia, Gout, Lupus (SLE) (Systemic lupus erythematosus), Osteoarthritis, Rheumatoid Arthritis, etc.), Ascaris Infection (Ascariasis), Aspergillus Infection (Aspergillosis), Asthma, Attention Deficit/Hyperactivity Disorder, Autism, Avian Influenza, B virus Infection (Herpes B virus), B. cepacia infection (Burkholderia cepacia Infection), Babesiosis (Babesia Infection), Bacterial Meningitis, Bacterial Vaginosis (BV), Balamuthia infection (Balamuthia mandrillaris infection), Balamuthia mandrillaris infection, Balantidiasis, Balantidium Infection (Balantidiasis), Baylisascaris Infection, Bilharzia, Birth Defects, Black Lung (Coal Workers' Pneumoconioses), Blastocystis hominis Infection, Blastocystis Infection, Blastomycosis, Bleeding Disorders, Blood Disorders, Body Lice (Pediculus humanus corporis), Borrelia burgdorferi Infection, Botulism (Clostridium botulinim), Bovine Spongiform Encephalopathy (BSE), Brainerd Diarrhea, Breast Cancer, Bronchiolitis, Bronchitis, Brucella Infection (Brucellosis), Brucellosis, Burkholderia cepacia Infection (B. cepacia infection), Burkholderia mallei, Burkholderia pseudomallei Infection, Campylobacter Infection (Campylobacteriosis), Campylobacteriosis, Cancer (e.g., Colorectal (Colon) Cancer, Gynecologic Cancers, Lung Cancer, Prostate Cancer, Skin Cancer, etc.), Candida Infection (Candidiasis), Candidiasis, Canine Flu, Capillaria Infection (Capillariasis), Capillariasis. Carbapenem resistant Klebsiella pneumonia (CRKP), Cat Flea Tapeworm, Cercarial Dermatitis, Cerebral Palsy, Cervical Cancer, Chagas Disease (Trypanosoma cruzi Infection), Chickenpox (Varicella Disease), Chikungunya Fever (CHIKV), Childhood Arthritis, German Measles (Rubella Virus), Measles, Mumps, Rotavirus Infection, Chlamydia (Chlamydia trachomatis Disease), Chlamydia pneumoniae Infection, Chlamydia trachomatis Disease, Cholera (Vibrio cholerae Infection), Chronic Fatigue Syndrome (CFS), Chronic Obstructive Pulmonary Disease (COPD), Ciguatera Fish Poisoning, Ciguatoxin, Classic Creutzfeldt-Jakob Disease, Clonorchiasis, Clonorchis Infection (Clonorchiasis), Clostridium botulinim, Clostridium difficile Infection, Clostridium perfringens infection. Clostridium tetani Infection, Clotting Disorders, CMV (Cytomegalovirus Infection), Coal Workers' Pneumoconioses, Coccidioidomycosis, Colorectal (Colon) Cancer, Common Cold, Conjunctivitis, Cooleys Anemia, COPD (Chronic Obstructive Pulmonary Disease), Corynebacterium diphtheriae Infection, Coxiella burnetii Infection, Creutzfeldt-Jakob Disease, CRKP (Carbapenem resistant Klebsiella pneumonia), Crohn's Disease, Cryptococcosis, Cryptosporidiosis, Cryptosporidium Infection (Cryptosporidiosis), Cyclospora Infection (Cyclosporiasis), Cyclosporiasis, Cysticercosis, Cystoisospora Infection (Cystoisosporaiasis), Cystoisosporaiasis, Cytomegalovirus Infection (CMV), Dengue Fever (DF), Dengue Hemorrhagic Fever (DHF), Dermatophytes. Dermopathy, Diabetes, Diamond Blackfan Anemia (DBA), Dientamoeba fragilis Infection, Diphtheria (Corynebacterium diphtheriae Infection), Diphyllobothriasis, Diphyllobothrium Infection (Diphyllobothriasis), Dipylidium Infection, Dog Flea Tapeworm, Down Syndrome (Trisomy 21), Dracunculiasis, Dwarf Tapeworm (Hymenolepis Infection), E. coli Infection (Escherichia coli Infection), Ear Infection (Otitis Media), Eastern Equine Encephalitis (EEE), Ebola Hemorrhagic Fever, Echinococcosis, Ehrlichiosis, Elephantiasis, Encephalitis (Mosquito-Borne and Tick-Borne), Entamoeba histolytica infection, Enterobius vermicularis Infection, Enterovirus Infections (Non-Polio), Epidemic Typhus, Epilepsy, Epstein-Barr Virus Infection (EBV Infection), Escherichia coli Infection, Extensively Drug-Resistant TB (XDR TB). Fasciola Infection (Fascioliasis), Fasciolopsis Infection (Fasciolopsiasis), Fibromyalgia, Fifth Disease (Parvovirus B19 Infection), Flavorings-Related Lung Disease, Folliculitis, Food-Related Diseases, Clostridium perfringens infection, Fragile X Syndrome, Francisella tularensis Infection, Genital Candidiasis (Vulvovaginal Candidiasis (VVC)), Genital Herpes (Herpes Simplex Virus Infection), Genital Warts, German Measles (Rubella Virus), Giardia Infection (Giardiasis), Glanders (Burkholderia mallei), Gnathostoma Infection, Gnathostomiasis (Gnathostoma Infection), Gonorrhea (Neisseria gonorrhoeae Infection), Gout, Granulomatous amebic encephalitis (GAE), Group A Strep Infection (GAS) (Group A Streptococcal Infection), Group B Strep Infection (GBS) (Group B Streptococcal Infection), Guinea Worm Disease (Dracunculiasis), Gynecologic Cancers (e.g., Cervical Cancer, Ovarian Cancer, Uterine Cancer, Vaginal and Vulvar Cancers, etc.), H1N1 Flu. Haemophilus influenzae Infection (Hib Infection), Hand, Foot, and Mouth Disease (HFMD), Hansen's Disease, Hantavirus Pulmonary Syndrome (HPS), Head Lice (Pediculus humanus capitis), Heart Disease (Cardiovascular Health), Heat Stress, Hemochromatosis. Hemophilia, Hendra Virus Infection, Herpes B virus, Herpes Simplex Virus Infection, Heterophyes Infection (Heterophyiasis), Hib Infection (Haemophilus influenzae Infection), High Blood Pressure. Histoplasma capsulatum Disease, Histoplasmosis (Histoplasma capsulatum Disease), Hot Tub Rash (Pseudomonas dermatitis Infection), HPV Infection (Human Papillomavirus Infection), Human Ehrlichiosis, Human Immunodeficiency Virus, Human Papillomavirus Infection (HPV Infection), Hymenolepis Infection, Hypertension, Hyperthermia, Hypothermia, Impetigo, Infectious Mononucleosis, Inflammatory Bowel Discase (IBD), Influenza, Avian Influenza, H1N1 Flu, Pandemic Flu, Seasonal Flu, Swine Influenza. Invasive Candidiasis, Iron Overload (Hemochromatosis), Isospora Infection (Isosporiasis), Japanese Encephalitis, Jaundice, K. pneumoniae (Klebsiella pneumoniae), Kala-Azar, Kawasaki Syndrome (KS), Kernicterus, Klebsiella pneumoniae (K. pneumoniae), La Crosse Encephalitis (LAC), La Crosse Encephalitis virus (LACV), Lassa Fever, Latex Allergies, Lead Poisoning, Legionnaires' Disease (Legionellosis), Leishmania Infection (Leishmaniasis), Leprosy, Leptospira Infection (Leptospirosis), Leptospirosis, Leukemia, Lice, Listeria Infection (Listeriosis), Listeriosis, Liver Disease and Hepatitis, Loa Infection, Lockjaw, Lou Gehrig's Disease, Lung Cancer, Lupus (SLE) (Systemic lupus erythematosus), Lyme Disease (Borrelia burgdorferi Infection), Lymphatic Filariasis, Lymphedema, Lymphocytic Choriomeningitis (LCMV), Lymphogranuloma venereum Infection (LGV), Malaria, Marburg Hemorrhagic Fever, Measles, Melioidosis (Burkholderia pseudomallei Infection), Meningitis (Meningococcal Disease), Meningococcal Disease. Methicillin Resistant Staphylococcus aureus (MRSA), Micronutrient Malnutrition, Microsporidia Infection, Molluscum Contagiosum, Monkey B virus, Monkeypox, Morgellons. Mosquito-Borne Diseases. Mucormycosis, Multidrug-Resistant TB (MDR TB), Mumps, Mycobacterium abscessus Infection. Mycobacterium avium Complex (MAC), Mycoplasma pneumoniae Infection, Myiasis, Naegleria Infection (Primary Amebic Meningoencephalitis (PAM)), Necrotizing Fasciitis, Neglected Tropical Diseases (NTD), Neisseria gonorrhoeae Infection, Neurocysticercosis, New Variant Creutzfeldt-Jakob Disease, Newborn Jaundice (Kernicterus), Nipah Virus Encephalitis, Nocardiosis, Non-Polio Enterovirus Infections, Nonpathogenic (Harmless) Intestinal Protozoa, Norovirus Infection, Norwalk-like Viruses (NLV), Novel H1N1 Flu, Onchocerciasis, Opisthorchis Infection, Oral Cancer, Orf Virus, Oropharyngeal Candidiasis (OPC), Osteoarthritis (OA), Osteoporosis, Otitis Media, Ovarian Cancer, Pandemic Flu, Paragonimiasis. Paragonimus Infection (Paragonimiasis), Parasitic Diseases, Parvovirus B19 Infection. Pediculus humanus capitis, Pediculus humanus corporis, Pelvic Inflammatory Disease (PID), Peripheral Arterial Disease (PAD), Pertussis, Phthiriasis, Pink Eye (Conjunctivitis), Pinworm Infection (Enterobius vermicularis Infection), Plague (Yersinia pestis Infection), Pneumocystis jirovecii Pneumonia, Pneumonia, Polio Infection (Poliomyelitis Infection), Pontiac Fever, Prion Diseases (Transmissible spongiform encephalopathies (TSEs)), Prostate Cancer, Pseudomonas dermatitis Infection, Psittacosis, Pubic Lice (Phthiriasis), Pulmonary Hypertension, Q Fever (Coxiella burnetii Infection), Rabies, Raccoon Roundworm Infection (Baylisascaris Infection), Rat-Bite Fever (RBF) (Streptobacillus moniliformis Infection), Recreational Water Illness (RWI), Relapsing Fever, Respiratory Syncytial Virus Infection (RSV), Rheumatoid Arthritis (RA), Rickettsia rickettsii Infection, Rift Valley Fever (RVF), Ringworm (Dermatophytes), Ringworm in Animals, River Blindness (Onchocerciasis), Rocky Mountain Spotted Fever (RMSF) (Rickettsia rickettsii Infection), Rotavirus Infection, RVF (Rift Valley Fever), RWI (Recreational Water Illness), Salmonella Infection (Salmonellosis), Scabies, Scarlet Fever, Schistosomiasis (Schistosoma Infection), Seasonal Flu, Severe Acute Respiratory Syndrome, Sexually Transmitted Discases (STDs) (e.g., Bacterial Vaginosis (BV), Chlamydia, Genital Herpes, Gonorrhea, Human Papillomavirus Infection, Pelvic Inflammatory Disease, Syphilis, Trichomoniasis, HIV/AIDS, etc.), Shigella Infection (Shigellosis), Shingles (Varicella Zoster Virus (VZV)), Sickle Cell Disease, Single Gene Disorders, Sinus Infection (Sinusitus), Skin Cancer, Sleeping Sickness (African Trypanosomiasis), Smallpox (Variola Major and Variola Minor), Sore Mouth Infection (Orf Virus), Southern Tick-Associated Rash Illness (STARI), Spina Bifida (Myelomeningocele), Sporotrichosis, Spotted Fever Group Rickettsia (SFGR), St. Louis Encephalitis, Staphylococcus aureus Infection, Streptobacillus moniliformis Infection, Streptococcal Diseases, Streptococcus pneumoniae Infection, Stroke, Strongyloides Infection (Strongyloidiasis), Sudden Infant Death Syndrome (SIDS), Swimmer's Itch (Cercarial Dermatitis), Swine Influenza, Syphilis (Treponema pallidum Infection), Systemic lupus erythematosus, Tapeworm Infection (Taenia Infection), Testicular Cancer, Tetanus Disease (Clostridium tetani Infection), Thrush (Oropharyngeal Candidiasis (OPC)), Tick-borne Relapsing Fever, Tickborne Diseases (e.g., Anaplasmosis, Babesiosis, Ehrlichiosis, Lyme Disease, Tourette Syndrome (TS), Toxic Shock Syndrome (TSS), Toxocariasis (Toxocara Infection), Toxoplasmosis (Toxoplasma Infection), Trachoma Infection, Transmissible spongiform encephalopathies (TSEs), Traumatic Brain Injury (TBI), Trichinellosis (Trichinosis), Trichomoniasis (Trichomonas Infection), Tuberculosis (TB) (Mycobacterium tuberculosis Infection), Tularemia (Francisella tularensis Infection), Typhoid Fever (Salmonella typhi Infection), Uterine Cancer, Vaginal and Vulvar Cancers, Vancomycin-Intermediate/Resistant Staphylococcus aureus Infections (VISA/VRSA), Vancomycin-resistant Enterococci Infection (VRE), Variant Creutzfeldt-Jakob Disease (vCJD), Varicella-Zoster Virus Infection, Variola Major and Variola Minor, Vibrio cholerae Infection. Vibrio parahaemolyticus Infection. Vibrio vulnificus Infection. Viral Gastroenteritis, Viral Hemorrhagic Fevers (VHF), Viral Hepatitis. Viral Meningitis (Aseptic Meningitis), Von Willebrand Disease. Vulvovaginal Candidiasis (VVC), West Nile Virus Infection, Western Equine Encephalitis Infection. Whipworm Infection (Trichuriasis), Whitmore's Disease, Whooping Cough, Xenotropic Murine Leukemia Virus-related Virus Infection, Yellow Fever, Yersinia pestis Infection, Yersiniosis (Yersinia enterocolitica Infection), Zoonotic Hookworm, Zygomycosis, and the like.

In some instances, methods of treatment utilizing one or more cell-associated LIDAR mediated expression systems of the instant disclosure may find use in treating a cancer. Cancers will vary and may include but are not limited to e.g., Acute Lymphoblastic Leukemia (ALL), Acute Myeloid Leukemia (AML), Adrenocortical Carcinoma, AIDS-Related Cancers (e.g., Kaposi Sarcoma. Lymphoma, etc.), Anal Cancer. Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers. Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian. Testicular, etc.), Gestational Trophoblastic Disease, Glioma, Hairy Cell Leukemia, Head and Neck Cancer. Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML). Hairy Cell, etc.), Lip and Oral Cavity Cancer, Liver Cancer (Primary), Lobular Carcinoma In Situ (LCIS). Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstrom, etc.), Male Breast Cancer, Malignant Fibrous Histiocytoma of Bone and Osteosarcoma. Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes. Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma. Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer. Uterine Cancer (e.g., Endometrial, etc.), Uterine Sarcoma, Vaginal Cancer, Vulvar Cancer, Waldenström Macroglobulinemia, Wilms Tumor, and the like.

### Compositions

Also provided are compositions for practicing the methods are described in the present disclosure. In general, subject compositions may have a LIDAR mediated expression system as described above. The LIDAR mediated expression system may be contained in a delivery vehicle. In some embodiments the delivery vehicle is a lipid nanoparticle or a recombinant vector as described above.

The LIDAR mediated expression system has the output RNA, the stem loop binding protein, and the RNA editing protein as described above. In some embodiments, the stem loop binding protein is in protein form. In some embodiments, the stem looping binding protein is in the form of a nucleic acid encoding the stem loop binding protein. In some embodiments, the RNA editing protein is in protein form. In some embodiments, the RNA editing protein is in the form of a nucleic acid encoding the RNA editing protein. In some embodiments, the output RNA and the nucleic acid sequence encoding the stem loop binding protein are contained on the same nucleic acid. In some embodiments, the output RNA and the nucleic acid sequence encoding the RNA editing protein are contained on the same nucleic acid. In some embodiments, the nucleic acid sequence encoding the stem loop binding protein, and the nucleic acid sequence encoding the RNA editing protein are contained on the same nucleic acid. In some embodiments, the output RNA, the nucleic acid sequence encoding the stem loop binding protein, and the nucleic acid sequence encoding the RNA editing protein are contained on the same nucleic acid.

In some embodiments, the composition has cells engineered to produce the LIDAR mediated expression system of the present disclosure. In these embodiments, the engineered cells produce the output RNA, the stem loop binding protein, the RNA editing protein or a combination thereof. In some embodiments, the engineered cells produce the protein form of the stem loop binding protein and the RNA editing protein. In some embodiments, the engineered cells produce a nucleic acid encoding the stem loop binding protein, a nucleic acid encoding the RNA editing protein, or a combination thereof. In some embodiments, the engineered cells are engineered using a recombinant vector encoding the output RNA, the stem loop binding protein, the stem loop binding protein, or a combination thereof. In embodiments where the engineered cells are engineered using a recombinant vector, the recombinant vector includes, without limitation, a plasmid, a viral vector, a cosmid an artificial chromosome, etc. When the engineered cells are engineered using a recombinant vector, the output RNA, the nucleic acid encoding the stem loop binding protein, the nucleic acid encoding the RNA editing protein or a combination thereof are operably linked to a promoter. Suitable promoters of the present disclosure include, without limitation, a SFFV promoter, a hEF1a, a CMV promoter or a variant thereof, an inducible promoter, a CMV-tetO promoter, an hSyn promoter, a tissue or cell specific promoter, etc.

The cells may be engineered using any method deemed useful. Depending on the nature of the cell and/or expression construct, protocols of interest may include electroporation, particle gun technology, calcium phosphate precipitation, direct microinjection, viral infection and the like. The choice of method is generally dependent on the type of cell being transformed in the cell associated system and the circumstances under which the transformation is taking place (i.e., in vitro, ex vivo, or in vivo). In some embodiments, lipofectamine and calcium mediated gene transfer technologies are used. After the subject nucleic acids have been introduced into a cell, the cell is may be incubated, normally at 37° C., sometimes under selection, for a period of about 1-24 hours in order to allow for the expression of the output RNA. In mammalian target cells, a number of viral-based expression systems may be utilized to express the output RNA(s). In cases where an adenovirus is used as an expression vector, the output RNA sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. The efficiency of expression may be enhanced by the inclusion of appropriate transcription enhancer elements, transcription terminators, etc.

In some embodiments, the composition is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the composition may further include a preservative. Suitable preservatives include, but are not limited to, a benzyl alcohol, phenol, chlorobutanol, benzalkonium chloride, and the like. In many cases, the formulation is stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

The subject composition can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The composition may be administered in a unit dosage form and may be prepared by any methods well known in the art. Such methods include combining the LIDAR mediated expression system with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

### Kits

Also provided are kits for practicing the methods described in the present disclosure. In general, subject kits may contain the LIDAR mediated expression system or components thereof, e.g., as described above. The LIDAR mediated expression system as described above (or components thereof) may be contained in a delivery vehicle. The delivery vehicle may be any delivery vehicle deemed useful. Delivery vehicles that find use in the present disclosure include, without limitation, a lipid nanoparticle, a recombinant vector, etc. In some cases, the kit further has a ligand that specifically binds to the stem loop binding protein, the RNA editing protein or a combination thereof. When the kit has a coding sequence of the LIDAR mediated expression system as described above or an individual component thereof it may be in a recombinant vector as described above.

The LIDAR mediated expression system has the output RNA, the stem loop binding protein, and the RNA editing protein as described above. In some embodiments, the stem loop binding protein is in protein form. In some embodiments, the stem looping binding protein is in the form of a nucleic acid encoding the stem loop binding protein. In some embodiments, the RNA editing protein is in protein form. In some embodiments, the RNA editing protein is in the form of a nucleic acid encoding the RNA editing protein. In some embodiments, the output RNA and the nucleic acid sequence encoding the stem loop binding protein are contained on the same nucleic acid. In some embodiments, the output RNA and the nucleic acid sequence encoding the RNA editing protein are contained on the same nucleic acid. In some embodiments, the nucleic acid sequence encoding the stem loop binding protein, and the nucleic acid sequence encoding the RNA editing protein are contained on the same nucleic acid. In some embodiments, the output RNA, the nucleic acid sequence encoding the stem loop binding protein, and the nucleic acid sequence encoding the RNA editing protein are contained on the same nucleic acid.

A subject kit can include any combination of components for performing the methods of the present disclosure. The components of a subject kit can be present as a mixture or can be separate entities. In some cases, components are present as a lyophilized mixture. In some cases, the components are present as a liquid mixture. Components of a subject kit can be in the same or separate containers, in any combination.

A subject kit can include a cell containing the LIDAR-mediated expression system. The cell may be any type of cell deemed useful. Nonlimiting examples of cells that find use in the present disclosure includes, without limitation, a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gynmosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. In some embodiments, the cell is contained within a device. In some embodiments, the device is used for the detection of a protein, a chemical, a lipid, a small molecule, a compound, a nucleic acid, a nucleic acid sequence, an element, etc.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1

The first platform for creating modular post-transcriptional synthetic receptors, dubbed LIDAR (Ligand-Induced Dimerization Acting on RNA editing) was designed (FIG. 1). LIDAR leverages specific adenosine (A)->inosine (I) editing mediated by ADAR (double-stranded RNA-specific adenosine deaminase) (16). LIDAR consists of three components: one dimerizing partner fused to the deaminase domain of ADAR2 (ADAR2DD), the other partner fused to phage MS2 coat protein (MCP) (18), and a reporter RNA molecule harboring an in-frame stop codon in a double-stranded region from a natural substrate for ADAR2 and MS2 hairpins for binding MCP (19). To accommodate as many different ligands as possible, three types of dimerization were co-opted, 1) between two cytosolic domains binding non-overlapping regions on the ligand (FIG. 1A, named ‘cLIDAR’ after cytosolic, 2) between two extracellular domains binding non-overlapping regions on the ligand (FIG. 1B, named ‘eLIDAR’ after extracellular), and 3) the recruitment of a cytosolic domain by an activated receptor (FIG. 1C, named ‘tLIDAR’ after based on Tango). Ligand-induced dimerization brings ADAR2DD closer to its substrate to increase A->I editing. Inosine is treated mostly as guanosine (G) in translation, converting the stop codon to a tryptophan codon and expressing the output polypeptide downstream. “Self-cleaving” 2A peptides (20) were used to insulate the flanking coding sequences from the peptide encoded by the ADAR substrate and the MS2 hairpins. Compared to synNotch, the only modular synthetic receptor demonstrated to synergize with CAR (8-10), LIDAR not only offers the advantage of RNA compatibility, but also broadens the potential ligand pool, because synNotch requires surface-tethered ligands. In contrast, LIDAR can be programmed to respond to both surface-tethered and soluble ligands.

To demonstrate the feasibility of LIDAR, a rapalog responsive cLIDAR (FIG. 1A) was generated. A substantial output induction (86×) was achieved by rapalog induced dimerization (FIG. 1D). A similar rapalog responsive eLIDAR was also generated (FIG. 1B). A substantial output induction (30×) was also achieved by rapalog induced dimerization (FIG. 1E).

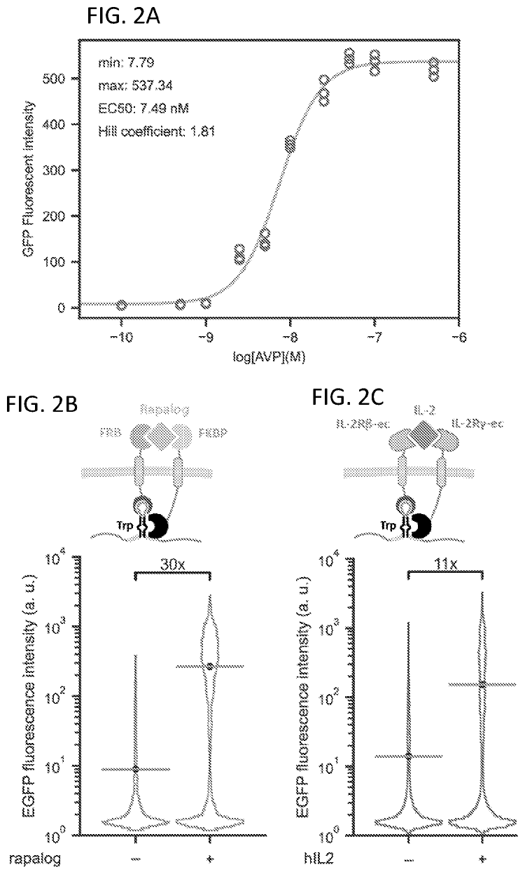

To demonstrate the feasibility of tLIDAR and benchmark it against transcriptional receptors, vasopressin-responsive tLIDAR was first optimized (FIG. 1C, “AVP-tLIDAR”), using the same vasopressin receptor 2 (AVPR2) and beta arrestin (ARRB2) as the original TANGO receptors (11) (transcriptional receptors that rely on proteolytic cleavage induced by GPCR-arrestin interaction). A ˜70-fold output dynamic range was achieved (FIG. 2A), and the input-output relation is comparable to TANGO. A substantial output induction (100×) was achieved upon binding to AVP (FIG. 1F) An eLIDAR for IL-2 using two ectodomains from the IL-2 receptor was then created (FIG. 2C), and optimized it till it increases output in response to IL-2 expressed in the same cells (FIG. 2C).

Unless otherwise noted, the receptor components are encoded on plasmids and transfected into human embryonic kidney (HEK) 293 cells. The DNA plasmids will speed up engineering through their convenient production and delivery, but do not affect the LIDAR mechanism that still operates on the mRNA produced from the plasmids. The outputs are measured using flow cytometry, and quantified after gating on a co-transfection marker to enrich highly transfected cells. This workflow enables a fast six-day design-build-test-learn cycle, which already expedited iterative engineering for our prototypes and will continue to facilitate our engineering efforts.

### Example 2

### Expand the Input/Output Repertoires of LIDAR.

An ideal platform for building receptors should accommodate diverse inputs and outputs. For inputs, tLIDAR's versatility is assessed against the large GPCR family for different ligands. In comparison, the con of cLIDAR and eLIDAR is the need for two distinct ligand-binding domains, whereas the pro is compatibility with ligands for which no GPCRs exist, cLIDAR and eLIDAR designs are tested against several such ligands. For outputs, because of the many use cases, including CAR T cells, CAR natural killer cells, CAR T regulator cells, CAR macrophages, etc.; that require the control of intercellular communication, proteins that are secreted or displayed on the cell surface are focused on.

To assess tLIDAR's versability, ligands with distinct features are targeted: dopamine (neuro-transmitter), morphine (analgesic and addictive substance), CCL20 (chemokine), and thrombin (critical for blood coagulation; besides, it activates GPCR through cleavage rather than binding). TANGO receptors (22-24) for these ligands enable direct benchmarking of LIDAR performance. The AVP-tLIDAR topology is followed (FIG. 1C), except for using the corresponding GPCRs and arrestins. As an option, the V2 tail from AVPR2 is attached to the C termini of these receptors, known to enhance ARRB2 recruitment by active GPCR (22). The input-output function of each receptor is measured in response to the respective externally administered ligand.

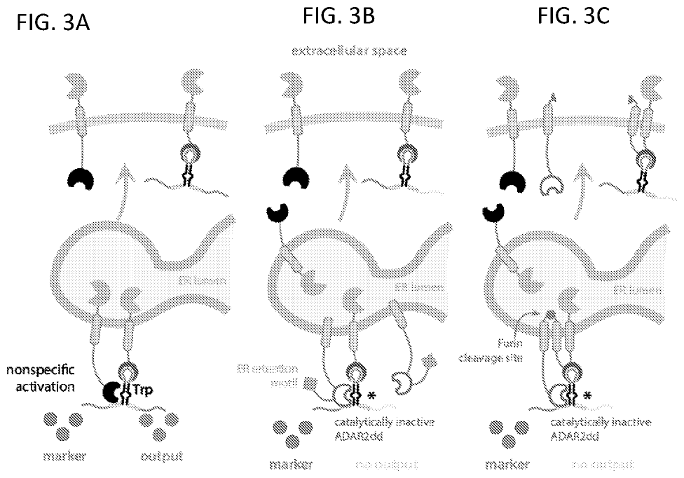

For compatibility with more diverse ligands, cLIDAR and eLIDAR are optimized. Although IL-2-eLIDAR responds to cell-autonomously expressed IL-2 (FIG. 2C). Because ADAR2DD retains residual affinity to dsRNA, increased effective concentration might lead to baseline editing as LIDAR components co-transit through the endoplasmic reticulum (ER) and Golgi apparatus (FIG. 3A). Cell-autonomously expressed IL-2 would dimerize eLIDAR throughout ER and Golgi, at no disadvantage compared to the baseline, whereas externally administered IL-2 only dimerizes eLIDAR at the cell surface, and the input-induced signal is overwhelmed by the baseline generated from the larger membrane areas in ER and Golgi. This does not present an issue for tLIDAR, because arrestin and the reporter RNA are cytosolic before receptor activation. Therefore, the key for eLIDAR is to reduce the baseline in ER and Golgi without affecting ligand-induced output at the cell membrane. A membrane-tethered, catalytically inactive ADAR2DD (ADAR2DDmut) is overexpressed, and used as a retention signal (25) to enrich it in ER and Golgi (FIG. 3B). Expressed at a level higher than ADAR2DD, it competitively inhibits the editing of the reporter RNA during secretion until eLIDAR transits to the cell membrane. Alternatively, the MCP half of eLIDAR is fused to a membrane-tethered, cytosol-facing ADAR2DDmut through a luminal linker cleavable by the Furin protease specifically present in trans Golgi (26) (FIG. 3C). In ER and cis Golgi, ADAR2DDmut is tethered closely to the reporter RNA and outcompete ADAR2DD, until this advantage is negated after eLIDAR encounters Furin. Linkers are screened and variants of ADAR2DDmut for both strategies. Once successful, eLIDAR against additional ligands is assessed, such as VEGF (key mediator of cancer angiogenesis) and TNF (critical roles in cancer and autoimmune diseases). Ligands are selected based on not only their potential as disease biomarkers, but also the availability of dual binding domains, either as ectodomains from their endogenous receptors (27) or as single-domain antibodies (12).

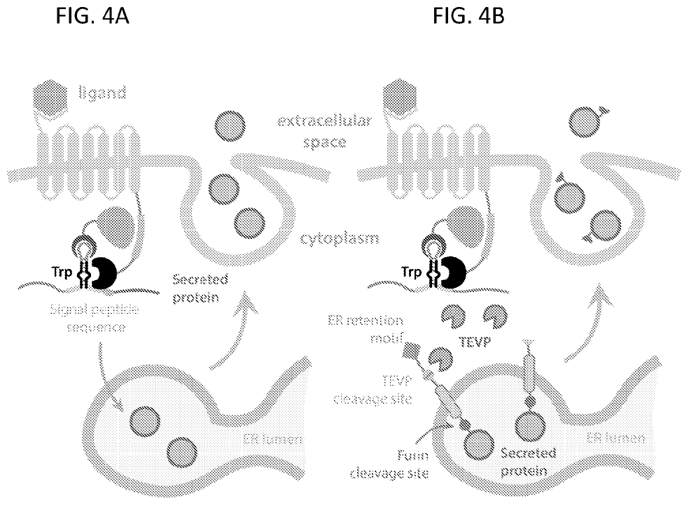

To couple LIDAR outputs to intercellular communications, secreted embryonic alkaline phosphatase (SEAP) is started with, quantified using a colorimetric reaction performed on the cell culture supernatant (28) to expedite engineering. SEAP is first appended after the last 2A peptide in the AVP-LIDAR output (FIG. 4A), because signal peptides have been shown to function properly in the post-2A position (20). Signal peptides are screened to maximize AVP-induced SEAP output. However, unlike typical cytosolic mRNA used to express 2A peptides, the RNA reporter in LIDAR is enriched near the cell membrane. When the nascent signal peptide has difficulty diffusing to ER for protein secretion. “RELEASE” (29) technology is used to convert a cytosolic LIDAR output to the secretion of arbitrary proteins. A specific viral protease from LIDAR is outputted, which removes the ER-retention signal on a membrane protein and enable the latter to be exported to Golgi and beyond (FIG. 4B). The final output is either tethered to the membrane, or, as in the case of SEAP, cleaved at an intentionally incorporated Furin site in trans Golgi and freed from the membrane. The generalizability is tested by outputting secreted proteins such as IL-12 (an immunostimulatory cytokine, quantified using ELISA) and cell-surface proteins such as CAR (quantified using immunostaining against an epitope tag and then flow cytometry).

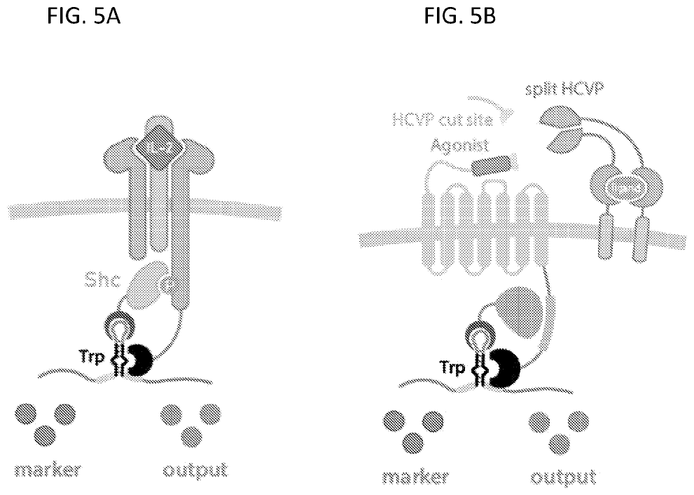

Alternatives: Two alternatives are explored. First, similarly to the GPCR-arrestin interaction, many cytokine and growth factor-activated receptors recruit proteins containing SH2-domains from the cytosol (30), tLIDAR is expanded to such ligands, starting with IL-2 receptor and the correspondingly recruited cytosolic domain (31) (FIG. 5A). Second, for ligands where no natural receptors exist or the use of synthetic binding domains is preferred for other reasons, we can build on Thrombin-tLIDAR is built upon (FIG. 5B). The protease-activated receptors are taken (GPCRs activated by thrombin cleavage) from tLIDAR, and replace the cleavage site for Thrombin with that for an orthogonal viral protease. The protease is split (32), displayed on the cell surface, and fused each half to a binding domain for a distinct epitope on the ligand. The baseline in the secretory pathway is mitigated by fusing the protease halves to catalytically inactive, complementary halves, removable by Furin. IL-2-binding ectodomains are started to prototype this concept.

### Example 3

### Optimize the Output Dynamic Range and Threshold Behavior of LIDAR.

For any LIDAR application, the desirable features include maximal input-induced output and minimal input-independent baseline. Furthermore, although an output that tracks the level of the input is valuable as an observational tool, for cell therapies a switch-like behavior is often necessary, where the output “flips” from OFF to ON as the input level increases over a narrow range. Such a threshold behavior helps quantitatively distinguish cells or microenvironments expressing different levels of markers, more common than all-or-none markers. A set of design rules is required for tuning new receptors to achieve these features.

AVP-tLIDAR is used as a test case to establish preliminary rules, and the rules are tested on the other receptors. Optimal stoichiometry is established between tLIDAR components. The presence of three components makes combinatorial, exhaustive titration impractical. Therefore, experimental titration is guided using computational simulation, tLIDAR behavior is modeled using ordinary differential equations, and titrations are performed in silico for the minimal set of informative titrations every time a new component is tested.

In parallel with titration, tLIDAR components are engineered to improve the output dynamic range. First, despite the 70-fold dynamic range (FIG. 2A), a non-negligible AVP-independent baseline is detected (not shown). The same level of baseline is observed when the RNA reporter is co-expressed with only AVPR2-ADAR2DD but not ARRB2-MCP (not shown), suggesting that the major contributor to baseline is ADAR2DD editing its substrate, probably due to ADAR2DD's residual dsRNA affinity. Mutants or truncations of the ADAR2 substrate are screened for in the RNA reporter that reduce baseline editing. Second, the pilot screen showed that shorter linkers between AVPR2 and ADAR2DD lead to larger output dynamic ranges (not shown). In addition to increasing the effective local concentration of ADAR2DD with respect to the reporter, ARRB2-AVPR2 binding alter the conformation and deaminase activity of the physically coupled ADAR2DD. To screen for other coupling modes that increase deaminase activity upon dimerization, the linker is further shortened and use rigid residues instead of the current glycines and serines.

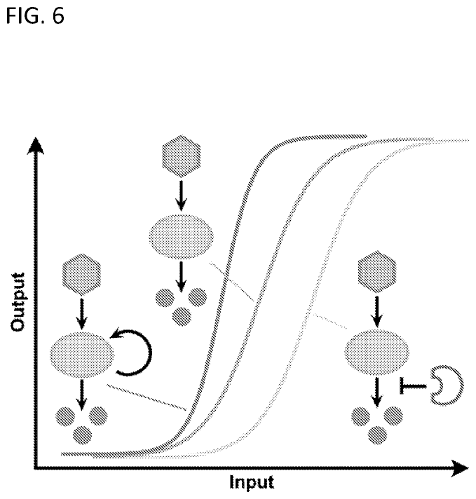

AVP-tLIDAR already exhibits thresholding behavior with a Hill coefficient of 1.8 (FIG. 2A). It is improved in two ways (FIG. 6): enhance ultrasensitivity (i.e., larger Hill coefficient and more switch-like), and engineer tunability (i.e., achieving different threshold values for different inputs). A positive feedback loop is implemented, a motif known to enhance ultrasensitivity (10) and compatible with LIDAR, where LIDAR outputs more of the receptor parts of itself (FIG. 6). The output modules are utilized to express the cell-surface protein AVPR2-ADAR2DD. To tune the threshold, at the protein and the circuit levels are engineered using orthogonal strategies with respect to ultrasensitivity, so that these two features can be tuned independently. At the protein level, AVPR2 variants with different AVP affinities (33) are used; at the circuit level, competitive inhibition is used from a co-expressed ADAR2DDmut (FIG. 6), a motif with known threshold-tuning capabilities (34). The benefit of protein engineering is compactness, as no additional components need to be encoded, while the benefit of circuit engineering is the generalizability to other ligands, for which receptors with different affinities might not exist and require substantial additional engineering efforts. Optimization in this section is performed iteratively between computational simulation and experimental screen, where the former is used to propose candidate circuits and guide optimization for the latter, and the latter to update parametric constraints for the former.

Alternatives: A “poly-transfection” paradigm (35) is adopted, transfecting a single population of cells with multiple independent rounds, and take advantage of the inherent stochasticity of DNA transfection to naturally vary the concentration of each plasmid by several orders of magnitude. From a single transfected well, we will retrieve high-dimensional “titration” data to better constrain the models.

### Example 4

Deliver LIDAR as a Single Transcript Encoded on mRNA.

To demonstrate LIDAR's key advantage, i.e., compatibility with compact mRNA delivery, its optimal performance in such a context is validated.

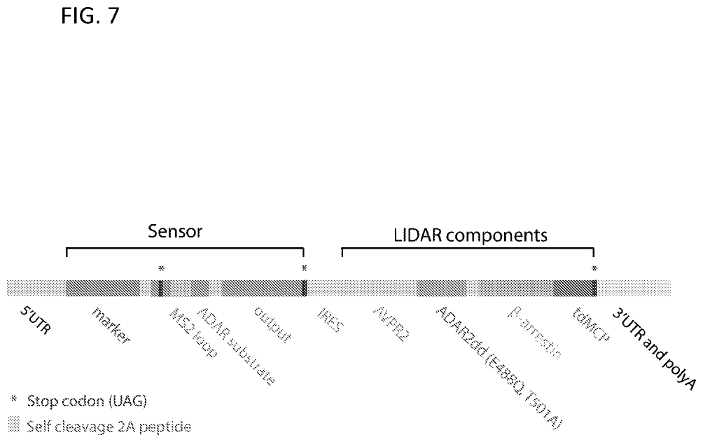

Strategy: AVP-tLIDAR is started with before generalizing to other receptors. To maintain optimal stoichiometry while refactoring LIDAR components to single-transcript delivery, 2A peptides and internal ribosome entry sites (IRES) are used. To use AVP-tLIDAR as an example (FIG. 7): 2A peptides are used to separate components optimized to operate at ˜1:1 ratios; IRES is used for two reasons, to generate free N- or C-terminus to avoid interfering with subcellular localization (e.g., the co-translational membrane insertion of AVPR2-ADAR2DD), and to titrate the relative expression levels of components separated by them. A library of IRES variants (36) is chosen from to match the optimal ratios determined through plasmid titration. The same workflow is applied to at least two more tLIDAR receptors, two eLIDAR receptors, one ultrasensitive receptor, and one with tunable threshold. Fluorescent protein is used as the output to expedite the workflow, but also validate SEAP and CAR outputs for at least two receptors. Each final version is encoded as a single transcript on a DNA plasmid, transfected into HEK293 cells, and validated that its input-output relationship is consistent with previous results utilized in multi-plasmid encoding.

For mRNA delivery, each single transcript-encoded LIDAR receptor from the previous paragraph is introduced using in vitro transcribed mRNA into HEK293 cells using liposomes and lipid nanoparticles (LNP), and flow cytometric measurements are used to benchmark them against those expressed from transfected DNA plasmids. The mRNA is also circularized (37) to extend its half-life and enhance output.

Alternatives: 2A peptides and IRES “lock in” optimal ratios and do not allow for the fine tuning of stoichiometry in different cells/individuals or at different stages after the cells are deployed in vivo. LIDAR components are fused to degrons, and their levels are tuned using small molecules that stabilize the degrons (38). The single transcript will be ˜7 kb long. Although replicon mRNAs up to 9 kb have been delivered in vivo (39) using LNP, when the length poses a manufacturing/delivery challenge, LIDAR components are co-delivered on multiple mRNA molecules.

### Example 5

### Demonstrate LIDAR's Potential to Improve CAR T Cell Therapy.

Ablation of active fibroblasts to treat heart injury is a compelling use case for LIDAR. In situ RNA-mediated creation of CAR T cells has been demonstrated for this purpose (2), but the targeted marker, fibroblast activation protein (FAP), is also present on several other cell types and extracardiac active fibroblasts. Possible adverse effects are mitigated by using LIDAR to express CAR in response to a second marker. As the first steps towards that vision and a proof of principle for LIDAR's utility, LIDAR receptors for relevant markers are developed and LIDAR functionality in T cells is validated.

To distinguish cardiac active fibroblasts from off-target populations, LIDAR receptors for three scenarios are created. First, the fibrogenic microenvironment in heart injury is associated with soluble factors such as CCL2 (40) and CTGF (41), which are leveraged to distinguish such environments from non-fibrogenic ones, tLIDAR are built using the GPCR for CCL2, and eLIDAR using single-chain antibodies/nanobodies against CTGF (42). The natural CTGF dimers bridge the two halves of eLIDAR even if they use the same binding domain. Second, genes with elevated expression in fibroblasts such as CCL19 and IL-6 are helpful to reduce the ablation of non-fibroblast cells, because the few other cell types expressing these two genes do not overlap with other FAP-expressing cells (Human Protein Atlas), tLIDAR are built using the GPCR for CCL19, and either eLIDAR or tLIDAR for IL-6, depending on the IL-6-dependent dimerization between membrane proteins IL-6R and gp130, or that between gp130 and the cytosolic proteins it recruits (43). Third, the most conceptually straightforward way is to target a second marker also elevated in active fibroblasts in heart infarctions, such as THY1 and Postn (3). A single-chain antibody against THY1 has been reported (44). Because there are ELISA assays with antibodies binding non-overlapping epitopes on THY1 and Postn, the possibility of converting these antibodies to single-chain antibodies are explored, which is then used in eLIDAR. A subset of these receptors is used to output FAP-CAR and used in the previous workflow 3 to generate single-transcript receptors, optimized and validated in HEK293 cells.

To use LIDAR receptors in T cells, all optimized single-transcript receptors are validated from the previous paragraph in the immortalized Jurkat cell line. Although Jurkat does not recapitulate all aspects of T cell biology, it provides an engineering chassis to iteratively tweak LIDAR. LIDAR->FAP-CARS is encoded on lentiviral vectors (to facilitate engineering though LIDAR still operates on the mRNAs produced from these vectors), transduce Jurkat cells, externally administer the cognate ligands, and quantify CAR expression using epitope staining. The input-output curves of these LIDAR receptors are obtained, and, for those whose performance deviate substantially from the HEK293 results, the components are titrated to reestablish optimal stoichiometry. To functionally assay LIDAR->CARs, primary human CD8+ T cells are purchased, and transduced with the optimized lentiviral vectors encoding LIDAR->FAP-CAR. The modified T cells are co-cultured with control K562 cells or K562 engineered to express either or both of FAP and the second marker. ELISA is used to measure immunostimulatory cytokines secreted by active T cells (e.g., IL-2) and quantify the killing of K562 cells is quantified to measure T cell-mediated cytotoxicity. For each tested receptor, the extent to which LIDAR-mediated AND logic is functional is characterized, i.e., substantial killing of target cells is observed only when the target cell expresses both FAP and the second marker. The ultrasensitive and threshold tuning strategies used previously are used, and the expression level of these second markers is titrated to validate the quantitative performance of LIDAR receptors. The final test produces mRNAs encoding the validated receptors, delivers them using LNPs into primary T cells, and recapitulates the same level of T cell activation and K562 killing as that observed for lenti-delivered LIDAR.

Alternatives: LIDAR and CAR are switched and FAP is used as the “second marker”. Because FAP is a protease (45), Thrombin-tLIDAR is adopted for FAP by switching the cleavage site. The output CAR may then be engineered to target one of the markers mentioned above, such as THY1.

### REFERENCES

1. Foster, J. B., Barrett, D. M. & Kariko, K. The Emerging Role of In Vitro-Transcribed mRNA in Adoptive T Cell Immunotherapy. Mol. Ther. 27, 747-756 (2019).
2. Rurik, J. G, et al. CAR T cells produced in vivo to treat cardiac injury. Science 375, 91-96 (2022).
3. Aghajanian, H, et al. Targeting cardiac fibrosis with engineered T cells. Nature 573, 430-433 (2019).
4. Amor, C, et al. Senolytic CAR T cells reverse senescence-associated pathologies. Nature 583, 127-132 (2020).
5. Gross, G. & Eshhar, Z. Therapeutic Potential of T Cell Chimeric Antigen Receptors (CARs) in Cancer Treatment: Counteracting Off-Tumor Toxicities for Safe CAR T Cell Therapy. Annu. Rev. Pharmacol. Toxicol. 56, 59-83 (2016).
6. Manhas, J., Edelstein, H. I., Leonard, J. N. & Morsut, L. The evolution of synthetic receptor systems. Nat. Chem. Biol. 18, 244-255 (2022).
7. Morsut, L, et al. Engineering customized cell sensing and response behaviors using synthetic notch receptors. Cell 164, 780-791 (2016).
8. Roybal, K. T, et al. Engineering T Cells with Customized Therapeutic Response Programs Using Synthetic Notch Receptors. Cell 167, 419-432.e16 (2016).
9. Williams, J. Z, et al. Precise T cell recognition programs designed by transcriptionally linking multiple receptors. Science 370, 1099-1104 (2020).
10. Hernandez-Lopez, R. A, et al. T cell circuits that sense antigen density with an ultrasensitive threshold. Science 371, 1166-1171 (2021).
11. Barnea, G, et al. The genetic design of signaling cascades to record receptor activation. Proc. Natl. Acad. Sci. USA 105, 64-69 (2008).
12. Schwarz, K. A., Daringer, N. M., Dolberg, T. B. & Leonard, J. N. Rewiring human cellular input-output using modular extracellular sensors. Nat. Chem. Biol. 13, 202-209 (2017).
13. Daringer, N. M., Dudek, R. M., Schwarz, K. A. & Leonard, J. N. Modular extracellular sensor architecture for engineering mammalian cell-based devices. ACS Synth. Biol. 3, 892-902 (2014).
14. Wang, W., Kim, C. K. & Ting, A. Y. Molecular tools for imaging and recording neuronal activity. Nat. Chem. Biol. 15, 101-110 (2019).
15. Andries, O., Kitada, T., Bodner, K., Sanders, N. N. & Weiss, R. Synthetic biology devices and circuits for RNA-based “smart vaccines”: a propositional review. Expert Rev. Vaccines 14, 313-331 (2015).
16. Nishikura, K. Functions and regulation of RNA editing by ADAR deaminases. Annu. Rev. Biochem. 79, 321-349 (2010).
17. Kaseniit, K. E, et al. Modular and programmable RNA sensing using ADAR editing in living cells. BioRxiv (2022). doi: 10.1101/2022.01.28.478207
18. Katrekar, D, et al. In vivo RNA editing of point mutations via RNA-guided adenosine deaminases. Nat. Methods 16, 239-242 (2019).
19. Rodriques, S. G, et al. RNA timestamps identify the age of single molecules in RNA sequencing. Nat. Biotechnol. 39, 320-325 (2021).
20. Fang, J, et al. Stable antibody expression at therapeutic levels using the 2A peptide. Nat. Biotechnol. 23, 584-590 (2005).
21. Bayle, J. H, et al. Rapamycin analogs with differential binding specificity permit orthogonal control of protein activity. Chem. Biol. 13, 99-107 (2006).
22. Kroeze, W. K, et al. PRESTO-Tango as an open-source resource for interrogation of the druggable human GPCRome. Nat. Struct. Mol. Biol. 22, 362-369 (2015).
23. Cho, K. F, et al. A light-gated transcriptional recorder for detecting cell-cell contacts. Elife 11, (2022).
24. Inagaki, H. K, et al. Visualizing neuromodulation in vivo: TANGO-mapping of dopamine signaling reveals appetite control of sugar sensing. Cell 148, 583-595 (2012).
25. Nilsson, T. & Warren, G. Retention and retrieval in the endoplasmic reticulum and the Golgi apparatus. Curr. Opin. Cell Biol. 6, 517-521 (1994).
26. Molloy, S. S., Anderson, E. D., Jean, F. & Thomas, G. Bi-cycling the furin pathway: from TGN localization to pathogen activation and embryogenesis. Trends Cell Biol. 9, 28-35 (1999).
27. Mukai, Y, et al. Solution of the structure of the TNF-TNFR2 complex. Sci. Signal. 3, ra83 (2010).
28. Scheller, L., Strittmatter, T., Fuchs, D., Bojar, D. & Fussenegger, M. Generalized extracellular molecule sensor platform for programming cellular behavior. Nat. Chem. Biol. 14, 723-729 (2018).
29. Vlahos, A. E, et al. Protease-controlled secretion and display of intercellular signals. Nat. Commun. 13, 912 (2022).
30. Neel, B. G., Gu, H. & Pao, L. The ‘Shp’ing news: SH2 domain-containing tyrosine phosphatases in cell signaling. Trends Biochem. Sci. 28, 284-293 (2003).
31. Migone, T. S, et al. Recruitment of SH2-containing protein tyrosine phosphatase SHP-1 to the interleukin 2 receptor; loss of SHP-1 expression in human T-lymphotropic virus type I-transformed T cells. Proc. Natl. Acad. Sci. USA 95, 3845-3850 (1998).
32. Gao, X. J., Chong, L. S., Kim, M. S. & Elowitz, M. B. Programmable protein circuits in living cells. Science 361, 1252-1258 (2018).
33. Makita, N., Manaka, K., Sato, J. & liri, T. V2 vasopressin receptor mutations. Vitam Horm 113, 79-99 (2020).
34. Ferrell, J. E. Tripping the switch fantastic: how a protein kinase cascade can convert graded inputs into switch-like outputs. Trends Biochem. Sci. 21, 460-466 (1996).
35. Gam, J. J., DiAndreth, B., Jones, R. D., Huh, J. & Weiss, R. A “poly-transfection” method for rapid, one-pot characterization and optimization of genetic systems. Nucleic Acids Res. 47, e106 (2019).
36. Koh, E. Y. C, et al. An internal ribosome entry site (IRES) mutant library for tuning expression level of multiple genes in mammalian cells. PLoS One 8, e82100 (2013).
37. Chen, Y. G, et al. N6-Methyladenosine Modification Controls Circular RNA Immunity. Mol. Cell 76, 96-109.e9 (2019).
38. Rakhit, R., Navarro, R. & Wandless, T. J. Chemical biology strategies for posttranslational control of protein function. Chem. Biol. 21, 1238-1252 (2014).
39. Geall, A. J, et al. Nonviral delivery of self-amplifying RNA vaccines. Proc. Natl. Acad. Sci. USA 109, 14604-14609 (2012).
40. Hanna, A. & Frangogiannis, N. G. Inflammatory cytokines and chemokines as therapeutic targets in heart failure. Cardiovasc Drugs Ther 34, 849-863 (2020).
41. Daniels, A., van Bilsen, M., Goldschmeding, R., van der Vusse, G. J. & van Nieuwenhoven, F. A. Connective tissue growth factor and cardiac fibrosis. Acta Physiol (Oxf) 195, 321-338 (2009).
42. Wang, X, et al. A novel single-chain-Fᵥ antibody against connective tissue growth factor attenuates bleomycin-induced pulmonary fibrosis in mice. Respirology 16, 500-507 (2011).
43. Garbers, C., Aparicio-Siegmund, S. & Rose-John, S. The IL-6/gp130/STAT3 signaling axis: recent advances towards specific inhibition. Curr. Opin. Immunol. 34, 75-82 (2015).
44. Abou-Elkacem, L, et al. Thyl-Targeted Microbubbles for Ultrasound Molecular Imaging of Pancreatic Ductal Adenocarcinoma. Clin. Cancer Res. 24, 1574-1585 (2018).
45. Edosada, C. Y, et al. Peptide substrate profiling defines fibroblast activation protein as an endopeptidase of strict Gly (2)-Pro (1)-cleaving specificity. FEBS Lett. 580, 1581-1586 (2006).
46. Gao, X. J., Chong, L. S., Ince, M. H., Kim, M. S. & Elowitz, M. B. Engineering multiple levels of specificity in an RNA viral vector. BioRxiv (2020). doi: 10.1101/2020.05.27.119909

### Example 6

### Introduction

Recent developments of modular synthetic receptors have enabled cells to detect diverse extracellular ligands and generate customized outputs (1). They have empowered basic research and biomedical applications, such as visualizing neurotransmitter release (2) and increasing the targeting specificity of chimeric antigen receptor (CAR) T cells (3). Meanwhile, the success of mRNA vaccines demonstrates a transformative modality of therapeutic delivery, because its transient and non-mutagenic nature offers superior safety over traditional DNA vectors (4). For example, mRNA-mediated CAR expression in vivo holds the promise to substantially decrease the cost of such therapies. Given that all existing modular synthetic receptors operate through transcriptional regulation, there remains a critical opportunity at the interface of these two thriving fields-RNA-compatible synthetic receptors based on novel post-transcriptional actuation mechanisms.

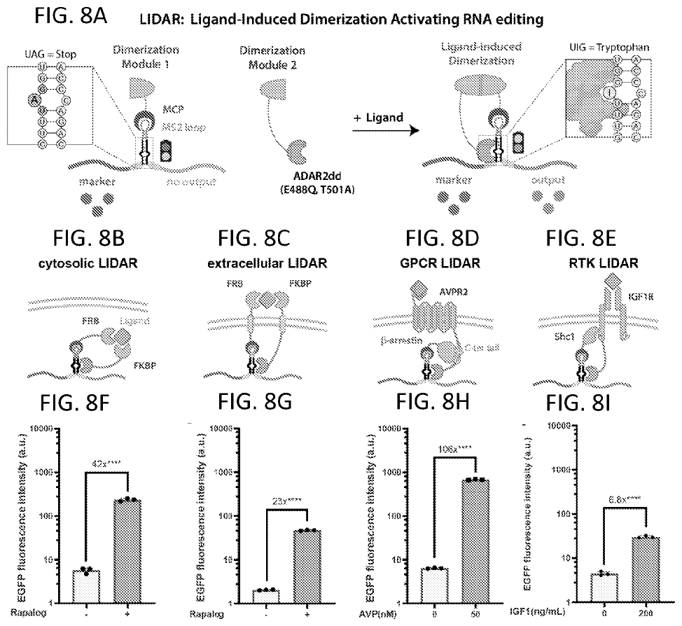

After conceptually and experimentally assessing multiple mechanisms, it was determined that post-transcriptional receptors based on RNA editing are most likely to operate robustly, as they would require standalone enzymatic reactions regardless of the cellular contexts. Specifically, inspired by previous work (5), adenosine deaminases acting on RNA (ADAR) that edits adenosine (A) to inosine (I), treated as guanosine (G) in translation was focused on. Each ADAR consists of a catalytic deaminase domain (e.g., ADAR2dd) and double-strand RNA binding domains (dsRBDs) that recruit it to editing sites. In a design dubbed LIDAR (Ligand-Induced Dimerization Activating RNA editing), the dsRBDs on ADAR2 with domains that recruit ADAR2dd was replaced with a synthetic RNA substrate in the presence of a specific ligand (FIG. 8A). LIDAR consists of three components: two customizable dimerization domains—one fused to an ADAR2dd and the other to a specific RNA binding protein (RBP)—and a reporter RNA molecule harboring multiple RBP recruiting sequences and an in-frame stop codon (UAG) within a double-stranded region as an ADAR substrate. When the reporter RNA is brought into the proximity of ADAR2dd, the stop codon gets edited away, generating a permissive tryptophan codon and the translation of the downstream payload.

Here it was demonstrated that LIDAR is compatible with a variety of receptor architectures and is highly modular, capable of sensing diverse ligands and actuating various functional outputs. It empowers the engineering of complex cellular behaviors, such as spatial patterning based on intercellular communications and processing orthogonal inputs in the same cell. Finally, it was shown that LIDAR is compatible with compact, single-transcript encoding-another unique advantage compared to transcription-based synthetic receptors—and, critically, LIDAR is functional when delivered using mRNA.

### Results

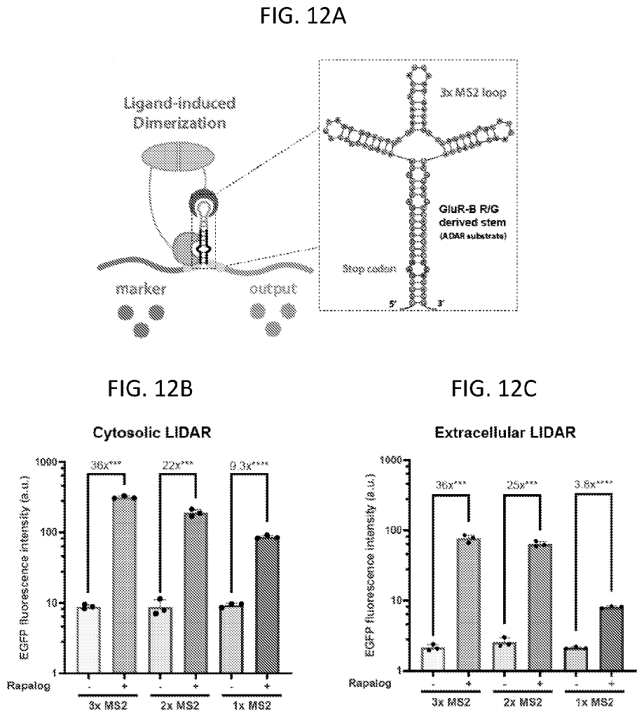

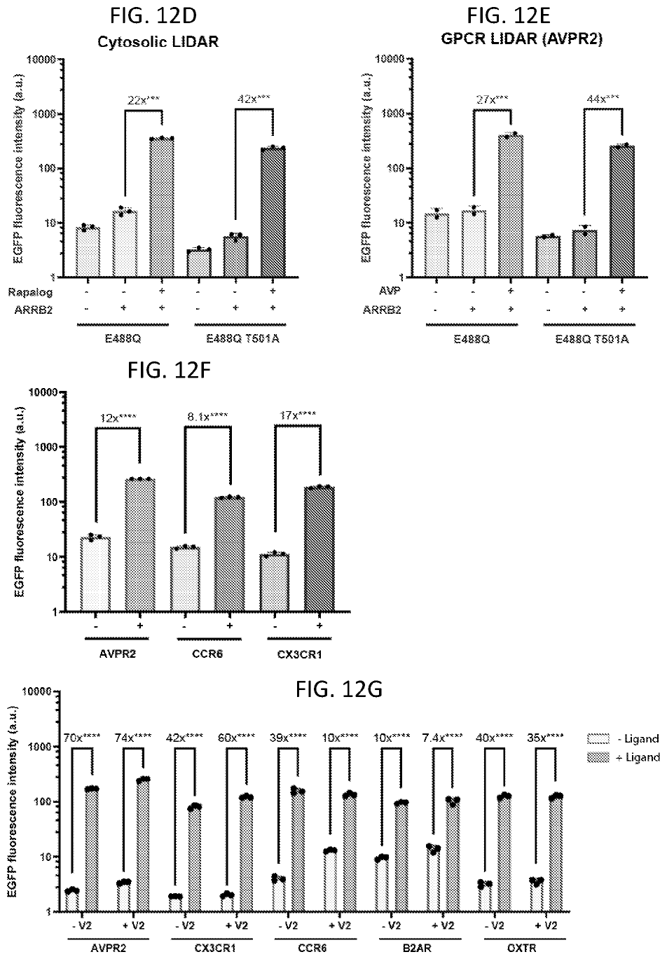

To test the feasibility of LIDAR, FRB and FKBP was started with, Rapalog-responsive dimerization domains (FIG. 8B). To facilitate quantification and iterative engineering, mCherry was encoded upstream of the stop codon as a marker and GFP downstream of the stop codon as the output. The stop codon is located in a short dsRNA sequence modified from the endogenous GluR-B R/G site, a canonical ADAR substrate (6) (FIG. 12A). Several MS2 stem-loops were included to recruit the corresponding phage-derived RBP, MS2 coat protein (MCP).

To maintain a fast design-build-test cycle, unless specified, all prototyping experiments were performed by transiently co-transfecting receptor-encoding DNA plasmids into human embryonic kidney (HEK) 293 cells. Incidentally, it was noticed that LIDAR often exhibits reasonably low baselines even when delivered using transient transfection, in contrast to some other synthetic receptors that tend to show runaway baseline when the components are highly expressed in similar contexts and sometimes require sophisticated gating mechanisms (7).

Optimizations were performed including the addition of self-cleaving F2A peptides to insulate the marker and the output proteins from the non-functional peptide encoded by the ADAR substrate region, testing different numbers of MS2 loops (FIG. 12B-C), and the adoption of double mutations (E488Q. T501A) to improve on-target editing efficiency and reduce nonspecific editing (8, 9) (FIG. 12D-E). A cytosolic LIDAR with minimal leaky baseline and substantial responsiveness to rapalog was achieved (FIG. 8F).

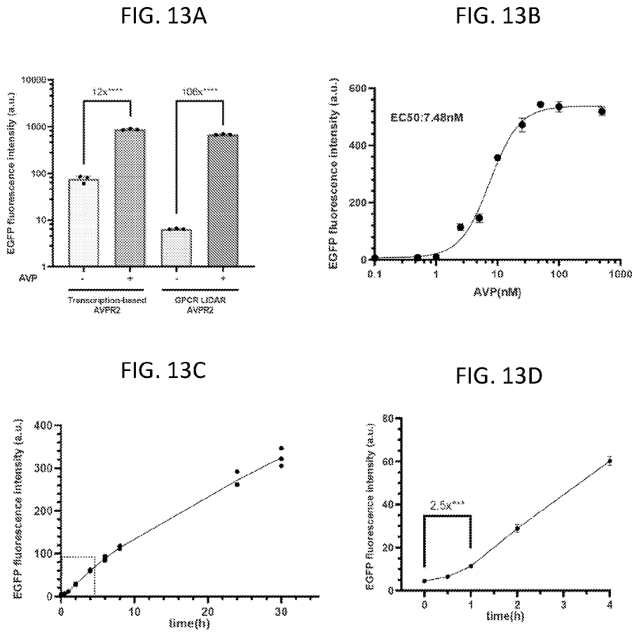

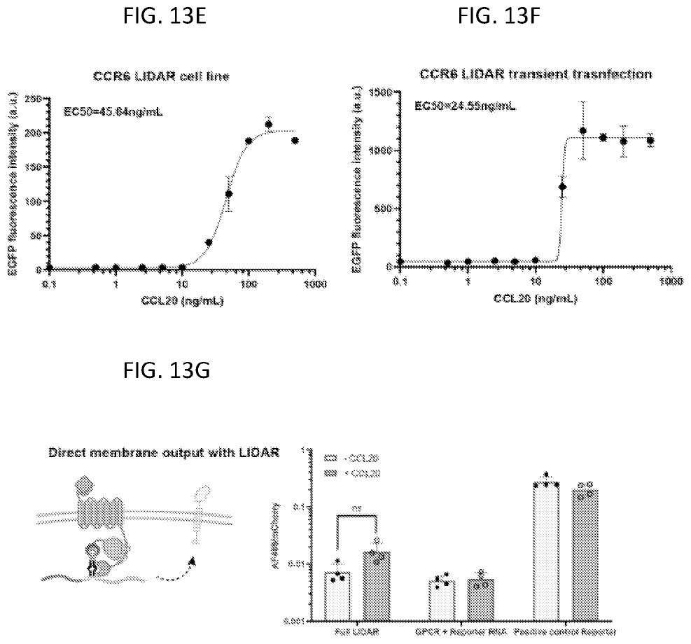

Sensing extracellular ligands was pursued next, while examining the compatibility of LIDAR with a variety of receptor architectures. A CD28 transmembrane domain was added between each of the dimerization domain and actuation domain of the cytosolic LIDAR (FIG. 8C), and validated its response to rapalog (FIG. 8G). Ligand-induced dimerization could also be mediated by protein recruitment upon natural receptor activation. The recruitment of cytosolic β-arrestin2 to the phosphorylated C-terminal tail of a G protein-coupled receptors (GPCR) was harnessed. Using AVPR2, a GPCR for vasopressin (AVP) previously used in TANGO, AVP-responsive LIDAR was validated by fusing ADAR2dd to the C-terminal tail of the GPCR, and MCP to the C-terminus of β-arrestin2 (FIG. 8D, H). The dose response curve (FIG. 13B) is comparable to that of the original TANGO. The “inverted” design that fuses ADAR2dd to β-arrestin2 and MCP to GPCR is also functional, albeit with lower fold activation (FIG. 12F). It was hypothesized that this is because the former design minimizes the background by tethering ADAR2dd to the membrane along with the GPCR, which reduces nonspecific editing on cytosolic reporter RNAs, and promotes the translation of the reporter RNA by tethering it to a cytosolic protein. To generalize LIDAR to other signaling-induced dimerization events, it was demonstrated that LIDAR works with IGFIR, a receptor tyrosine kinase (RTK), and its binding partner Shel (FIG. 8E, I). Therefore, LIDAR can be readily implemented using a variety of architectures and induce robust output upon ligand activation.

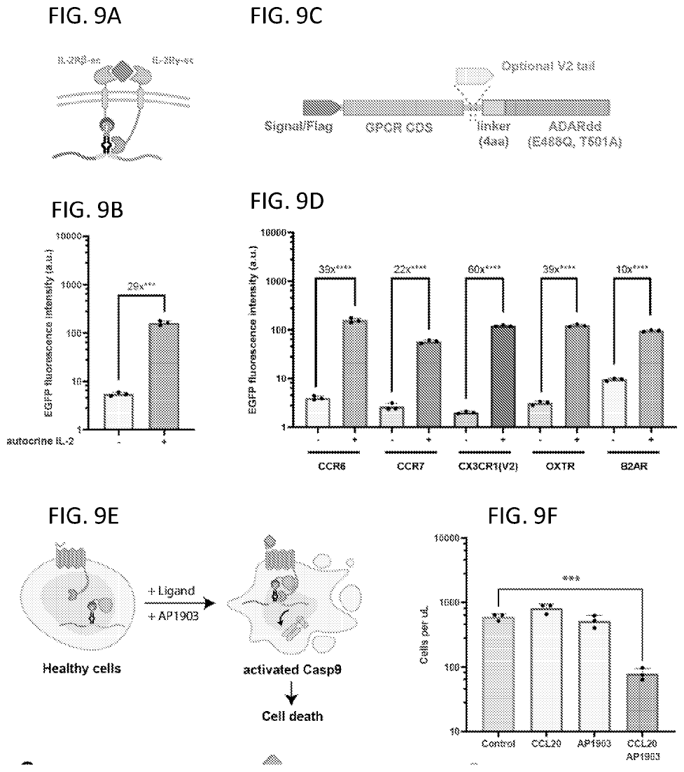

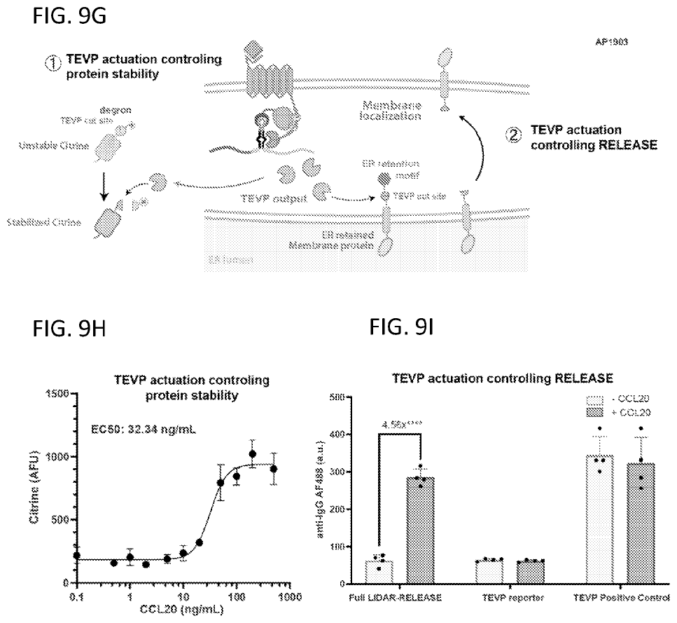

To demonstrate its modularity, it was shown that LIDAR is generalizable within each type of design to sense diverse inputs, as well as to produce functional outputs besides fluorescent proteins. For input versatility, we started with the MESA-like LIDAR and swapped the external dimerization domains to the extracellular domains of IL-2 receptor β and γ and validated its response to autocrine IL-2 (FIG. 9A-B). For TANGO-like LIDAR, a modular design was applied so that the GPCR portion of LIDAR can be easily replaced (FIG. 9C). A wide range of GPCRs are compatible with LIDAR, including chemokine receptors CCR6, CCR7 and CX3CR1, as well as the oxytocin receptor (OXT) and β-2 adrenergic (B2AR) receptor (FIG. 9D and FIG. 12G). It's worth noting that in PRESTO-TANGO (12), an additional V2 tail was included to enhance the signal. For LIDAR, the additional V2 tail increases both the signal and the baseline, and its effect on fold activation is not always beneficial. For GPCRs like AVPR2 and CX3CR that have a low baseline, the V2 tail improves the fold activation by increasing the activation signal; however, for GPCRs like CCR6 and B2AR that exhibit a higher baseline signal, the V2 tail also drastically increases the baseline, reducing the fold activation (FIG. 12G).

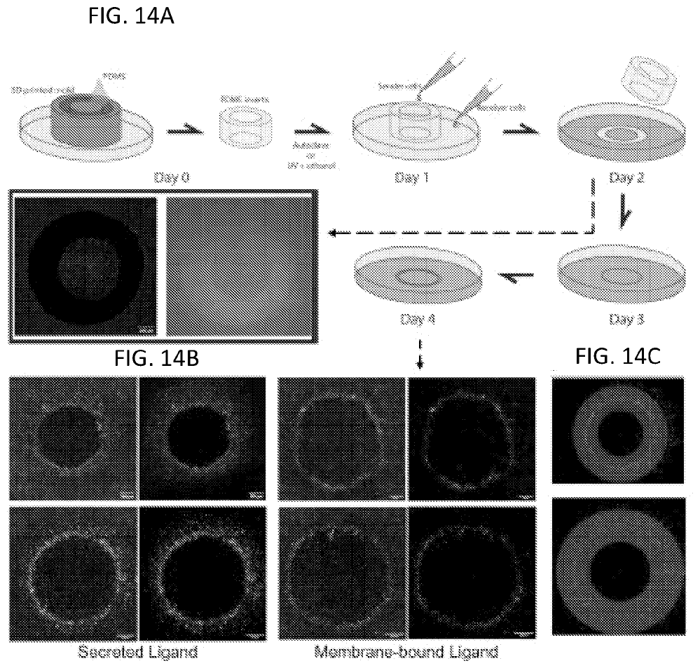

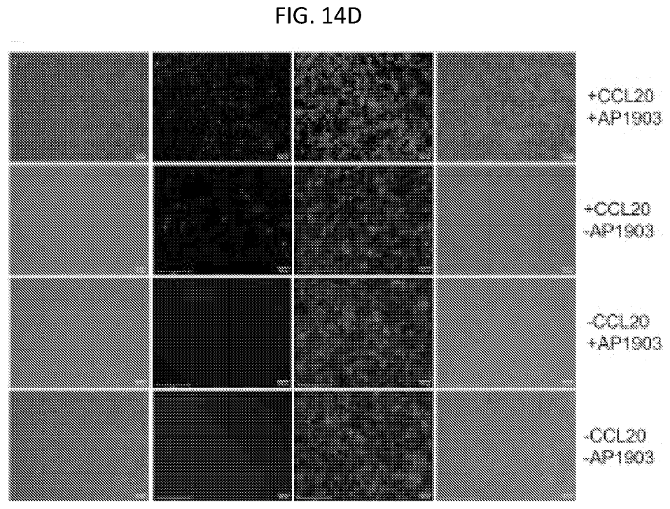

In terms of generalizing functional outputs, LIDAR was used to output iCASP9 as a ligand-dependent cell death controller (13) (FIG. 9E), as iCASP9 induces apoptosis in response to the dimerizing molecule AP1903. A monoclonal cell line was generated (cLSM01-C5) expressing all three components of CCR6-based LIDAR by stable PiggyBac integrations. Substantial cell death was demonstrated only when both the GPCR ligand (CCL20) and AP1903 are present, (FIG. 9F). In parallel with the flow cytometry quantification, verification by cell viability using microscopy reached comparable conclusions (FIG. 14D). Another functional output was then tested, tobacco etch virus (TEV) protease, given our expertise (14) and its broad use (15) in synthetic circuits. A LIDAR-produced TEV protease rescues a fluorescent protein linked via a TEV protease cleavage site to a degron (FIG. 9G). Its dose response curve resembles that of the corresponding LIDAR that directly outputs EGFP (FIG. 9H and FIG. 13F). The results above not only demonstrate that LIDAR could output functional proteases, but the similarity of the dose-responsiveness curves also shows that the terminal output of the system is not dampened by the use of the protease. This indicates the potential for LIDAR to be implemented in more complex circuits (16).

Combined with our RELEASE (Retained Endoplasmic Cleavable Secretion) platform (17), the TEV protease output can be further used to control the surface display of proteins through the removal of endoplasmic reticulum-retention signals (FIG. 9G). Using this strategy, LIDAR's TEV protease output was applied to control the membrane display of a human IgG protein. Baseline IgG display was observed comparable to the negative control where ADAR2dd is not introduced, and ligand-induced IgG display comparable to a positive control that constitutively expresses the TEV protease (FIG. 9I). Besides demonstrating the capability of building complex circuitry using LIDAR. LIDAR-RELEASE also equips LIDAR with the capacity to output membrane or secreted proteins. Taken together, the capability of producing a membrane protein as the output of LIDAR not only demonstrates the composability of the LIDAR system into complex intracellular circuitry, but these transmembrane outputs would also enable intercellular communication and further potentiates building multicellular systems using LIDAR.

Inspired by examples from synNotch (19, 20) and given the advantage that LIDAR could potentially sense both membrane-bound and soluble ligands. LIDAR was used to drive cell-cell interactions to ultimately program complex multicellular behaviors (21-23). CCR6-based LIDAR senses its ligand, CCL20 (FIG. 10A-D), when the receiver cells are either exposed to conditioned media from CCL20-secreting cells (FIG. 10A, B) or co-cultured with cells displaying CCL20 on their surface (FIG. 10C, D). It was tested to determine whether such contact-dependent and soluble ligand sensing features of LIDAR could be used to program distinct spatial patterns as a preliminary demonstration of spatial programming (24). Receiver cells surrounding cells that produce secreted ligands were plated (FIG. 10E), a response gradient was observed going from the sender cells outwards (FIG. 10F). In contrast, when the receiver cells with sender cells producing a membrane-bound ligand were tested, the response pattern is limited to the sender-receiver interface (FIG. 10G, H). The images were quantified to extract the LIDAR output intensity profiles in response to membrane-bound and secreted ligands. As expected, the responses to secreted ligands exhibit longer tails (FIG. 10G) than the response to the membrane ligands, indicating an activation gradient due to the diffusion of the secreted ligand in the former.

To program more complex multi-cellular behaviors, as well as to integrate multiple inputs for targeted cell therapies, it is often required to have receptors that process orthogonal signals in the same cell. In our most commonly used configuration (Tango-like LIDAR), the β-arrestin2-RBP fusion protein is recruited to all GPCRs without discrimination. This means that with two different GPCR-LIDAR receptors (e.g., AVPR-based LIDAR and CX3CR1-based LIDAR) the system would only be able to produce one output, triggered by either ligand. In the inverted version, the ADAR2dd and the RBP are interchanged to obtain the GPCR-RBP and β-arrestin2-ADAR2dd fusion proteins, analogous to TANGO vs. ChaCha receptors (25) (FIG. 10J). Because a separate RBP is needed for each receptor. MCP was used for a reporter RNA harboring MS2 loops and outputting EGFP and PP7 bacteriophage coat protein (PCP) for a reporter RNA harboring PP7 loops and outputting mTagBFP. Using this latter design (ChaCha-like LIDAR), a pair of LIDAR receptors independently triggered in the same cell to produce EGFP exclusively from AVP signaling and mTagBFP exclusively from CX3CL1 signaling (FIG. 10K)

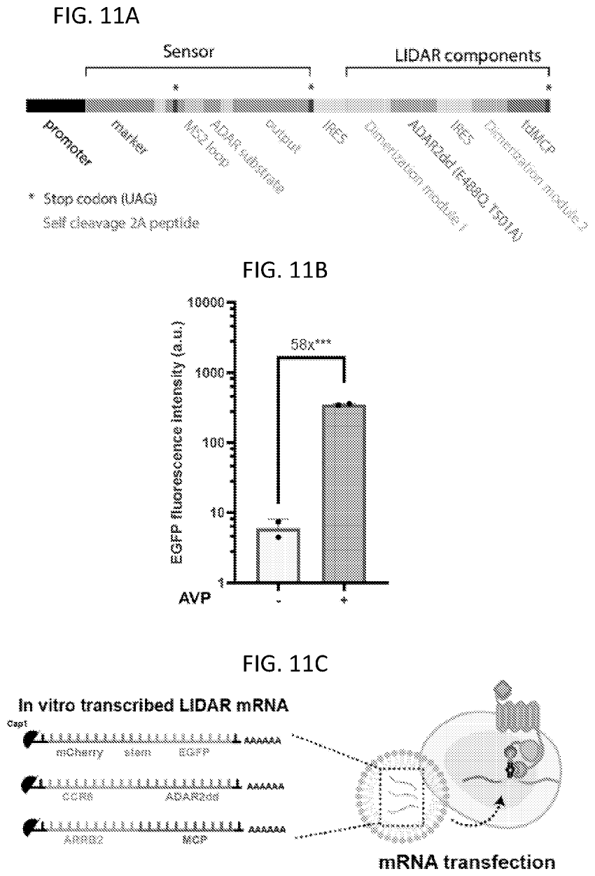

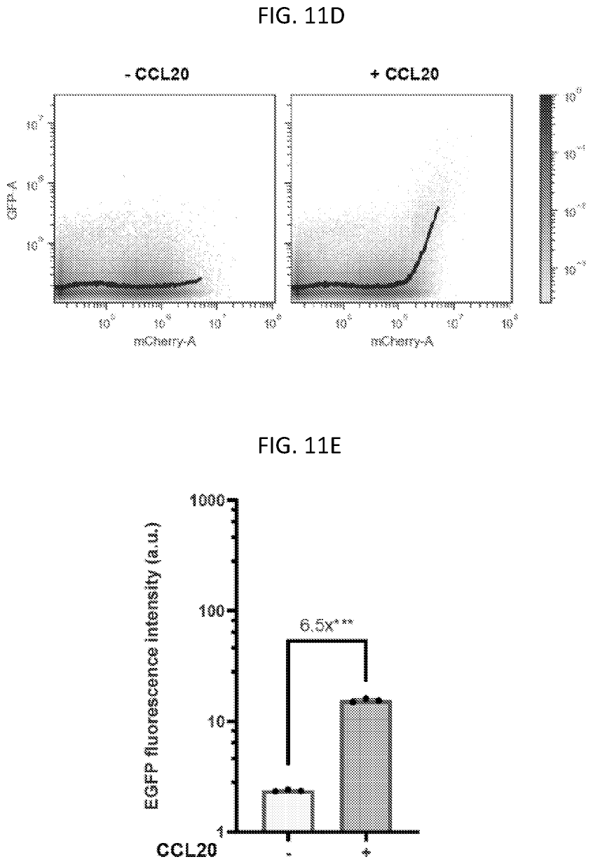

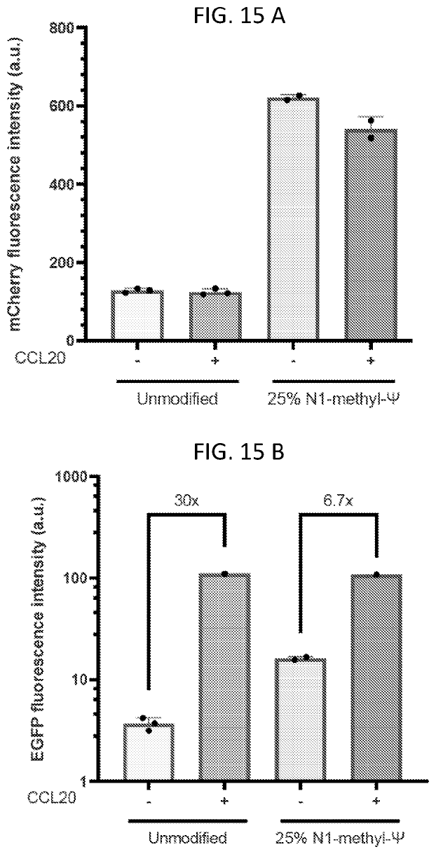

Lastly, it was demonstrated that the unique delivery features enabled by LIDAR's post-transcriptional nature. First, many applications are limited by a delivery bottleneck, a post-transcriptional design could enable the compact encoding of all components, which would increase delivery efficiency by reducing the number of cargos needed and improve performance robustness by “locking in” optimal stoichiometry. All LIDAR components were encoded on a single transcript by separating the components with EMCV IRES sites (FIG. 11A), and indeed observed LIDAR performance comparable to the corresponding multi-transcript version (FIG. 11B, compared to FIG. 8D). Second, given our initial motivation of compatibility with RNA delivery, it was tested whether LIDAR is still functional when encoded and delivered purely by mRNAs transcribed in vitro. It was first tested whether a synthetic reporter mRNA could be edited by ADAR to control output translation. Transfection of the synthetic reporter mRNA into a cell line that stably expresses the two protein components of LIDAR shows that the marker protein is moderately expressed, while the output protein is strongly induced by the ligand (FIG. 15A-B). Incorporation of N1-methyl pseudouridine, a common modification used in mRNA vaccines (26), drastically increases the marker protein expression, but also leads to higher baseline expression of the output EGFP (FIG. 15A-B), presumably due to stop codon skipping induced by uncanonical U on the stop codon (27). Next, the mRNAs were in vitro transcribed to encode the other two LIDAR receptor components (with 100% N1-methyl pseudouridine) and co-transfected all three pieces of LIDAR mRNA into HEK293 cells (FIG. 11C). A significant increase in the EGFP output was observed in response to the ligand (FIG. 11D-E). It is noted that the fold activation is smaller than the DNA-encoded version (FIG. 9D) and that the induction is only observed in the most highly transfected cells (FIG. 11D), suggesting the need for further mRNA-specific optimization. Taken together, the data has shown that LIDAR is indeed the first-in-class post-transcriptional modular synthetic receptor that can be delivered by mRNA, which opens up a brand-new avenue towards RNA-empowered synthetic biology and cell therapy.

This work illustrates that the modular, robust nature of the LIDAR system offers a versatile platform for potential applications in basic research and therapies. Different receptors can be designed using various LIDAR architectures and they can produce functional outputs with good dynamic ranges. Furthermore, it was shown that LIDAR receptors can be incorporated into complex synthetic circuitries and are capable of driving cell-cell interactions to potentially program complex multicellular behavior and functionality. All these desirable features, combined with the validated RNA compatibility, bestows LIDAR with vast potential in the future of in vivo cell therapies positioning it as an attractive tool for the development of smarter cell therapies and biomedical application.

### REFERENCES

1. J. Manhas, H. I. Edelstein, J. N. Leonard, L. Morsut, The evolution of synthetic receptor systems. Nat. Chem. Biol. 18, 244-255 (2022).
2. D. Lee, M. Creed, K. Jung, T. Stefanelli, D. J. Wendler, W. C. Oh, N. L. Mignocchi, C. Lüscher, H.-B. Kwon, Temporally precise labeling and control of neuromodulatory circuits in the mammalian brain. Nat. Methods. 14, 495-503 (2017).
3. J. G. Rurik, I. Tombácz, A. Yadegari, P. O. Méndez Fernández, S. V. Shewale, L. Li, T. Kimura, O. Y. Soliman, T. E. Papp, Y. K. Tam, B. L. Mui, S. M. Albelda, E. Puré, C. H. June, H. Aghajanian, D. Weissman, H. Parhiz, J. A. Epstein, CAR T cells produced in vivo to treat cardiac injury. Science. 375, 91-96 (2022).
4. N. Chaudhary, D. Weissman, K. A. Whitehead, mRNA vaccines for infectious diseases: principles, delivery and clinical translation. Nat. Rev. Drug Discov. 20, 817-838 (2021).
5. K. E. Kaseniit, N. Katz, N. S. Kolber, C. C. Call, D. L. Wengier, W. B. Cody, E. S. Sattely, X. J. Gao, Modular, programmable RNA sensing using ADAR editing in living cells. Nat. Biotechnol. 41, 482-487 (2023).
6. S. Pokharel, P. A. Beal, High-throughput screening for functional adenosine to inosine RNA editing systems. ACS Chem. Biol. 1, 761-765 (2006).
7. J. Zhou, Q. Ge, D. Wang, Q. Guo, Y. Tao, Engineering a modular double-transmembrane synthetic receptor system for customizing cellular programs. Cell Rep. 42, 112385 (2023).
8. D. Katrekar, G. Chen, D. Meluzzi, A. Ganesh, A. Worlikar, Y.-R. Shih, S. Varghese, P. Mali, In vivo RNA editing of point mutations via RNA-guided adenosine deaminases. Nat. Methods. 16, 239-242 (2019).
9. A. S. Thuy-Boun, J. M. Thomas, H. L. Grajo, C. M. Palumbo, S. Park, L. T. Nguyen, A. J. Fisher, P. A. Beal, Asymmetric dimerization of adenosine deaminase acting on RNA facilitates substrate recognition. Nucleic Acids Res. 48, 7958-7972 (2020).
10. N. M. Daringer, R. M. Dudek, K. A. Schwarz, J. N. Leonard, Modular extracellular sensor architecture for engineering mammalian cell-based devices. ACS Synth. Biol. 3, 892-902 (2014).
11. G. Barnea, W. Strapps, G. Herrada, Y. Berman, J. Ong, B. Kloss, R. Axel, K. J. Lee, The genetic design of signaling cascades to record receptor activation. Proc Natl Acad Sci USA. 105, 64-69 (2008).
12. W. K. Kroeze, M. F. Sassano, X.-P. Huang, K. Lansu, J. D. McCorvy, P. M. Giguère, N. Sciaky, B. L. Roth, PRESTO-Tango as an open-source resource for interrogation of the druggable human GPCRome. Nat. Struct. Mol. Biol. 22, 362-369 (2015).
13. Y. Ma, M. W. Budde, M. N. Mayalu, J. Zhu, A. C. Lu, R. M. Murray, M. B. Elowitz, Synthetic mammalian signaling circuits for robust cell population control. Cell. 185, 967-979.e12 (2022).
14. X. J. Gao, L. S. Chong, M. S. Kim, M. B. Elowitz, Programmable protein circuits in living cells. Science. 361, 1252-1258 (2018).
15. H. K. Chung, M. Z. Lin, On the cutting edge: protease-based methods for sensing and controlling cell biology. Nat. Methods. 17, 885-896 (2020).
16. Z. Chen, M. B. Elowitz, Programmable protein circuit design. Cell. 184, 2284-2301 (2021).
17. A. E. Vlahos, J. Kang, C. A. Aldrete, R. Zhu, L. S. Chong. M. B. Elowitz, X. J. Gao, Protease-controlled secretion and display of intercellular signals. Nat. Commun. 13, 912 (2022).
18. C. K. Barlowe, E. A. Miller, Secretory protein biogenesis and traffic in the early secretory pathway. Genetics. 193, 383-410 (2013).
19. L. Morsut, K. T. Roybal, X. Xiong, R. M. Gordley, S. M. Coyle, M. Thomson, W. A. Lim, Engineering customized cell sensing and response behaviors using synthetic notch receptors. Cell. 164, 780-791 (2016).
20. K. T. Roybal, J. Z. Williams, L. Morsut, L. J. Rupp, I. Kolinko, J. H. Choe, W. J. Walker, K. A. McNally, W. A. Lim, Engineering T Cells with Customized Therapeutic Response Programs Using Synthetic Notch Receptors. Cell. 167, 419-432.e16 (2016).
21. Y. Hart, S. Reich-Zeliger, Y. E. Antebi, I. Zaretsky, A. E. Mayo, U. Alon, N. Friedman, Paradoxical signaling by a secreted molecule leads to homeostasis of cell levels. Cell. 158, 1022-1032 (2014).
22. S. Toda, L. R. Blauch, S. K. Y. Tang, L. Morsut, W. A. Lim, Programming self-organizing multicellular structures with synthetic cell-cell signaling. Science. 361, 156-162 (2018).
23. M. Garibyan, T. Hoffman, T. Makasko, S. Do, A. R. March, N. Cho, N. Pedroncolli, R. E. Lima, J. Soto, B. Jackson, A. Khademhosseini, S. Li, M. McCain, L. Morsut, Engineering Programmable Material-To-Cell Pathways Via Synthetic Notch Receptors To Spatially Control Cellular Phenotypes In Multi-Cellular Constructs. BioRxiv (2023), doi: 10.1101/2023.05.19.541497.
24. S. Toda, W. L. McKeithan, T. J. Hakkinen, P. Lopez, O. D. Klein, W. A. Lim, Engineering synthetic morphogen systems that can program multicellular patterning. Science. 370, 327-331 (2020).
25. N. H. Kipniss, P. C. D. P. Dingal, T. R. Abbott, Y. Gao, H. Wang, A. A. Dominguez, L. Labanieh, L. S. Qi, Engineering cell sensing and responses using a GPCR-coupled CRISPR-Cas system. Nat. Commun. 8, 2212 (2017).
26. K. D. Nance, J. L. Meier, Modifications in an Emergency: The Role of N1-Methylpseudouridine in COVID-19 Vaccines. ACS Cent. Sci. 7, 748-756 (2021).
27. J. Karijolich, Y.-T. Yu, Converting nonsense codons into sense codons by targeted pseudouridylation. Nature. 474, 395-398 (2011).

Notwithstanding the appended claims, the disclosure set forth herein is also described by the following clauses:

1. A method of ligand induced ADAR (adenosine deaminase acting on RNA) mediated expression of an RNA coding sequence, the method comprising:
contacting a ligand with a cell associated ligand induced ADAR (LIDAR) mediated expression system comprising:
1) an output RNA comprising:
   (i) a first nucleotide sequence comprising an ADAR responsive site;
   (ii) a second nucleotide sequence encoding a cleavage domain,
   (iii) a third nucleotide sequence encoding an output polypeptide,
2) a stem loop binding protein comprising a stem loop binding domain and a dimerization domain joined by a linker, and
3) an RNA editing protein comprising an RNA editing domain and a dimerization domain joined by a linker;
to express the RNA coding sequence.

2. The method of clause 1, wherein the ligand is an exogenous ligand to the cell.

3. The method of clause 1 wherein the ligand is an endogenous ligand to the cell.

4. The method of any of clauses 1-3, wherein the ADAR responsive site comprises a stem loop sequence comprising one or more stop codons.

5. The method of clause 4, wherein the stem loop further comprises at least 1 base that is mismatched with a sequence within the stem loop opposite the stop codon.

6. The method of any of clauses 1-5, wherein the cell associated LIDAR mediated expression system further comprises a target RNA.

7. The method of clause 6, wherein the ADAR responsive site comprises a sensor nucleotide sequence that is reverse complementary to the target RNA, wherein the sensor nucleotide sequence comprises a stop codon.

8. The method of clause 7, wherein the sensor nucleotide further comprises at least 1 base that is mismatched with the target RNA sequence and a stem loop sequence.

9. The method of clause 7 or 8, wherein the sensor nucleotide sequence is reverse complementary to the 3′ UTR of the target RNA.

10. The method of clauses 7 or 8, wherein the sensor nucleotide sequence is reverse complementary to the coding sequence of the target RNA.

11. The method of any of clauses 7-10, wherein the sensor nucleotide sequence is reverse complementary to two non-contiguous sequences within a single target RNA.

12. The method of any of clauses 7-11, wherein the sensor nucleotide sequence is reverse complementary to two or more distinct target RNAs.

13. The method of any of clauses 1-12, wherein the stem loop sequence comprises two or more MS2 sequence repeats.

14. The method of any of clauses 1-13, wherein the cleavage domain is selected from the group consisting of a 2A self-cleaving domain, a TEV protease cleavage domain, a S1 cleavage domain, a S2 cleavage domain, S3 cleavage domain, and a NS3 cleavage domain.

15. The method of clause 14, wherein the 2A self-cleaving domain is selected from the group consisting of T2A, P2A, E2A and F2A.

16. The method of any of clauses 1-15, wherein the output polypeptide is selected from the group consisting of a fluorescent protein, a luminescent protein, a genomic modification protein, a transcription factor, a killing factor, a toxin, an antigen, a T cell receptor, a cytokine, a chemokine, a growth factor, a signaling peptide, a chimeric antigen receptor and an enzyme.

17. The method of clause 16, wherein the output polypeptide comprises a signal peptide sequence.

18. The method of any of clauses 13-17, wherein the stem loop binding domain binds to the MS2 sequence repeats.

19. The method of any of clauses 1-18, wherein the stem loop binding domain comprises one or more MS2 coat proteins.

20. The method of any of clauses 1-19, wherein the stem loop binding protein and the RNA editing protein are intracellular.

21. The method of any of clauses 1-19, wherein the dimerization domains of the stem loop binding protein and the RNA editing protein are extracellular.

22. The method of clause 21, wherein the stem loop binding domain and the RNA editing domain are intracellular.

23. The method of any of clauses 1-22, wherein the ligand directly binds to the dimerization domains of the stem loop binding protein and the RNA editing protein

24. The method of any of clauses 1-19, wherein the stem loop binding protein is intracellular.

25. The method of clause 24, wherein dimerization domain of the RNA editing protein is extracellular and the RNA editing domain is intracellular.

26. The method of clauses 24 or 25, wherein the dimerization domain is post-translationally modified after binding to the ligand.

27. The method of any of clauses 24-26, wherein the dimerization domain of the RNA editing protein further comprises a V2 tail.

28. The method of any of clauses 24-26, wherein the dimerization domain of the stem loop binding protein further comprises a V2 tail.

29. The method of any of clauses 1-28, wherein the RNA editing domain comprises an adenosine deaminase acting on RNA (ADAR) protein.

30. The method of clause 29, wherein the ADAR protein is the deaminase domain of ADAR1 or ADAR2.

31. The method of clause 30, wherein the deaminase domain is a modified deaminase domain that has increased editing activity.

32. The method of clause 31, wherein the modified deaminase domain is a T501A deaminase domain variant.

33. The method of any of clauses 1-32, wherein the dimerization domain of the stem loop binding protein is selected from the group consisting of a natural binding domain, an engineered binding domain, FRB, FKBP, IL-2Rb-ec, IL-2Rg-ec, IL-10Ra-ec, IL-10Rb-ec, a G-protein coupled receptor (GPCR), a receptor tyrosine kinase (RTK) or a fragment thereof, a chemokine receptor, an opioid receptor, an insulin-like growth factor receptor, b-arrestin. Shc, a rhodopsin, a visual arrestin, a receptor tyrosine kinase or fragment thereof, an antibody or fragment thereof that specifically bind to the ligand and an antibody or a fragment thereof that specifically binds to the dimerization domain of the RNA editing protein.

34. The method of clause 33, wherein the antibody or a fragment thereof only binds to the RNA editing protein when the dimerization domain of the RNA editing protein is bound to the ligand.

35. The method of any of clauses 1-34, wherein the dimerization domain of the RNA editing protein is selected from the group consisting of FRB, FKBP, IL-2Rb-ec, IL-2Rg-ec, IL-10Ra-ec, IL-10Rb-ec, a GPCR, a RTK, a chemokine receptor, an opioid receptor, an insulin-like growth factor receptor, b-arrestin, She, a rhodopsin, a visual arrestin, a receptor tyrosine kinase or fragment thereof, and or a fragment thereof that specifically binds to the dimerization domain of the stem loop binding protein.

36. The method of clause 35, wherein the antibody or a fragment thereof only binds to the stem loop protein when the dimerization domain of the RNA editing protein is bound to the ligand.

37. The method of any of the preceding clauses, wherein the output RNA is a circularized RNA.

38. The method of any of clauses 1-37, wherein the output RNA is contained within a delivery vehicle.

39. The method of clause 37, wherein the delivery vehicle is a lipid nanoparticle.

40. The method of clause 39, wherein the lipid nanoparticle further comprises an RNA encoding the stem loop binding protein.

41. The method of clauses 39 or 40, wherein the lipid nanoparticle further comprises an RNA encoding the RNA editing protein.

42. The method of any of clauses 1-41, wherein the contacting comprises transfecting the cell with a recombinant vector comprising the output RNA, an RNA encoding the stem loop binding protein, an RNA encoding the RNA editing protein, or a combination thereof.

43. The method of any of clauses 1-42, wherein the linker of the RNA editing protein is 6 amino acids or less.

44. The method of any of clauses 1-43, wherein the linker of the stem loop binding protein is 6 amino acids or less.

45. The method of any of clauses 1-44, wherein the output RNA further comprises a fourth nucleotide sequence encoding a marker protein wherein the fourth nucleotide sequence precedes the first nucleotide sequence.

46. The method of clause 45, wherein the marker protein is a fluorescent protein or a luminescent protein.

47. The method of any of clauses 1-46, wherein the output RNA further comprising a fifth nucleotide sequence encoding a second cleavage domain wherein the fifth nucleotide sequence precedes the first nucleotide sequence and is after the fourth nucleotide sequence.

48. The method of clause 47, wherein the cleavage domain is a 2A self-cleaving domain.

49. The method of clause 48, wherein the 2A self-cleaving domains is selected from the group of T2A, P2A, E2A and F2A.

50. The method of any of the preceding clauses, wherein the expression of the RNA coding sequences treats a disease or a condition.

51. The stem loop binding protein of any of the preceding clauses.

52. The RNA editing protein of any of the preceding clauses.

53. A recombinant vector comprising the output RNA of any of the preceding clauses.

54. A recombinant vector comprising a nucleic acid sequence encoding the stem loop binding protein of any of the preceding clauses.

55. A recombinant vector comprising a nucleic acid sequence encoding the RNA editing protein of any of the preceding clauses.

56. A recombinant vector comprising the output RNA, a nucleic acid sequence encoding the stem loop binding protein and a nucleic acid sequence encoding the RNA editing protein according to any of the preceding clauses.

57. The recombinant vector of any of clauses 53-55, wherein the recombinant vector is selected from the group of a plasmid, a viral vector, a cosmid and an artificial chromosome.

58. A composition comprising the cell associated LIDAR mediated expression system of any of the preceding clauses.

59. The composition of clause 58, wherein the composition is contained with a delivery vehicle.

60. The composition of clause 59, wherein the delivery vehicle is a lipid nanoparticle.

61. A kit, the kit comprising the composition of any of clauses 56-58.

62. The kit of clause 61, further comprising a ligand that specifically binds to the dimerization domain of the RNA editing protein.

63. The kit of clause 61, further comprising a ligand that specifically binds to the dimerization domain of the stem loop binding protein.

64. A kit, the kit comprising:
a stem loop binding protein comprising a stem loop binding domain and a dimerization domain joined by a linker or a nucleic acid encoding the same, and
an RNA editing protein comprising an RNA editing domain and a dimerization domain joined by a linker or a nucleic acid encoding the same.

65. The kit of clause 64, wherein the stem loop binding domain binds to the MS2 sequence repeats.

66. The kit of clauses 64 or 65, wherein the stem loop binding domain comprises one or more MS2 coat proteins.

67. The kit of any of clauses 64-66, wherein the stem loop binding protein and the RNA editing protein are intracellular.

68. The kit of any of clauses 64-67, wherein the dimerization domains of the stem loop binding protein and the RNA editing protein are extracellular.

69. The kit of clause 68, wherein the stem loop binding domain and the RNA editing domain are intracellular.

70. The kit of any of clauses 64-69, wherein the ligand directly binds to the dimerization domains of the stem loop binding protein and the RNA editing protein
71. The kit of any of clauses 64-66, wherein the stem loop binding protein is intracellular.

72. The kit of clause 71, wherein dimerization domain of the RNA editing protein is extracellular and the RNA editing domain is intracellular.

73. The kit of clauses 71 or 72, wherein the dimerization domain is post-translationally modified after binding to the ligand.

74. The kit of any of clauses 71-73, wherein the RNA editing protein further comprises a V2 tail.

75. The kit of any of clauses 64-74, wherein the RNA editing domain comprises an adenosine deaminase acting on RNA (ADAR) protein.

76. The kit of clause 75, wherein the ADAR protein is the deaminase domain of ADAR1 or ADAR2.

77. The kit of clause 75, wherein the deaminase domain is a modified deaminase domain that has increased editing activity.

78. The kit of clause 77, wherein the modified deaminase domain is a T501A deaminase domain variant.

79. The kit of any of clauses 64-78, wherein the dimerization domain of the stem loop binding protein is selected from the group consisting of a natural binding domain, an engineered binding domain, FRB, FKBP, IL-2Rb-ec, IL-2Rg-ec, IL-10Ra-ec, IL-10Rb-ec, a G-protein coupled receptor (GPCR), a receptor tyrosine kinase (RTK) or a fragment thereof, a chemokine receptor, an opioid receptor, an insulin-like growth factor receptor, b-arrestin. Shc, a rhodopsin, a visual arrestin, a receptor tyrosine kinase or fragment thereof, an antibody or fragment thereof that specifically bind to the ligand and an antibody or a fragment thereof that specifically binds to the dimerization domain of the RNA editing protein.

80. The kit of clause 79, wherein the antibody or a fragment thereof only binds to the RNA editing protein when the dimerization domain of the RNA editing protein is bound to the ligand.

81. The kit of any of clauses 64-80, wherein the dimerization domain of the RNA editing protein is selected from the group consisting of FRB, FKBP, IL-2Rb-ec, IL-2Rg-ec, IL-10Ra-ec, IL-10Rb-ec, a GPCR, a RTK, a chemokine receptor, an opioid receptor, an insulin-like growth factor receptor, b-arrestin, Shc, a rhodopsin, a visual arrestin, a receptor tyrosine kinase or fragment thereof, and or a fragment thereof that specifically binds to the dimerization domain of the stem loop binding protein.

82. The kit of clause 81, wherein the antibody or a fragment thereof only binds to the stem loop protein when the dimerization domain of the RNA editing protein is bound to the ligand.

83. The kit of any of clauses 64-82, wherein the kit comprises the nucleic acid sequence encoding the stem loop binding protein and the nucleic acid sequence encoding the RNA editing protein.

84. The kit of any of clauses 64-82, wherein the kit comprises the stem loop protein and the RNA editing protein.

85. The kit of any of clauses 64-84, further comprising a ligand that specifically binds to the dimerization domain of the dimerization domain of the RNA editing protein.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

1. A method of ligand induced ADAR (adenosine deaminase acting on RNA) mediated expression of an RNA coding sequence, the method comprising:
contacting a ligand with a cell associated ligand induced ADAR (LIDAR) mediated expression system comprising:
1, an output RNA comprising:
(i) a first nucleotide sequence comprising an ADAR responsive site;
(ii) a second nucleotide sequence encoding a cleavage domain, and
(iii) a third nucleotide sequence encoding an output polypeptide,
2, a stem loop binding protein comprising a stem loop binding domain and a dimerization domain joined by a linker, and
3, an RNA editing protein comprising an RNA editing domain and a dimerization domain joined by a linker;
to express the RNA coding sequence.

2. The method of claim 1, wherein the ligand is an exogenous ligand to the cell.

3. The method of claim 1, wherein the ligand is an endogenous ligand to the cell.

4. The method of claim 1, wherein the ADAR responsive site comprises a stem loop sequence comprising one or more stop codons.

5. The method of claim 4, wherein the stem loop further comprises at least 1 base that is mismatched with a sequence within the stem loop opposite the stop codon.

6. The method of claim 1, wherein the stem loop sequence comprises two or more MS2 sequence repeats.

7. The method of claim 1, wherein the cleavage domain is a 2A self-cleaving domain selected from the group consisting of T2A, P2A, E2A and F2A.

8. The method of claim 1, wherein the output polypeptide is selected from the group consisting of a fluorescent protein, a luminescent protein, a genomic modification protein, a transcription factor, a killing factor, a toxin, an antigen, a T cell receptor, a cytokine, a chemokine, a growth factor, a signaling peptide, a chimeric antigen receptor, a protease, and an enzyme.

9. The method of claim 1, wherein the stem loop binding domain comprises one or more MS2 coat proteins.

10. The method of claim 1, wherein the stem loop binding protein and the RNA editing protein are intracellular.

11. The method of claim 1, wherein the dimerization domains of the stem loop binding protein and the RNA editing protein are extracellular.

12. The method of claim 11, wherein the stem loop binding domain and the RNA editing domain are intracellular.

13. The method of claim 1, wherein the ligand directly binds to the dimerization domains of the stem loop binding protein and the RNA editing protein

14. The method of claim 1, wherein the stem loop binding protein is intracellular.

15. The method of claim 14, wherein dimerization domain of the RNA editing protein is extracellular and the RNA editing domain is intracellular.

16. The method of claim 14, wherein the dimerization domain is post-translationally modified after binding to the ligand.

17. The method of claim 1, wherein the RNA editing domain comprises a deaminase domain of ADAR1 or ADAR2.

18. The method of claim 17, wherein the deaminase domain is a T501A E488Q deaminase domain variant.

19. The method of claim 1, wherein the dimerization domain of the stem loop binding protein is selected from the group consisting of a natural binding domain, an engineered binding domain, FRB, FKBP, IL-2Rb-ec, IL-2Rg-ec, IL-10Ra-ec, IL-10Rb-ec, a G-protein coupled receptor (GPCR), a receptor tyrosine kinase (RTK) or a fragment thereof, a chemokine receptor, an opioid receptor, an insulin-like growth factor receptor, b-arrestin, Shc, a rhodopsin, a visual arrestin, a receptor tyrosine kinase or fragment thereof, an antibody or fragment thereof that specifically bind to the ligand and an antibody or a fragment thereof that specifically binds to the dimerization domain of the RNA editing protein.

20. The method of claim 1, wherein the dimerization domain of the RNA editing protein is selected from the group consisting of FRB, FKBP, IL-2Rb-ec, IL-2Rg-ec, IL-10Ra-ec, IL-10Rb-ec, a GPCR, a RTK, a chemokine receptor, an opioid receptor, an insulin-like growth factor receptor, b-arrestin, Shc, a rhodopsin, a visual arrestin, a receptor tyrosine kinase or fragment thereof, and or a fragment thereof that specifically binds to the dimerization domain of the stem loop binding protein.
